(19) United States
(12) Patent Application Publication
(10) Pub. No.: US 2004/0235162 A1
(43) Pub. Date: Nov. 25, 2004

(54) Method of preparing immunoregulatory dendritic cells and the use thereof

(75) Inventor: Katsuaki Sato, Kagoshima (JP)

Correspondence Address: FOLEY AND LARDNER, SUITE 500, 3000 K STREET NW, WASHINGTON, DC, 20007, US

(73) Assignees: KIRIN BEER KABUSHIKI KAISHA; Katsuaki SATO

(21) Appl. No.: 10/740,834

(22) Filed: Dec. 22, 2003

(30) Foreign Application Priority Data
Mar. 18, 2003 (JP) 073799/2003

(51) Int. Cl. C12N 5/08

(52) U.S. Cl. 435/372

(57) ABSTRACT

This invention provides: a therapeutic agent for graft rejection, graft-versus-host disease, autoimmune disease, allergic disease, or other diseases comprising dendritic cells (DCs) induced under culture conditions comprising both IL-10 and TGF-β or DCs prepared by adding inflammatory stimulation (e.g., TNF-α or LPS) to the aforementioned DCs and, if necessary, an antigen associated with a target disease; a method of inducing human immunoregulatory dendritic cells by culturing human dendritic cells or their precursor cells in vitro with cytokines comprising at least IL-10 and TGF-β; human immunoregulatory dendritic cells obtained by such method; and a pharmaceutical composition comprising such human immunoregulatory dendritic cells.

### TECHNICAL FIELD

The present invention relates to a therapeutic agent for graft rejection, graft-versus-host disease, autoimmune disease, allergic disease, or other diseases comprising dendritic cells (DCs) induced under culture conditions comprising at least both of IL-10 and TGF-β or DCs prepared by adding inflammatory stimulation (e.g., TNF-α or LPS) to the aforementioned DCs and, if necessary, an antigen associated with a target disease.

### BACKGROUND ART

Dendritic cells (DCs) are the most potent antigen-presenting cells in an organism, and they are known to induce immune responses by presenting an antigen to T cells. DCs are known to act directly not only on T cells but also on B cells, NK cells, NKT cells, and other cells, and they play major roles in immune reactions (Hart, D. N. J., Blood 1997, 90: 3245-3278). Immature DCs exist in peripheral tissues, and they are highly capable of incorporating antigens, although their capability of stimulating T cells is low. When immature DCs receive infectious or inflammatory stimuli, they cause costimulating molecules such as CD40, CD80, or CD86 to be expressed with higher frequency, and they acquire high capability of stimulating T cells. At the same time, they are transferred to peripheral lymphatic tissues, and they activate T cells specific to the antigens incorporated, thereby inducing immune responses (Banchereau, J. et al., Annu. Rev. Immunol. 2000, 18: 767-811). A technique of inducing DCs in vitro is being established, and cancer-specific antigens have been successively identified. Based on these achievements, research aimed at the application of the potent capability of DCs in terms of inducing immunity to the treatment of cancer is expanding. Such novel cellular medicine has drawn attention as future medicine, and development thereof is expected.

In a healthy organism, mechanisms of immune response function against foreign (“nonself”) antigens or tumors to eliminate them. However, immune tolerance to normal self-antigens constituting an organism or harmless foreign antigens can be established, and eliminative mechanisms of immune responses may not function against them. Regulatory mechanisms against autoimmune, allergic, or other diseases, which are disturbed for any reason, are considered to cause such diseases. DCs play major roles not only as antigen presenting cells in the establishment of immune responses against infections or cancer, but they also play important roles in induction of immune tolerance (Steinman, R. M., Proc. Natl. Acad. Sci. USA 2002, 99: 351-358). Immune tolerance is roughly classified into two types: elimination of self-reactive T cell clones in thymic glands, which is referred to as central tolerance; and regulation of self-reactive T cells outside the thymic glands, which is a mechanism referred to as peripheral tolerance. In particular, the latter is known to induce cell death or anergy to self-antigens and to have active suppressing mechanisms mediated by immunoregulatory T cells (Roncarolo, M. G. and Levings M. K., 2000, Curr. Opinion Immunol. 12: 676-683). DCs have been found to be capable of inducing cell death and anergy to T cells and to be capable of inducing immunoregulatory T cells. Clarification of the way that DCs acquire such functional multidimensionality for immune response regulation is making progress in terms of, for example, differences in the maturation phase of DCs, existence of a subset of DCs with different functions, and types of stimuli such as cytokines or pathogens. Based on these findings, application of the capability of DCs to induce immune tolerance to the treatment of autoimmune disease or suppression of graft rejection has been examined (Jonuleit, H. et al., Trends in Immunol. 2001, 22: 394-400; Hackstein, H. et al., Trends in Immunol. 2001, 22: 437-442).

Immature DCs induced from human monocytes with the aid of GM-CSF and IL-4 were further cultured in GM-CSF and IL-10 for 2 days. In the thus prepared DCs, expression levels of costimulating molecules CD56, CD86, and CD83 are lower, and growth of CD4⁺ T cells caused by an allogeneic mixed leukocyte reaction (allo-MLR) is suppressed (Steinbrink, K. et. al., J. Immunol. 1997, 159: 4722-4780). Immature DCs induced from human monocytes with the aid of GM-CSF and IL-4 were further cultured in GM-CSF and IL-10 for 2 days, and the thus prepared DCs induce CD4⁺ and CD8⁺ T cells that are anergic against antigenic stimuli and have immune-suppressing activity (Steinbrink, K. et. al., Blood 2002, 99: 2468-2476). When naive CD4⁺ T cells are stimulated repeatedly with immature human DCs, CD4⁺ CD25⁺ immunoregulatory T cells highly capable of producing IL-10 are induced. These T cells suppress antigen-specific growth of activated Th1 cells (type I helper T cells) and production of cytokines in vitro (Jonuleit, H. et al., J. Exp. Med. 2000, 192: 1213-1222). Administration of immature human DCs comprising a peptide derived from the influenza virus matrix protein to a healthy individual resulted in suppression of the aforementioned antigen-specific CD8⁺ T cells and in induction of the aforementioned antigen-specific CD8⁺ IL-10-producing immunoregulatory T cells. This suppressing effect, however, disappeared 6 months after administration of DCs (Dhodapkar, M. V. et al., J. Exp. Med. 2001, 193: 233-238; Dhodapkar, M. V. et al., Blood 2002, 100: 174-177). Thus, it is suggested that immature DCs induced from human monocytes with the aid of GM-CSF and IL-4 or DCs maintain their immature states by being treated with IL-10 induce antigen-specific immune suppression. Under inflammatory conditions such as those of autoimmune diseases, however, immature DCs are induced to mature, and whether or not DCs are capable of maintaining immunoregulatory functions is an issue in question.

Murine bone marrow-derived DCs induced with the aid of GM-CSF and TGF-β1 have features of immature DCs and have attenuated activity for accelerating growth of allogeneic and naive CD4⁺ T cells (Yamaguchi, Y. et al., Stem Cells. 1997, 15(2): 144-53). The aforementioned DCs were administered to a mouse allogeneic heart transplant model to prolong graft survival (Lu, L. et. al., Transplantation 1997, 64: 1808-1815). Similarly, GM-CSF-induced murine bone marrow-derived immature DCs and GM-CSF-induced murine liver-derived immature DCs exhibited effects of prolonging graft survival in an allogeneic transplant model (Lutz, M. et al., Eur. J. Immunol. 2000, 30: 1813-1822; Fu, F. et al., Transplantation 1996, 62: 659-665; Rastellini, C. et al. Transplantation 1995, 60: 1366-1370). In contrast, murine spleen-derived CD8⁺ DCs exhibited effects of prolonging graft survival in an allogeneic transplant model regardless of its maturation phase (O'Connell, P. J. et al. J. Immunol. 2002, 168: 143-154). The effects of suppressing autoimmune diabetes in a NOD mouse were observed only when mature DCs were administered instead of immature DCs (Feili-Hariri, M. et al., Eur. J. Immunol. 2002, 32: 2021-2030). In the case of EAE multiple sclerosis models, effects of suppressing the target disease by semi-mature DCs treated with TNF-α for a short period of time have been reported (Menges, M. et al., J. Exp. Med. 2002, 195: 15-21). Thus, it is difficult to judge the immune-suppressing properties of DCs depending on their maturation phases in research utilizing murine DCs. Concerning murine DCs, effects of suppressing a target disease in a transplant model to which a gene of a molecule associated with suppression and regulation of immune responses, such as FasL, CTLA-4-Ig, IL-10, TGF-β, and IL-4, have been introduced and in an autoimmune disease model have been reported (Hackstein, H. et al., Trends in Immunol. 2001, 22: 437-442). DCs induced from murine bone marrow cells with the aid of GM-CSF and IL-4 were used, IL-10-transduced DCs and TGF-β-transduced DCs were mixed with each other, and the resultant was administered in the portal vein. The effects thereof for prolonging graft survival were observed in an allogeneic kidney transplant model (Gorczynski, R. M. et al., Clin. Immunol. 2000, 95: 182-189).

List of References

Jonuleit, H. et al., Trends in Immunol. 2001, 22: 394-400

Hackstein, H. et al., Trends in Immunol. 2001, 22: 437-442

Steinbrink, K. et al., J. Immunol. 1997, 159: 4722-4780

Steinbrink, K. et al., Blood 2002, 99: 2468-2476

Jonuleit, H. et al., J. Exp. Med. 2000, 192: 1213-1222

Dhodapkar, M. V. et al., J. Exp. Med. 2001, 193: 233-238

Dhodapkar, M. V. et al., Blood 2002, 100: 174-177

Yamaguchi, Y. et al., Stem Cells. 1997, 15 (2): 144-53

Lutz, M. et al., Eur. J. Immunol. 2000, 30: 1813-1822

Fu, F. et al., Transplantation 1996, 62: 659-665

Rastellini, C. et al. Transplantation 1995, 60: 1366-1370

O'Connell, P. J. et al. J. Immunol. 2002, 168: 143-154

Feili-Hariri, M. et al., Eur. J. Immunol. 2002, 32: 2021-2030

Menges, M. et al., J. Exp. Med. 2002, 195: 15-21

Hackstein, H. et al., Trends in Immunol. 2001, 22: 437-442

Gorczynski, R. M. et al., Clin. Immunol. 2000, 95: 182-189

### DISCLOSURE OF THE INVENTION

Objects of the present invention are to provide human immunoregulatory dendritic cells having immunoregulatory properties even under inflammatory disease conditions, a method of inducing the immunoregulatory dendritic cells, and a pharmaceutical composition comprising the immunoregulatory dendritic cells. More particularly, objects of the present invention are to provide a method of culturing human dendritic cells or their precursor cells in the presence of cytokines comprising at least IL-10 and TGF-β, thereby inducing human immunoregulatory dendritic cells, human immunoregulatory dendritic cells induced by the aforementioned method, and the use of the aforementioned immunoregulatory dendritic cells for treating graft rejection, graft-versus-host disease, autoimmune disease, and allergic disease.

As mentioned above, attempts have been heretofore made to obtain dendritic cells (DCs) having immune-suppressing activity. These dendritic cells, however, merely maintained their immature states, and maturation thereof was disadvantageously induced under inflammatory conditions. Thus, whether or not they could maintain immune-suppressing activity was an issue of concern. The present inventor has conducted concentrated studies in order to induce dendritic cells that can sufficiently function even under inflammatory disease conditions. As a result, he has found that immunoregulatory DCs induced with the aid of IL-10 in combination with TGF-β have effects of inducing immune responses and potently suppressing the target disease in a disease model. Further, the present inventor has examined the usefulness of the aforementioned immunoregulatory DCs in terms of treatment of graft rejection or diseases associated with immunity, and as a result, he has found that the DCs have effects of suppressing graft rejection and treating immune-associated diseases. This has led to the completion of the present invention.

More specifically, the present invention is as follows:

[1] a method of preparing human immunoregulatory dendritic cells by culturing human dendritic cells or their precursor cells in vitro with IL-10 and TGF-β;

[2] the method of preparing human immunoregulatory dendritic cells according to [1], wherein the human dendritic cells are derived from human monocytes;

[3] a method of preparing human immunoregulatory dendritic cells comprising culturing human monocytes in the presence of GM-CSF, IL-4, IL-10, and TGF-β;

[4] the method of preparing human immunoregulatory dendritic cells according to [3], wherein culture is further conducted in the presence of at least one of TNF-α and LPS;

[5] the method of preparing human immunoregulatory dendritic cells according to any of [1] to [4], wherein culture is further conducted in the presence of an antigen existing in a tissue or organ associated with a disease to be treated;

[6] the method of preparing human immunoregulatory dendritic cells according to [5], wherein the disease is an autoimmune or allergic disease;

[7] human immunoregulatory dendritic cells prepared by the method according to any of [1] to [6];

[8] the human immunoregulatory dendritic cells according to [7], wherein expression levels of CD83, CD40, CD80, and CD86 are significantly lower than those in mature human dendritic cells that were not cultured in the presence of both IL-10 and TGF-β;

[9] the human immunoregulatory dendritic cells according to [7] or [8], which are capable of inducing antigen-specific anergy to allogeneic CD4⁺ T cells in vitro, suppressing reactivation of activated allogeneic CD4⁺ T cells and CD8⁺ T cells, inducing allogeneic and naive CD4⁺ T cells and CD8⁺ T cells to become CD4⁺ CD25⁺ immunoregulatory T cells and CD8⁺ CD28⁻ immunoregulatory T cells, respectively, and inducing immune-suppressing responses such as the suppression of graft-versus-host disease after xenogeneic transplantation in a human T cell-transplanted immunodeficient mouse through administration of the aforementioned cells to which xenoantigens have been imparted;

[10] a pharmaceutical composition comprising the human immunoregulatory dendritic cells according to any of [7] to [9];

[11] the pharmaceutical composition according to [10], which suppresses graft rejection caused along with cell, organ, or tissue transplantation;

[12] the pharmaceutical composition according to [10], which can be used for treating graft-versus-host disease; and

[13] the pharmaceutical composition according to [10], which can be used for treating an autoimmune or allergic disease.

The present invention is hereafter described in detail.

1. Preparation of Human Immunoregulatory DCs

The present invention relates to a method of preparing human immunoregulatory DCs by culturing human dendritic cells (DCs) or their precursor cells in the presence of cytokines comprising at least IL-10 and TGF-β and to human immunoregulatory DCs obtained by the aforementioned method. For example, GM-CSF, IL-4, IL-10, or TGF-β1 is added to human monocytes to induce DCs, and inflammatory stimuli (such as TNF-α or LPS) are further added to the thus prepared DCs to induce another type of DCs. The thus obtained DCs have human immunoregulatory properties. Human monocytes are cultured in vitro in the presence of GM-CSF and IL-4, human monocytes are then differentiated to result in DCs, and DCs become immature immunoregulatory DCs with the aid of IL-10 and TGF-β. In such a case, human monocytes may be first stimulated with GM-CSF and IL-4 for differentiation to DCs, followed by stimulation with IL-10 and TGF-β. Alternatively, human monocytes may be simultaneously stimulated with GM-CSF, IL-4, IL-10, and TGF-β1. Further, immature human immunoregulatory DCs become mature human immunoregulatory cells through application of inflammatory stimuli such as TNF-α or LPS.

Human DCs can be obtained by culturing human monocytes in the presence of GM-CSF and IL-4 as described above. In this case, monocytes may be derived from human peripheral blood, human bone marrow, human spleen cells, or human umbilical cord blood. Further, dendritic cells can be isolated from these tissues or organs using a fluorescent activated cell sorter (FACS) or a flowcytometer while employing expression of DC-specific surface antigen, such as CD1a, as an indicator. A specific cell group can be isolated using FACS in accordance with a known technique. Examples of FACS and a flowcytometer that can be used are the FACSVantage (Becton Dickinson) and FACSCalibur (Becton Dickinson).

Human monocytes and DCs can be cultured in accordance with a known culture technique for human lymphoid cells. For example, RPMI 1640 or DMEM can be used as a culture medium, and adequate antibiotics or animal serum may be added to such basal medium to conduct culture. Also, a culture vessel is not particularly limited, and a commercialized plate, dish, or flask can be adequately selected in accordance with a scale of culture.

Concentration of GM-CSF, IL-4, IL-10, TGF-β1, TNF-α, or LPS for culture is 1 ng/ml to 1,000 ng/ml, and preferably 10 ng/ml to 100 ng/ml. The number of days necessary for stimulation is not limited. For example, human monocytes may be cultured together with GM-CSF, IL-4, IL-10, TGF-β 1, TNF-α, or LPS for periods of several days to about 10 days. Inspection of expression of human monocytes or human DC surface antigen by FACS or other means enables determination of a suitable culture period for obtaining cells with a differentiation level of interest. Conditions such as concentration of GM-CSF, IL-4, IL-10, TGF-β1, TNF-α, or LPS for stimulation or a period of stimulation can be determined while employing induction of antigen-specific anergy to allogeneic CD4⁺ T cells or DC phenotypes as indicators.

Human immunoregulatory DCs are capable of inducing antigen-specific anergy to allogeneic CD4⁺ T cells in vitro, suppressing reactivation of activated allogeneic CD4⁺ T cells and CD8⁺ T cells, inducing allogeneic and naive CD4⁺ T cells and CD8⁺ T cells to become CD4⁺ CD25⁺ immunoregulatory T cells and CD8⁺ CD28⁻ immunoregulatory T cells, respectively, and inducing immune-suppressing responses such as the suppression of graft-versus-host disease after xenogeneic transplantation in a human T cell-transplanted immunodeficient mouse through administration of the aforementioned cells to which xenoantigens have been imparted. Whether or not cells have such features can be determined by the method described in the Examples below.

These human immunoregulatory DCs exhibit similar functions even when they were treated with inflammatory cytokines such as TNF-α in addition to immature cells. This indicates that these DCs can function sufficiently even under inflammatory disease conditions. Specifically, the immunoregulatory DCs of the present invention include both mature and immature DCs. Whether a DC is mature or immature can be determined by, for example, inspecting the expression of CD83 on the surface thereof. CD83 is expressed on the surface of a mature DC.

2. Use of Human Immunoregulatory DCs

As mentioned above, the human immunoregulatory DCs of the present invention are capable of inducing antigen-specific anergy to allogeneic CD4⁺ T cells in vitro, suppressing reactivation of activated allogeneic CD4⁺ T cells and CD8⁺ T cells, inducing allogeneic and naive CD4⁺′ T cells and CD8⁺ T cells to become CD4⁺ CD25⁺ immunoregulatory T cells and CD8⁺ CD28⁻ immunoregulatory T cells, respectively, and inducing immune-suppressing responses such as the suppression of graft-versus-host disease after xenogeneic transplantation in a human T cell-transplanted immunodeficient mouse through administration of the aforementioned cells to which xenoantigens have been imparted.

As with the case of human immunoregulatory dendritic cells, murine immunoregulatory DCs induced with the aid of IL-10 in combination with TGF-β1 exhibited effects of suppressing graft-versus-host disease after allogeneic transplantation while maintaining the graft-versus-leukemia effects and effects of suppressing the developed disease in a murine autoimmune arthritis model, in addition to the effects of suppressing graft-versus-host disease after allogeneic transplantation and graft-versus-host disease after xenogeneic transplantation. Functions of these murine immunoregulatory DCs are equivalent to those of the aforementioned human immunoregulatory DCs in the following respects, and human dendritic cells were suggested to be effective for treatment of diseases presented in the case of murine immunoregulatory DCs: 1) the phenotype of a cell surface molecule: a costimulating molecule (CD40, CD80, or CD86) being expressed at low frequency and an MHC molecule being expressed; 2) induction of antigen-specific anergy to allogeneic CD4⁺ T cells; 3) suppression of reactivation of activated allogeneic CD4⁺ T cells; 4) suppression of graft-versus-host disease after xenogeneic transplantation in a human T cell-transplanted immunodeficient mouse; 5) induction of CD4⁺ CD25⁺ CD152⁺ T cells in vitro; and 6) the fact that human immunoregulatory DCs induce cells similar to CD4⁺ CD25⁺ immunoregulatory T cells involved in the suppression of graft-versus-host disease after allogeneic transplantation caused by murine immunoregulatory DCs in vitro.

As a conventional technique, immunoregulatory DCs induced with the aid of IL-10 or TGF-β alone had been discovered. The human immunoregulatory DCs of the present invention induced with the aid of IL-10 in combination with TGF-β more significantly induced antigen-specific anergy to allogeneic CD4⁺ T cells compared with DCs induced with the aid of a cytokine alone. This indicates that the human immunoregulatory DCs of the present invention have more significant therapeutic effects compared with those attained by DCs induced with the aid of a cytokine alone.

As is apparent from the foregoing description, human immunoregulatory DCs can suppressively regulate immune responses of CD4⁺ or CD8⁺ T cells. Accordingly, the human immunoregulatory DCs of the present invention can be used for novel treatment of a variety of diseases caused by immune reactions associated with CD4⁺ or CD8⁺ T cells.

Diseases to be treated in the present invention are: in addition to graft rejection caused along with cell, organ, or tissue transplantation and graft-versus-host disease, autoimmune diseases such as chronic rheumatism, multiple sclerosis, type I diabetes, uveitis, autoimmune myocarditis, myasthenia gravis, systemic erythematodes, autoimmune hemolytic anemia, systemic scleroderma, ulcerous colitis, Crohn's disease, Sjogren's syndrome, autoimmune hepatopathy (e.g., primary biliary cirrhosis), psoriasis, idiopathic thrombocytopenic purpura, Goodpasture syndrome (e.g., glomerular nephritis), pernicious anemia, Hashimoto's disease, vitiligo vulgaris, Behcet's disease, autoimmune gastritis, pemphigus, Guillain-Barre syndrome, and HTLV-1-associated myelopathy; and allergic diseases such as contact hypersensitivity, allergic rhinitis, food allergies, and asthma.

The present invention includes a pharmaceutical composition for treating the aforementioned diseases comprising the human immunoregulatory DCs induced by the method of the present invention. When the human immunoregulatory DCs of the present invention are used for treating diseases, they are stimulated with an antigen associated with the disease to be treated. In the case of autoimmune disease or allergic diseases, antigen proteins or peptides existing in tissues or organs associated with the disease, RNA or DNA encoding them, or modified forms thereof are used as antigens. In the case of graft rejection or graft-versus-host disease, application of an antigen is not necessary since DCs have internally expressed allogeneic antigen. Alternatively, a donor- or recipient-derived antigen may be used. As stimulation in such a case, the human immunoregulatory DCs of the present invention may be cultured in vitro together with an antigen.

Human DCs for a pharmaceutical composition for treatment are human monocyte-derived DCs (Bwatrice Thurner, Gerold Schuler et al, J. Exp. Med. 1999, 190 (11): 1669-1678; Axel Heiser, Eli Gilboa el al, J. Clin. Invest. 2002, 109 (3): 409-417), human peripheral blood-derived DCs (Small E J., L Clin Oncol. 2000, 18 (23): 3894-3903), or human CD34⁺ cell-derived DCs (Caux C, Jacques Banchereau et al Blood 1997, 90 (4): 1458-1470), with human monocyte-derived DCs being preferable.

When treating autoimmune or allergic diseases, antigens are imparted to DCs in vitro for 1 to 10 days including the final day of culture at concentrations of 1 to 1,000 μg/ml, and preferably 10 to 100 μg/ml in the case of protein antigens. When human immunoregulatory DCs that were further stimulated with inflammatory stimuli such as TNF-α or LPS are used, antigens are preferably imparted simultaneously with or prior to the application of stimuli.

When a pharmaceutical composition comprising the human immunoregulatory DCs of the present invention is used for treatment, this composition is intravenously, subcutaneously, or intracutaneously (preferably intravenously) administered in amounts of 0.5×10⁶ to 10⁹ in terms of each DC fraction.

The pharmaceutical composition can be administered to a patient on an as-needed basis (preferably during the symptom-free period). In the case of graft rejection and graft-versus-host disease caused along with organ or tissue transplantation, the compound is preferably administered to a patient before the treatment that is supposed to result in the development of disease.

The timing of administration and the dose of human immunoregulatory DCs can be adequately determined in accordance with, for example, the type of disease, the severity of disease, and the conditions of a patient.

The present invention includes a method of treating diseases comprising administration of the human immunoregulatory DCs of the present invention. Diseases to be treated by the present invention are: in addition to graft rejection and graft-versus-host disease caused along with cell, organ, or tissue transplantation, autoimmune diseases such as chronic rheumatism, multiple sclerosis, type I diabetes, uveitis, autoimmune myocarditis, myasthenia gravis, systemic erythematodes, autoimmune hemolytic anemia, systemic scleroderma, ulcerous colitis, Crohn's disease, Sjogren's syndrome, autoimmune hepatopathy (e.g., primary biliary cirrhosis), psoriasis, idiopathic thrombocytopenic purpura, Goodpasture syndrome (e.g., glomerular nephritis), pernicious anemia, Hashimoto's disease, vitiligo vulgaris, Behcet's disease, autoimmune gastritis, pemphigus, Guillain-Barre syndrome, and HTLV-1-associated myelopathy; and allergic diseases such as contact hypersensitivity, allergic rhinitis, food allergies, and asthma. In this case, human immunoregulatory DCs to be administered to a patient may be prepared by stimulating monocytes or DCs of the patient in vitro or stimulating monocytes of DCs of an unrelated individual other than the patient in vitro. The present invention also includes the use of the human immunoregulatory DCs of the present invention for preparing therapeutic agents for the aforementioned diseases.

This description includes part or all of the contents as disclosed in the description and/or drawings of Japanese Patent Application No. 2003-073799, which is a priority document of the present application.

### BRIEF DESCRIPTION OF THE DRAWINGS

FIG. 1A shows phenotypes of modified human DCs.

FIG. 1B shows phenotypes of modified human DCs.

FIG. 1C shows phenotypes of modified human DCs.

FIG. 1D shows phenotypes of modified human DCs.

FIG. 2A shows that, among modified human DCs, IL-10/TGF-β1-induced DCs function as immunoregulatory DCs to induce antigen-specific anergy to human T cells and suppress reactivation of activated T cells.

FIG. 2B shows that, among modified human DCs, IL-10/TGF-β1-induced DCs function as immunoregulatory DCs to induce antigen-specific anergy to human T cells and suppress reactivation of activated T cells.

FIG. 2C shows that, among modified human DCs, IL-10/TGF-β1-induced DCs function as immunoregulatory DCs to induce antigen-specific anergy to human T cells and suppress reactivation of activated T cells.

FIG. 2D shows that, among modified human DCs, IL-10/TGF-β1-induced DCs function as immunoregulatory DCs to induce antigen-specific anergy to human T cells and suppress reactivation of activated T cells.

FIG. 2E shows that, among modified human DCs, IL-10/TGF-β1-induced DCs function as immunoregulatory DCs to induce antigen-specific anergy to human T cells and suppress reactivation of activated T cells.

FIG. 2F shows that, among modified human DCs, IL-10/TGF-β1-induced DCs function as immunoregulatory DCs to induce antigen-specific anergy to human T cells and suppress reactivation of activated T cells.

FIG. 2G shows that, among modified human DCs, IL-10/TGF-β1-induced DCs function as immunoregulatory DCs to induce antigen-specific anergy to human T cells and suppress reactivation of activated T cells.

FIG. 3A shows that human immunoregulatory DCs induce CD4⁺ CD25⁺ human immunoregulatory T cells.

FIG. 3B shows that human immunoregulatory DCs induce CD4⁺ CD25⁺ human immunoregulatory T cells.

FIG. 3C shows that human immunoregulatory DCs induce CD4⁺ CD25⁺ human immunoregulatory T cells.

FIG. 4A shows that human immunoregulatory DCs induce CD8⁺ CD28⁻ human immunoregulatory T cells.

FIG. 4B shows that human immunoregulatory DCs induce CD8⁺ CD28⁻ human immunoregulatory T cells.

FIG. 5A shows that human immunoregulatory DCs suppress graft-versus-host disease after xenogeneic transplantation caused by human T cells.

FIG. 5B shows that human immunoregulatory DCs suppress graft-versus-host disease after xenogeneic transplantation caused by human T cells.

FIG. 6A shows that murine immunoregulatory DCs suppress human xenogeneic T cell responses.

FIG. 6B shows that murine immunoregulatory DCs suppress human xenogeneic T cell responses.

FIG. 6C shows that murine immunoregulatory DCs suppress human xenogeneic T cell responses.

FIG. 6D shows that murine immunoregulatory DCs suppress human xenogeneic T cell responses.

FIG. 6E shows that murine immunoregulatory DCs suppress human xenogeneic T cell responses.

FIG. 7A shows phenotypes of murine immunoregulatory DCs and also shows that murine immunoregulatory DCs induce antigen-specific anergy to murine T cells and suppress reactivation of activated T cells.

FIG. 7B shows phenotypes of murine immunoregulatory DCs and also shows that murine immunoregulatory DCs induce antigen-specific anergy to murine T cells and suppress reactivation of activated T cells.

FIG. 7C shows phenotypes of murine immunoregulatory DCs and also shows that murine immunoregulatory DCs induce antigen-specific anergy to murine T cells and suppress reactivation of activated T cells.

FIG. 7D shows phenotypes of murine immunoregulatory DCs and also shows that murine immunoregulatory DCs induce antigen-specific anergy to murine T cells and suppress reactivation of activated T cells.

FIG. 7E shows phenotypes of murine immunoregulatory DCs and also shows that murine immunoregulatory DCs induce antigen-specific anergy to murine T cells and suppress reactivation of activated T cells.

FIG. 7F shows phenotypes of murine immunoregulatory DCs and also shows that murine immunoregulatory DCs induce antigen-specific anergy to murine T cells and suppress reactivation of activated T cells.

FIG. 7G shows phenotypes of murine immunoregulatory DCs and also shows that murine immunoregulatory DCs induce antigen-specific anergy to murine T cells and suppress reactivation of activated T cells.

FIG. 7H shows phenotypes of murine immunoregulatory DCs and also shows that murine immunoregulatory DCs induce antigen-specific anergy to murine T cells and suppress reactivation of activated T cells.

FIG. 8A shows therapeutic effects of murine immunoregulatory DCs on murine acute graft-versus-host disease after allogeneic transplantation.

FIG. 8B shows therapeutic effects of murine immunoregulatory DCs on murine acute graft-versus-host disease after allogeneic transplantation.

FIG. 8C shows therapeutic effects of murine immunoregulatory DCs on murine acute graft-versus-host disease after allogeneic transplantation.

FIG. 8D shows therapeutic effects of murine immunoregulatory DCs on murine acute graft-versus-host disease after allogeneic transplantation.

FIG. 9A shows the effects of murine immunoregulatory DCs on immune responses of allogeneic marrow graft recipients and the half-lives after administration of DCs in living mice.

FIG. 9B shows the effects of murine immunoregulatory DCs on immune responses of allogeneic marrow graft recipients and the half-lives after administration of DCs in living mice.

FIG. 9C shows the effects of murine immunoregulatory DCs on immune responses of allogeneic marrow graft recipients and the half-lives after administration of DCs in living mice.

FIG. 9D shows the effects of murine immunoregulatory DCs on immune responses of allogeneic marrow graft recipients and the half-lives after administration of DCs in living mice.

FIG. 9E shows the effects of murine immunoregulatory DCs on immune responses of allogeneic marrow graft recipients and the half-lives after administration of DCs in living mice.

FIG. 10A shows the association of murine immunoregulatory T cells with therapeutic effects of murine immunoregulatory DCs for murine acute graft-versus-host disease after allogeneic transplantation.

FIG. 10B shows the association of murine immunoregulatory T cells with therapeutic effects of murine immunoregulatory DCs for murine acute graft-versus-host disease after allogeneic transplantation.

FIG. 10C shows the association of murine immunoregulatory T cells with therapeutic effects of murine immunoregulatory DCs for murine acute graft-versus-host disease after allogeneic transplantation.

FIG. 10D shows the association of murine immunoregulatory T cells with therapeutic effects of murine immunoregulatory DCs for murine acute graft-versus-host disease after allogeneic transplantation.

FIG. 10E shows the association of murine immunoregulatory T cells with therapeutic effects of murine immunoregulatory DCs for murine acute graft-versus-host disease after allogeneic transplantation.

FIG. 10F shows the association of murine immunoregulatory T cells with therapeutic effects of murine immunoregulatory DCs for murine acute graft-versus-host disease after allogeneic transplantation.

FIG. 10G shows the association of murine immunoregulatory T cells with therapeutic effects of murine immunoregulatory DCs for murine acute graft-versus-host disease after allogeneic transplantation.

FIG. 10H shows the association of murine immunoregulatory T cells with therapeutic effects of murine immunoregulatory DCs for murine acute graft-versus-host disease after allogeneic transplantation.

FIG. 10I shows the association of murine immunoregulatory T cells with therapeutic effects of murine immunoregulatory DCs for murine acute graft-versus-host disease after allogeneic transplantation.

FIG. 10J shows the association of murine immunoregulatory T cells with therapeutic effects of murine immunoregulatory DCs for murine acute graft-versus-host disease after allogeneic transplantation.

FIG. 11 shows phenotypes of cells induced in vitro by stimulating CD4⁺ CD25⁻ T cells with murine immunoregulatory DCs.

FIG. 12A shows that murine immunoregulatory DCs suppress murine acute graft-versus-host disease after allogeneic transplantation while maintaining their graft-versus-leukemia effects.

FIG. 12B shows that murine immunoregulatory DCs suppress murine acute graft-versus-host disease after allogeneic transplantation while maintaining their graft-versus-leukemia effects.

FIG. 12C shows that murine immunoregulatory DCs suppress murine acute graft-versus-host disease after allogeneic transplantation while maintaining their graft-versus-leukemia effects.

FIG. 13A shows that murine immunoregulatory DCs suppress murine type II collagen-induced arthritis.

FIG. 13B shows that murine immunoregulatory DCs suppress murine type II collagen-induced arthritis.

### BEST MODES FOR CARRYING OUT THE INVENTION

Examples that describe specific embodiments and effects of the present invention are provided below, although the technical scope of the present invention is not limited to these examples.

### A: EXAMPLES EMPLOYING HUMAN IMMUNOREGULATORY DCs

Three types of modified human DCs, i.e., IL-10-induced DCs, TGF-β1-induced DCs, and IL-10/TGF-β1-induced DCs, were prepared. Based on the results attained in Example 2, IL-10/TGF-β1-induced DCs exhibiting the most potent capacity of regulating T-cell functions were determined to be human immunoregulatory DCs.

### EXAMPLE 1

### Phenotypes of Modified Human DCs

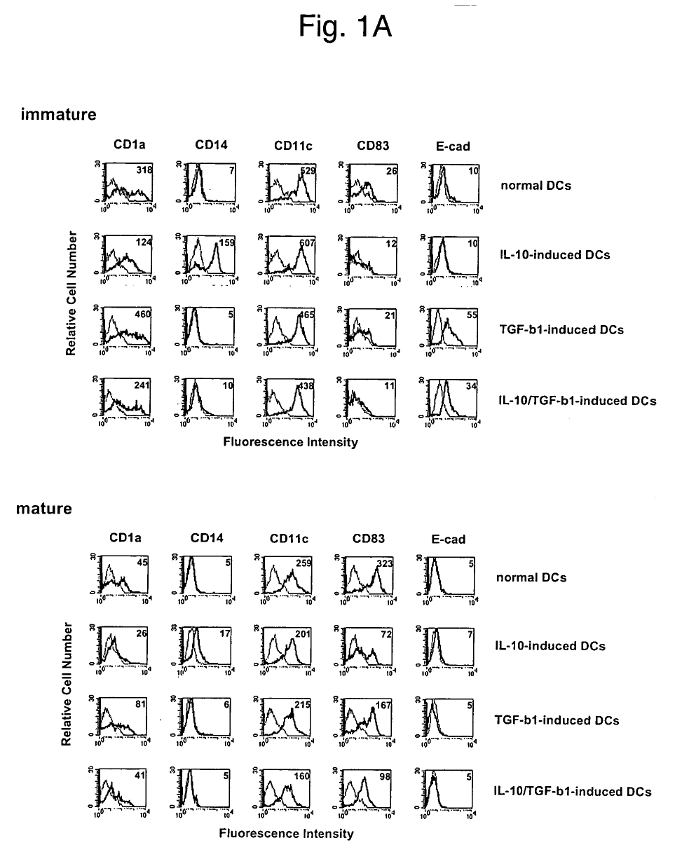

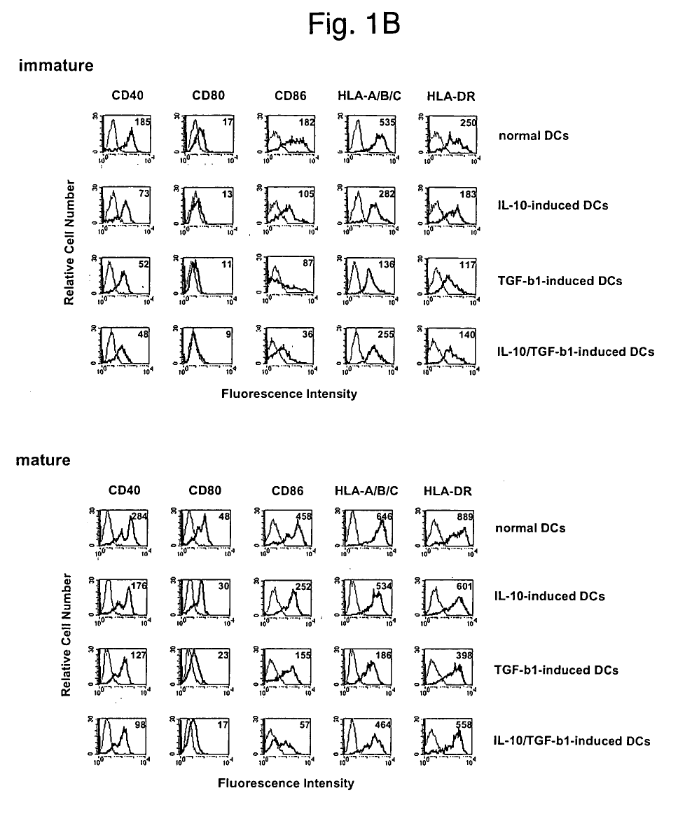

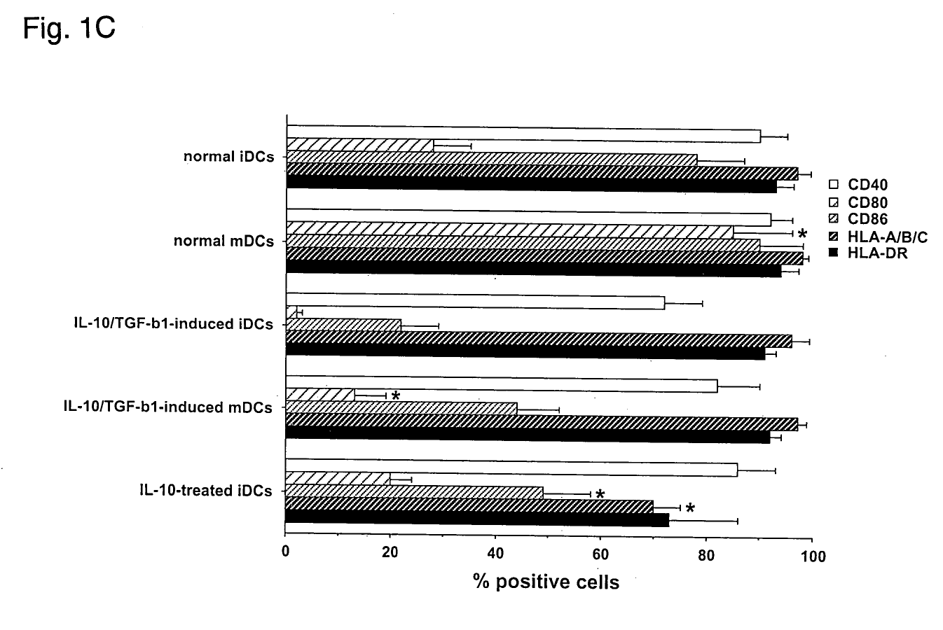

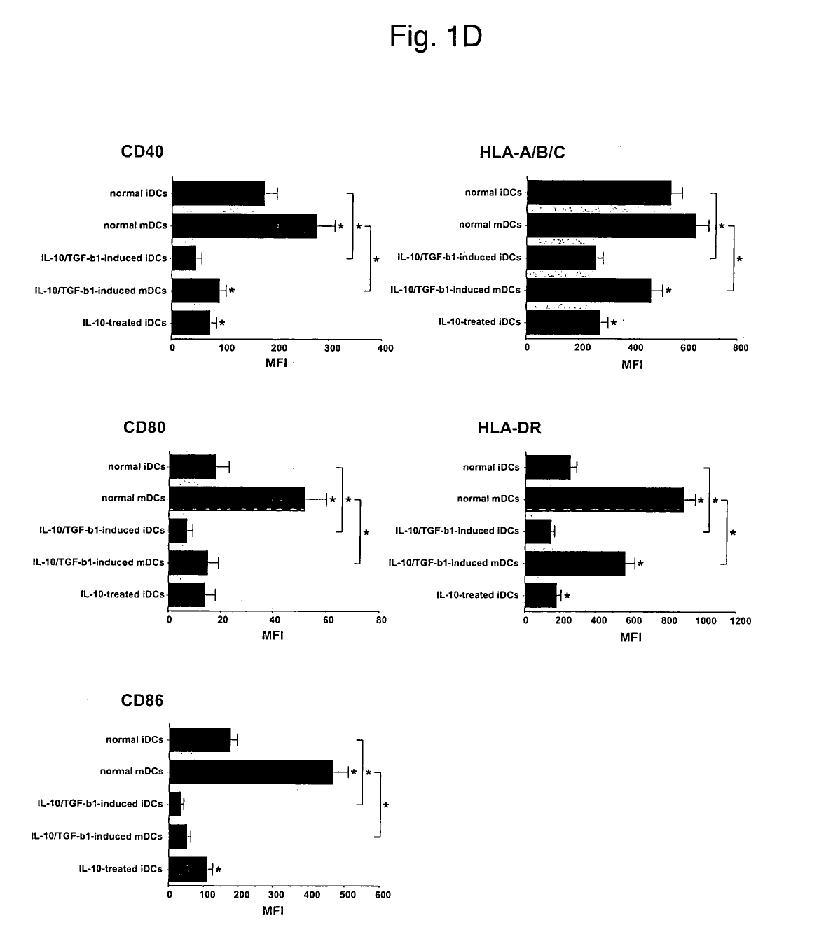

Human DCs were prepared in the following manner. Human peripheral blood-derived mononuclear cells were allowed to adhere to a dish for cell culture (Becton Dickinson) for 2 hours, and monocytes were obtained as adherent cells (>90% CD14⁺ cells). These monocytes were cultured in the presence of human GM-CSF (50 ng/ml, PeproTech) and human IL-4 (50 ng/ml, PeproTech) for 7 days, nonadherent cells were recovered, and negative selection was carried out using an anti-CD2 monoclonal antibody (Dynal) and an anti-CD19 monoclonal antibody (Dynal) to which magnetic beads had been coupled to remove contaminating T cells, NK cells, and B cells. Cells remaining after the removal were determined to be immature normal DCs. Similarly, modified human DCs were prepared by culturing monocytes with human IL-10 (50 ng/ml, PeproTech) alone (IL-10-induced DC), human TGF-β1(50 ng/ml, PeproTech) alone (TGF-β1-induced DC), or human IL-10 (50 ng/ml, PeproTech) in combination with human TGF-β1 (IL-10/TGF-β1-induced DC) in the presence of human GM-CSF (50 ng/ml) and human IL-4 (50 ng/ml) for 7 days, and then removing contaminating T cells, NK cells, and B cells as with the case of the aforementioned immature DCs. Human IL-10-treated immature DCs were obtained by allowing human IL-10 (50 ng/ml, PeproTech) to act on immature DCs similar to the aforementioned DCs for 3 days. Mature DCs were prepared in the following manner. In order to keep the cells from becoming contaminated with cytokines, the aforementioned cells were washed three times with PBS, cultured in the presence of human TNF-α (50 ng/ml, PeproTech) for an additional 3 days, and the resultants were determined to be mature DCs. The obtained DCs were analyzed using the FACScan flow cytometer (Becton Dickinson), 95% or more thereof were found to be HLA-DR-expressing cells, and contamination with T cells, B cells, NK cells, or monocytes/macrophages was not more than 0.1 %. Phenotypes of the thus prepared immature/mature human DCs, immature/mature human IL-10-induced DCs, immature/mature human TGF-β1-induced DCs, and immature/mature human IL-10/TGF-β1-induced DCs were analyzed by a flow cytometer. The results yielded representative data for 10 separate experiments. FIG. 1A shows the results of staining using an anti-CD1a antibody, an anti-CD14 antibody, an anti-CD11c antibody, an anti-CD83 antibody, an anti-E-cad antibody, and isotype controls thereof (BD PharMingen). FIG. 1B shows the results of staining using an anti-CD40 antibody, an anti-CD80 antibody, an anti-CD86 antibody, an anti-HLA/A/B/C antibody, an anti-HLA-DR antibody, and isotype controls thereof (BD PharMingen). Numerical values presented in the upper right of the drawing independently represent mean fluorescence intensity when stained with an antibody. These values indicate that DC markers for all cells, i.e., CD1a and CD11c, were expressed in the case of immature DCs, although the Langerhans cell marker, E-cadherin (E-cad), was not expressed in the IL-10-induced cells as with the case of normal DCs. In the case of IL-10-induced DCs, expression of CD14, which is not observed in other DCs, was observed. Concerning HLA-A/B/C and HLA-DR, a moderate level of expression was observed in IL-10-induced DCs, TGF-β1-induced DCs, and IL-10/TGF-β1-induced DCs, although this expression level was somewhat lower than that in immature normal DCs. Expression levels of CD40, CD80, and CD86 were very low. Concerning DCs that were allowed to mature with the aid of TNF-α, i.e., IL-10/TGF-β1-induced DCs, the expression level of the DC activation marker CD83 was elevated. However, expression levels of the DC markers CD1a and CD11c and the Langerhans cell marker E-cad were lowered. Expression levels of CD83, CD40, CD80, and CD86 were lower in IL-10-induced DCs, TGF-β1-induced DCs, and IL-10/TGF-β1-induced DCs compared with those in mature normal DCs. Expression levels thereof were significantly low particularly in IL-10/TGF-β1-induced DCs. Concerning expressions of HLA and co-stimulatory factors, the ratios of cells to be expressed obtained in 10 separate experiments are presented in terms of mean±SD in FIG. 1C and in terms of MFI±SD in FIG. 1D.

### EXAMPLE 2

### Among Modified Human DCs, IL-10/TGF-β1-Induced DCs Function as Immunoregulatory DCs to Induce Antigen-Specific Anergy to T Cells and Suppress Reactivation of Activated T Cells

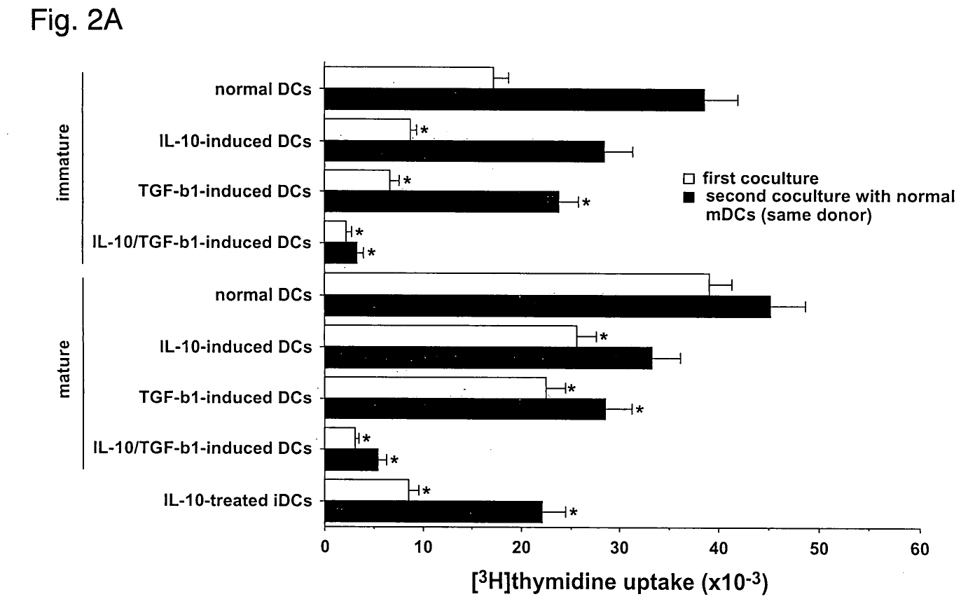

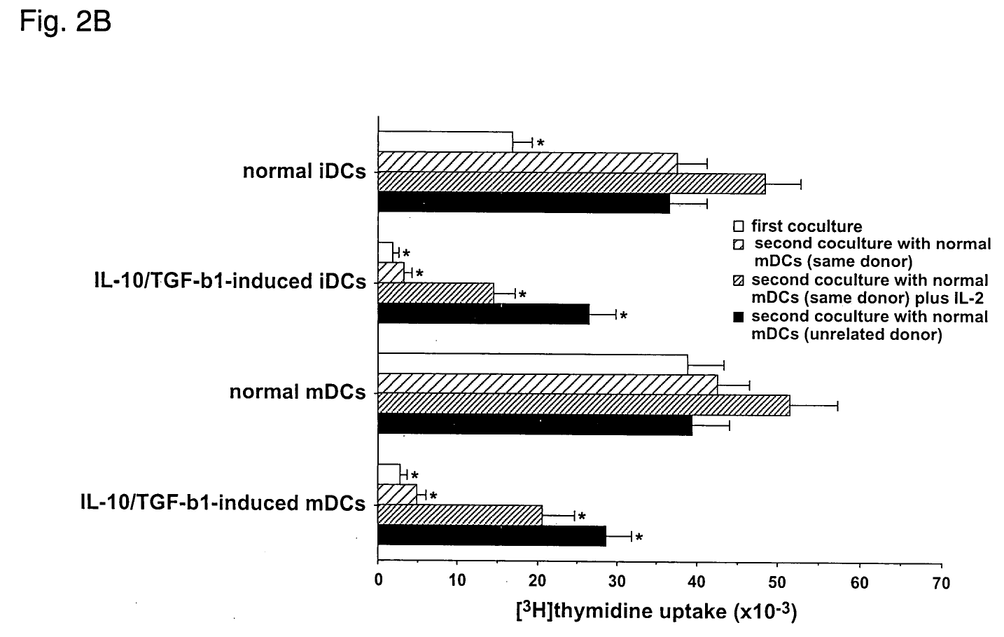

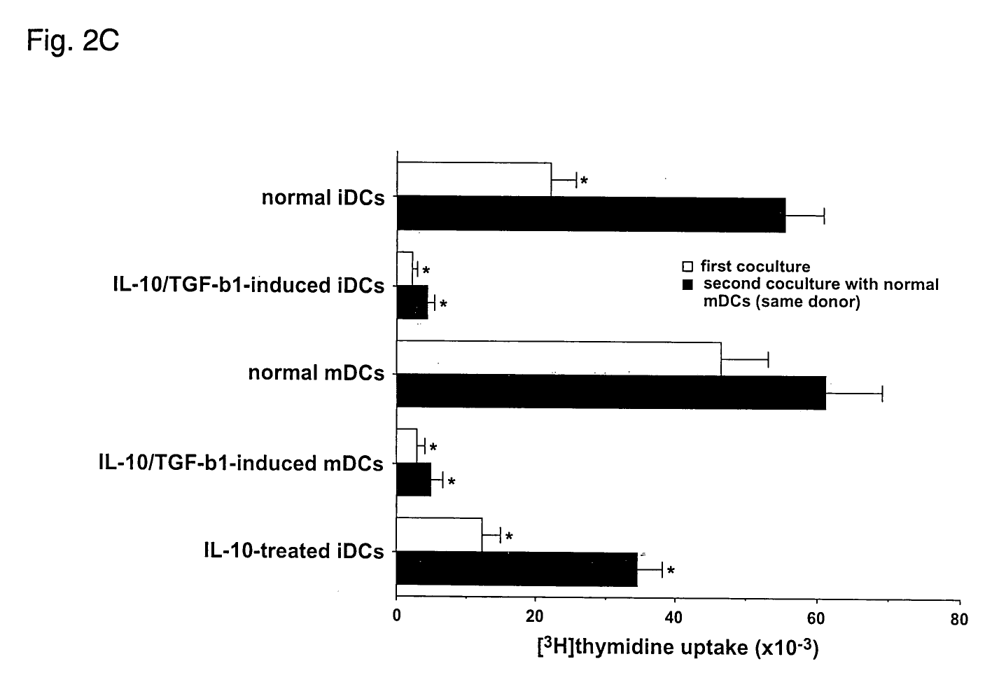

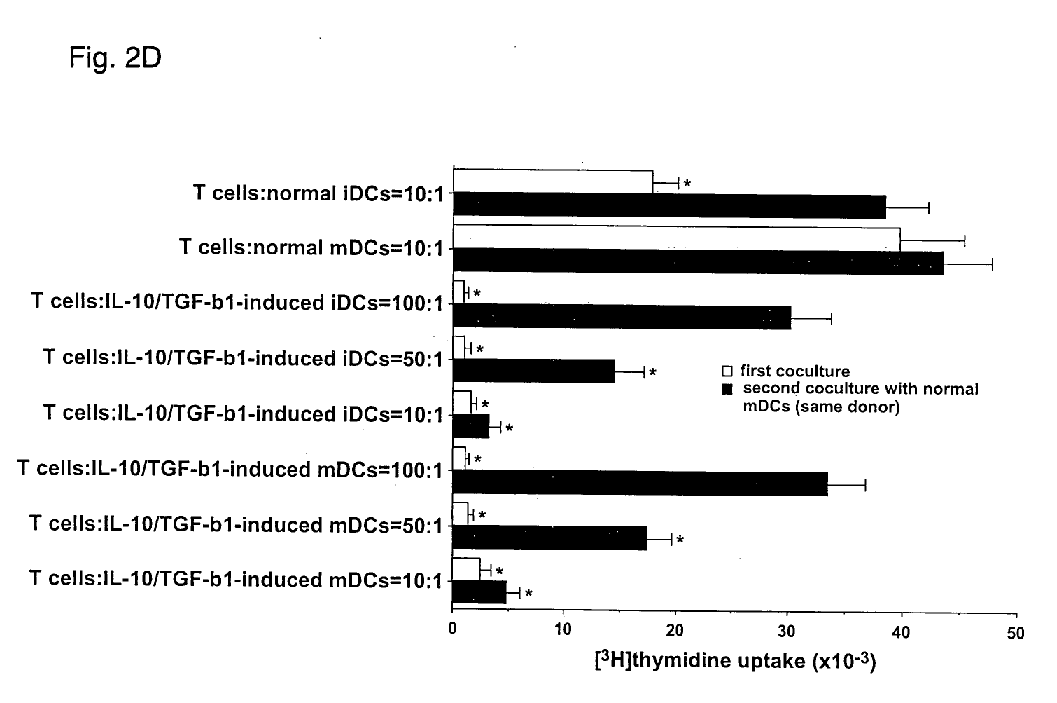

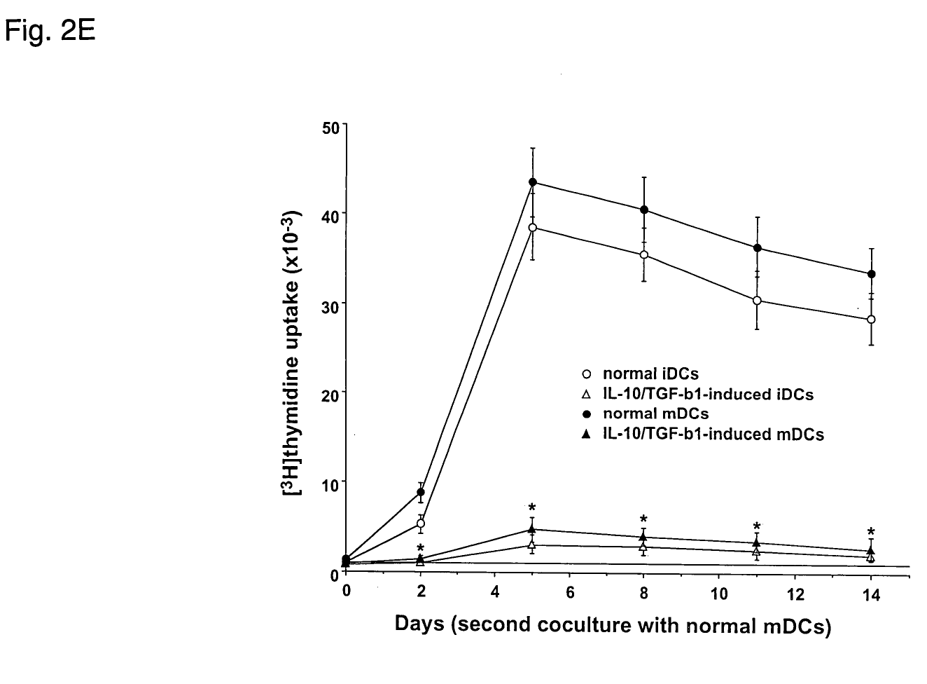

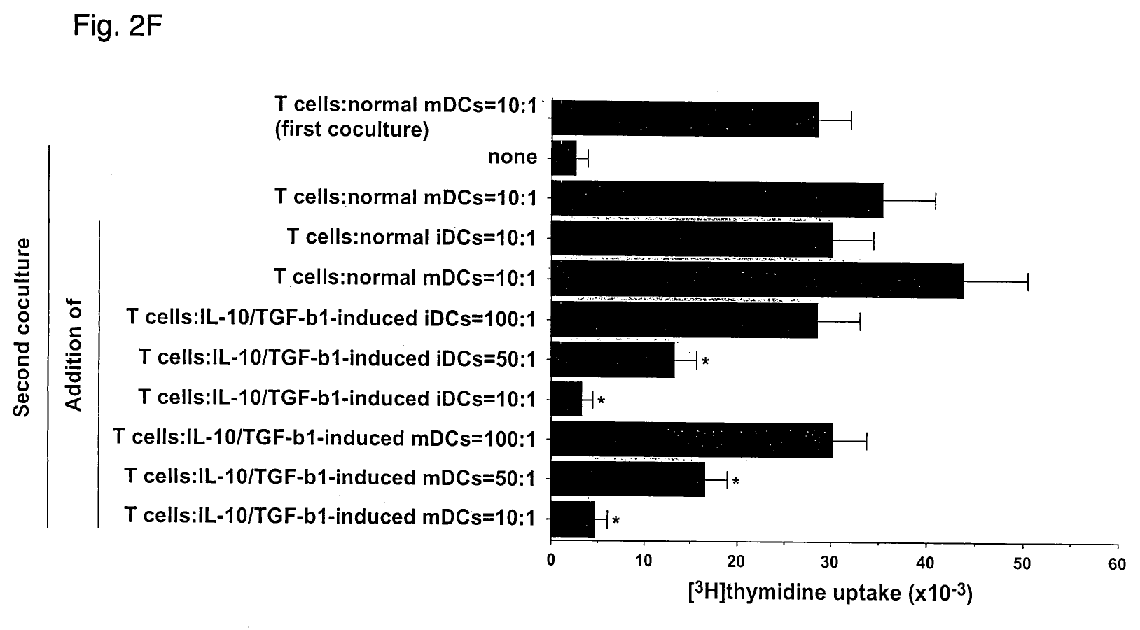

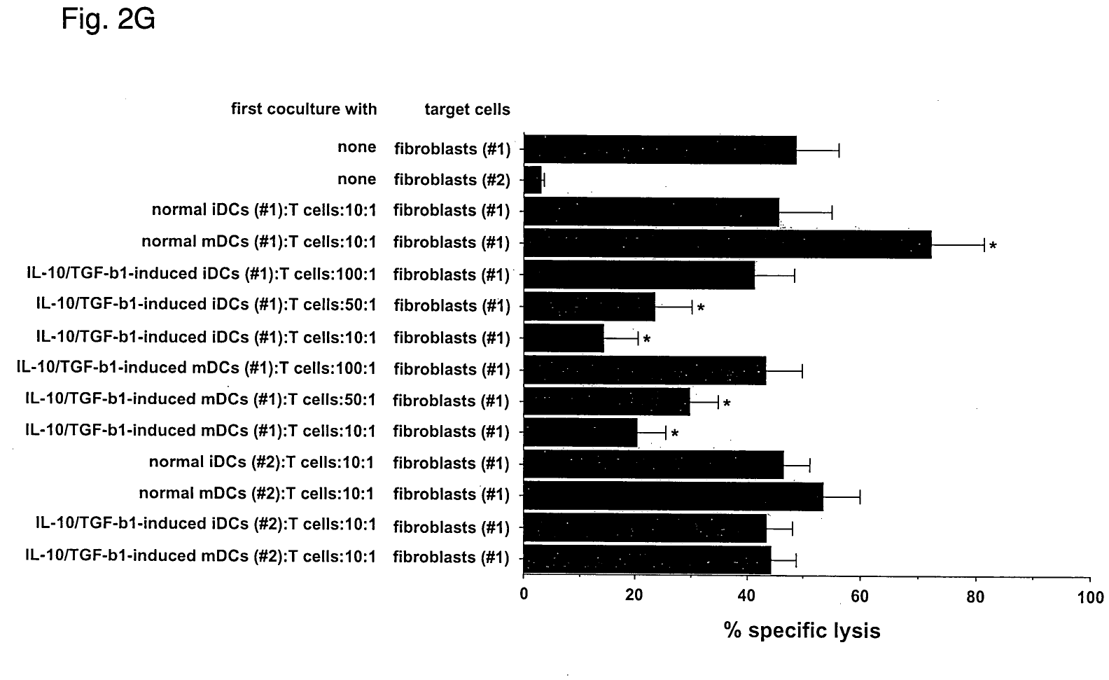

Whether or not modified DCs would induce anergy to allogeneic CD4⁺ T cells was examined. T cells were isolated from human peripheral blood using a negative selection kit (Dynal), and naive CD4⁺ T cells (10⁵ cells), which had been isolated as CD8⁻ CD45RO⁻ cells using an anti-CD8 antibody and an anti-CD45RO antibody (BD PharMingen), were cultured with allogeneic DCs or allogeneic modified DCs (10³ to 10⁴ cells) for 5 days to conduct cell growth assay. In another experiment, naive CD4⁺ T cells (5×10⁶ cells) were cultured with X-ray (15 Gy)-irradiated allogeneic DCs or allogeneic modified DCs (4×10⁴ to 5×10⁵ cells) for 3 days, and negative selection was carried out using an anti-CD11c antibody and a magnetic-beads-coupled goat anti-mouse IgG antibody to recover CD4⁺ cells. The recovered CD4⁺ cells (10⁵ cells) were subjected to the second culture together with allogeneic normal mature human DCs (10⁴ cells) derived from the same donor as in the case of the first stimulation or allogeneic normal mature human DCs (10⁴ cells) derived from a donor different from that of the case of the first stimulation in the presence or absence of IL-2. Cell growth assay was carried out on the fifth day. In the cell growth assay, cells were pulsed with [³H]thymidine for 18 hours, and incorporation of [³H]thymidine into cells was employed as an indicator. As shown in FIG. 2A, assay was carried out using IL-10-induced DC, TGF-β1-induced DC, IL-10/TGF-β1-induced DC, or IL-10-treated DC. In the case of immature DCs, the capacity of modified DCs to activate allogeneic CD4⁺ T cells at the time of primary stimulation was uniformly low, and that of immature IL-10/TGF-β1-induced DCs was the lowest. When mature DCs were used, however, the capacity of IL-10/TGF-β1-induced DCs to activate allogeneic CD4⁺ T cells at the time of primary stimulation was significantly lower than that of other mature DCs. When primarily stimulated with each of the DCs and then secondarily stimulated with mature normal DCs, growth was suppressed only in the both immature and mature IL-10/TGF-β1-induced DCs. In the case of IL-10-treated immature DCs, potent suppressing activities were observed in both experiments for primary and secondary stimulations, although this suppressing activity was not as potent as that of IL-10/TGF-β1-induced DCs. In FIG. 2B, suppressing activity tended to cease with the addition of IL-2 at the time of secondary stimulation after stimulation with IL-10/TGF-β1-induced DCs. When mature normal DCs derived from an unrelated donor were used for secondary stimulation, the level of suppression was insignificant. This indicates that IL-10/TGF-β1-induced DCs induce anergy to naive CD4⁺ cells in an antigen-specific manner. Similar results were observed when all of the CD4⁺ T cells were used as responders, and the level of suppression was more potent than that of the IL-10-treated immature DCs (FIG. 2C). The suppression effects attained at the time of secondary stimulation with mature normal DCs were exhibited in a manner dependent on the dosage of the IL-10/TGF-β1-induced DCs added at the time of primary stimulation (FIG. 2D). Changes in cell growth after secondary stimulation were inspected with the elapse of time, and cell growth suppressing effects were maintained for at least two weeks (FIG. 2E). Further, the activity of IL-10/TGF-β1-induced DCs upon naive CD4⁺ cells that had been activated by mature allogeneic DCs was examined. This revealed that IL-10/TGF-β1-induced DCs suppressed the cell growth in a dose-dependent manner (FIG. 2F). Subsequently, the activity of IL-10- and TGF-β1-induced DCs upon the cytotoxicity of antigen-specific CD8⁺ T cells was examined (FIG. 2G). T cells were isolated from human peripheral blood using a negative selection kit (Dynal), and naive CD8⁺ T cells were isolated as CD4⁻ and CD45RO⁻ cells using an anti-CD4 antibody and an anti-CD45RO antibody (BD PharMingen). Antigen-specific CD8⁺ T cells were obtained by culturing X-ray (15 Gy)-irradiated allogeneic fibroblasts (donor #1) and PBMC for 2 weeks (100 U/ml of IL-2 added) and subjecting CD8⁺ T cells to positive selection. The antigen-specific CD8⁺ cells were cultured in the presence or absence of allogeneic DCs (donor #1 or #2) for 3 days. Cytotoxicity assay was carried out by culturing the CD8⁺ T cells (5×10⁵ cells) with allogeneic fibroblasts labeled with Na₂⁵¹CrO₄ (100 μCi/10⁶ cells, NEN™ Life Science Product, Boston, Mass.) (donor #1 or #2) for 4 hours and assaying the radioactivity of the culture supernatant. As a result, CD8⁺ T cells were found to exhibit cytotoxicity in a manner specific to the allogeneic fibroblasts (donor #1) used for stimulation, i.e., in an antigen-specific manner. In this case, cytotoxicity was enhanced when stimulation took place with mature normal DCs instead of immature normal DCs. In contrast, IL-10/TGF-β1-induced DCs suppressed cytotoxicity in a dose-dependent manner. When unrelated donor-derived DCs were used, cytotoxicity was not substantially affected. Specifically, immunoregulatory activity caused by the IL-10/TGF-β1-induced DCs was considered to be specific. Accordingly, IL-10/TGF-β1-induced DCs were found to be capable of regulating activities of all effector T cells. The IL-10/TGF-β1-induced DCs are hereafter referred to as “immunoregulatory DCs.”

### EXAMPLE 3

### Human Immunoregulatory DCs Induce CD4⁺ CD25⁺ Immunoregulatory T Cells

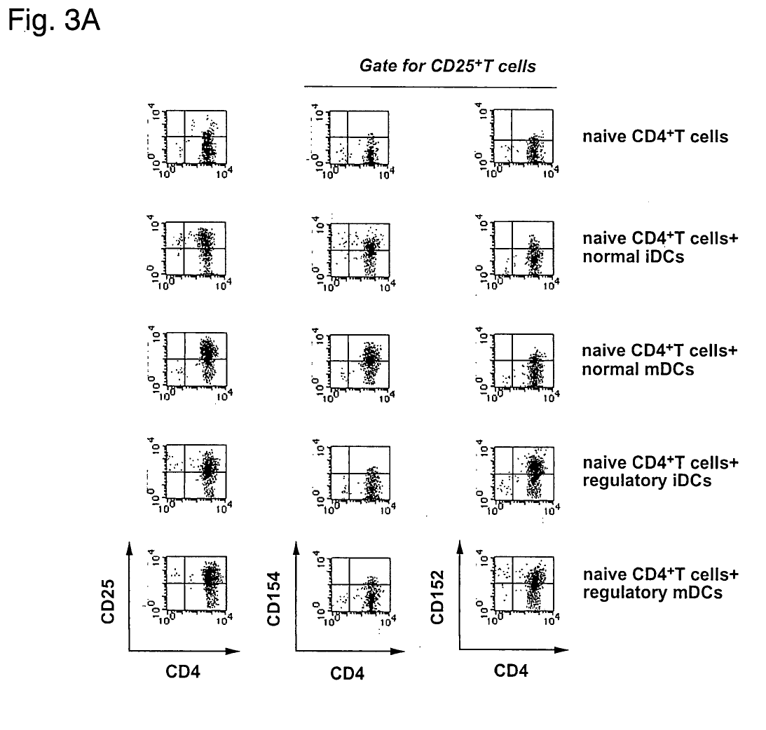

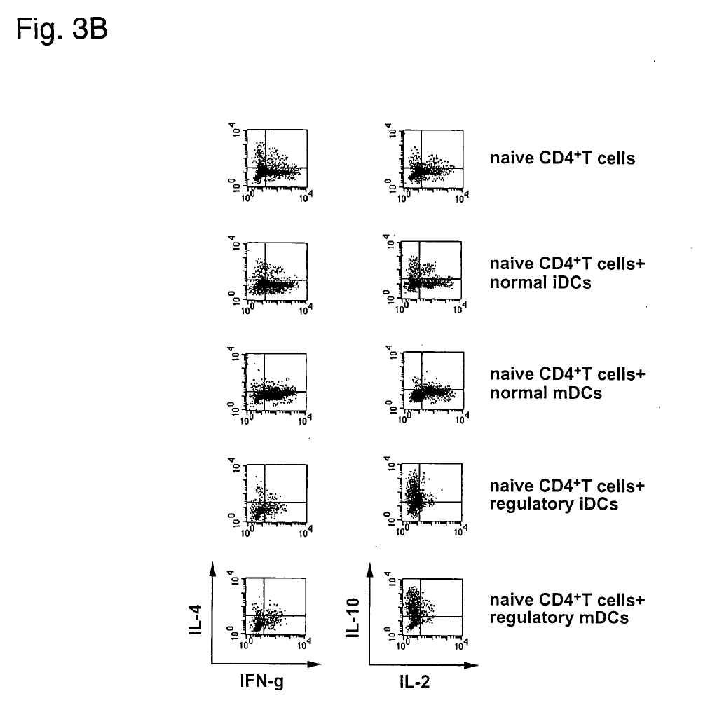

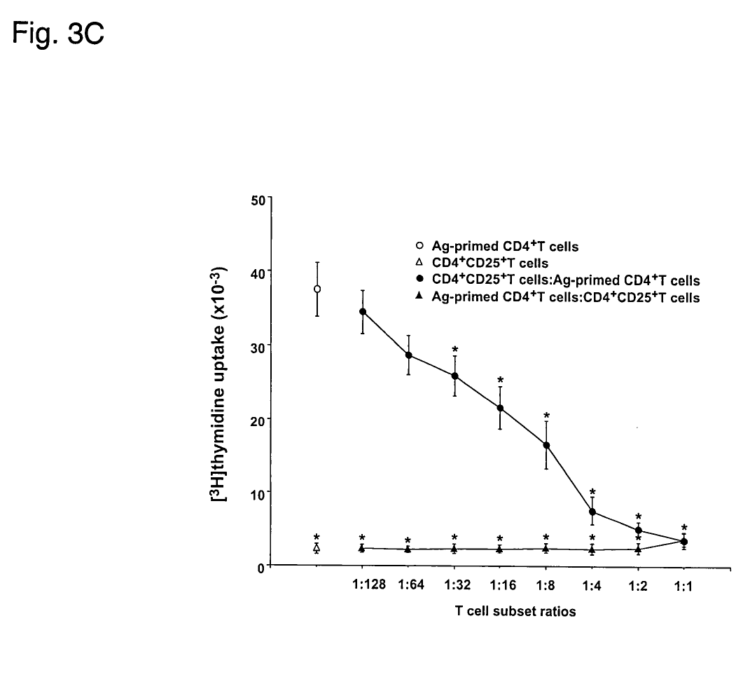

Human naive CD4⁺ T cells (5×10⁶ cells) isolated in a manner equivalent to that of Example 2 were cultured together with allogeneic DCs or allogeneic immunoregulatory DCs (5×10⁵ cells) for 5 days. The obtained T cells were analyzed for cell surface antigens and intracellular cytokines using FACS. Intracellular cytokine production was analyzed in the following manner. Cells were stimulated with an anti-human CD3 antibody immobilized on a plate (10 μg/ml, BD PharMingen) and with a solubilized anti-human CD28 antibody (10 μg/ml, BD PharMingen) for 6 hours. The resulting cells were permeated, immobilized, and then stained with anti-human IL-2, IL-4, IL-10, and interferon (IFN)-γ (BD PharMingen) for analysis using FACS. The results represent one typical data set attained in 5 separate experiments. When allogeneic normal DCs were used, CD4⁺ CD25⁺ cells and CD4⁺ CD154⁺ cells were induced. In contrast, CD4⁺ CD25⁺ cells and CD4⁺ CD152⁺ cells were induced when allogeneic immunoregulatory DCs were used (FIG. 3A). Concerning intracellular cytokine production, IFN-γ- and IL-2-producing cells increased when stimulated with allogeneic normal DCs whereas IL-10-producing cells increased when stimulated with allogeneic immunoregulatory DCs (FIG. 3B). Further, functions of CD4⁺ CD25⁺ T cells induced with the aid of allogeneic immunoregulatory DCs were analyzed (FIG. 3C). The method was as described below. Human naive CD4⁺ T cells (5×10⁶ cells) were cultured together with X-ray (15 Gy)-irradiated allogeneic murine normal mature DCs (5×10⁵ cells) for 3 days, negative selection was carried out using an anti-CD11c antibody and a magnetic-beads-coupled goat anti-mouse IgG antibody, and antigen-stimulated CD4⁺ cells were obtained. Human CD4⁺ CD25⁺ T cells were isolated by culturing naive CD4⁺ T cells (5×10⁶ cells) with allogeneic immature immunoregulatory DCs (5×10⁵ cells) for 5 days and using an anti-CD25 antibody (BD PharMingen) and a magnetic-beads-coupled goat anti-mouse IgG antibody (15 Gy). As a result of FACS analysis, purity was found to be 95% or higher. The obtained antigen-stimulated CD4⁺ cells were mixed with a different amount of CD4⁺ CD25⁺ T cells, the resultant was cultured with allogeneic mature normal DCs (10⁴ cells) for an additional 5 days, and cell growth was then assayed. While the antigen-stimulated CD4⁺ cells alone responded to allogeneic mature normal DCs and abundantly grew, CD4⁺ CD25⁺ T cells alone did not substantially responded thereto. When CD4⁺ CD25⁺ T cells were cultured together with antigen-stimulated CD4⁺ cells, they suppressed the growth of stimulated CD4⁺ cells in a dose-dependent manner. However, the suppression effects were not dissolved even though the number of CD4⁺ CD25⁺ T cells was maintained at a constant level while the number of antigen-stimulated CD4⁺ cells was increased. This indicates that cell growth is not merely suppressed by competitive inhibition against allogeneic antigens. This suppression effect disappears when the CD4⁺ CD25⁺ T cells are separated from the stimulated CD4⁺ cells in a transwell, and the suppression activity partially disappears with the addition of IL-2. An IL-10- or TGF-β-neutralizing antibody did not affect the suppression activity (data is not shown). In the case of CD4⁺ CD25⁺ T cells (donor A, induced by immunoregulatory DCs of allogeneic mice B), suppression of the activation of naive CD4⁺ T cells (donor A) by allogeneic mature normal DCs (donor B) is twice as potent as suppression of the activation of naive CD4⁺ T cells (donor A) by allogeneic mature normal DCs (donor C). This indicates that the suppression activity by CD4⁺ CD25⁺ T cells can be antigen-specific or non-specific (data is not shown). Accordingly, immunoregulatory DCs effectively induce CD4⁺ CD25⁺ immunoregulatory T cells.

### EXAMPLE 4

### Human Immunoregulatory DCs Induce CD8⁺ CD28⁻ Immunoregulatory T Cells

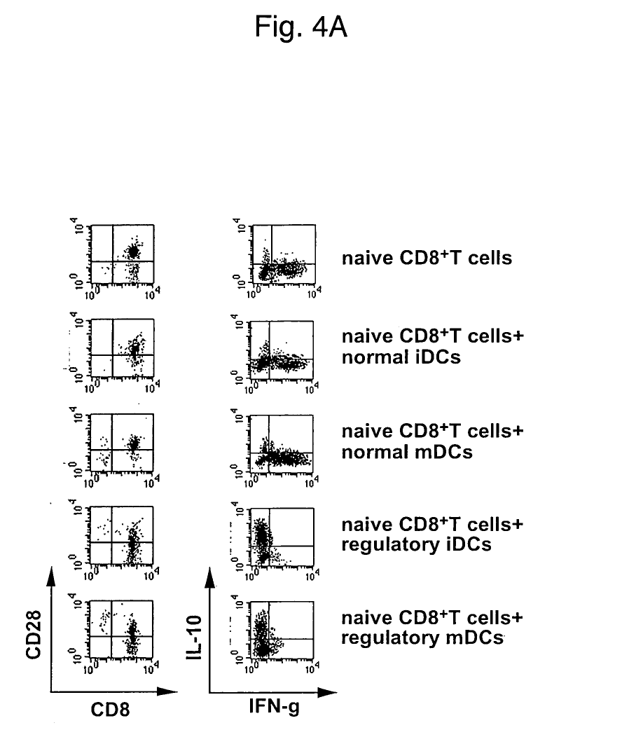

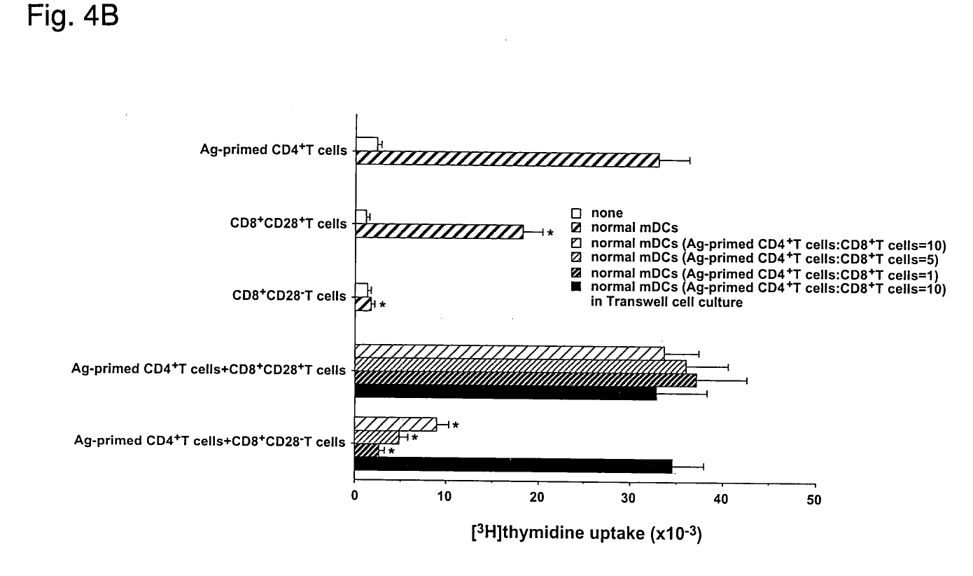

Human naive CD8⁺ T cells (5×10⁶ cells) isolated in a manner similar to that of Example 2 were cultured together with X-ray (15 Gy)-irradiated allogeneic normal DCs or allogeneic immunoregulatory DCs (5×10⁵ cells) for 5 days, and negative selection was carried out using an anti-CD11c antibody and a magnetic-beads-coupled goat anti-mouse IgG antibody to recover CD8⁺ T cells. These cells were subjected to analysis of cell surface antigens (left in FIG. 4A) and inspection of intracellular cytokine production. Cell surface antigens were analyzed in accordance with Example 1, and intracellular cytokines were assayed in the following manner. Cells stimulated with PMA (20 ng/ml, Sigma) and Ca²⁺ ionophore A23187 (500 ng/ml, Sigma) for 6 hours were permeated, immobilized, stained with anti-human IL-10 and IFN-γ, and then analyzed using FACS. The analysis revealed that CD8⁺ CD28⁺ cells were induced when naive CD8⁺ T cells were cultured together with allogeneic normal DCs whereas CD8⁺ CD28⁻ cells were induced when naive CD8⁺ T cells were cultured together with allogeneic immunoregulatory DCs (FIG. 4A). When intracellular cytokines were inspected, the number of INF-γ-producing cells increased when naive CD8⁺ T cells were cultured together with allogeneic normal DCs. In contrast, when naive CD8⁺ T cells were cultured together with allogeneic immunoregulatory DCs, the number of IL-10-producing cells increased (FIG. 4A). Further, functions of CD8⁺ CD28⁺ cells obtained by culturing with allogeneic mature DCs in the aforementioned manner and those of CD8⁺ CD28⁻ cells obtained by culturing with allogeneic immature immunoregulatory DCs were analyzed. CD8⁺ CD28⁺ T cells or CD8⁺ CD28⁻ T cells (10⁴ to 10⁵ cells) were cultured together with X-ray-irradiated allogeneic mature normal DCs (10⁴ cells) and antigen-stimulated CD4⁺ T cells (10⁵ cells) prepared in the same manner as in Example 3, and cell growth assay was carried out 5 days thereafter. In a transwell experiment utilizing a 24-well plate, X-ray-irradiated allogeneic mature normal DCs (10⁵ cells) were added to CD8⁺ CD28⁺ T cells or CD8⁺ CD28^{-T cells (}10 ⁶ cells), antigen-stimulated CD4⁺ T cells (10⁵ cells) and X-ray-irradiated allogeneic murine normal mature DCs (10⁵ cells) were directly added thereto or partitioned in separate transwell chambers, and the resultant was cultured for 5 days. DCs were removed in the same manner as in Example 2 five days thereafter, T cells (10⁵ cells) were transferred to a 96-well plate, and cell growth assay was carried out. When CD8⁺ CD28⁺ T cells were cultured together with allogeneic mature normal DCs or when antigen-stimulated CD4⁺ T cells were cultured together with allogeneic mature normal DCs, CD4⁺ or CD8⁺ CD28⁺ T cells was found to have been grown. In contrast, CD8⁺ CD28⁻ T cells did not substantially grow when CD8⁺ CD28⁻ T cells were cultured together with allogeneic murine normal mature DCs (FIG. 4B). Further, CD8⁺ CD28⁻ T cells suppressed the growth of CD4⁺ T cells caused by allogeneic mature normal DCs in a dose-dependent manner (FIG. 4B). Transwell isolation experiment revealed that contact between CD4⁺ T cells and CD8⁺ CD28⁻ cells was necessary for this suppression activity (FIG. 4B). This demonstrates that immunoregulatory DCs induce CD8⁺ CD28⁻ immunoregulatory T cells from naive CD8⁺ T cells.

### EXAMPLE 5

### Human Immunoregulatory DCs Suppress Graft-Versus-Host Disease After Xenogeneic Transplantation Caused by Human T Cells

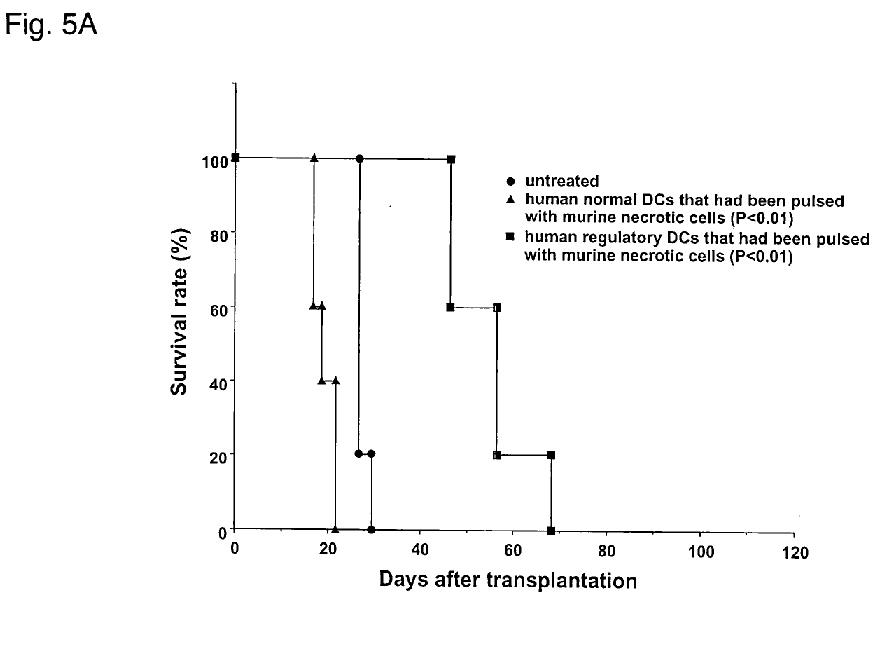

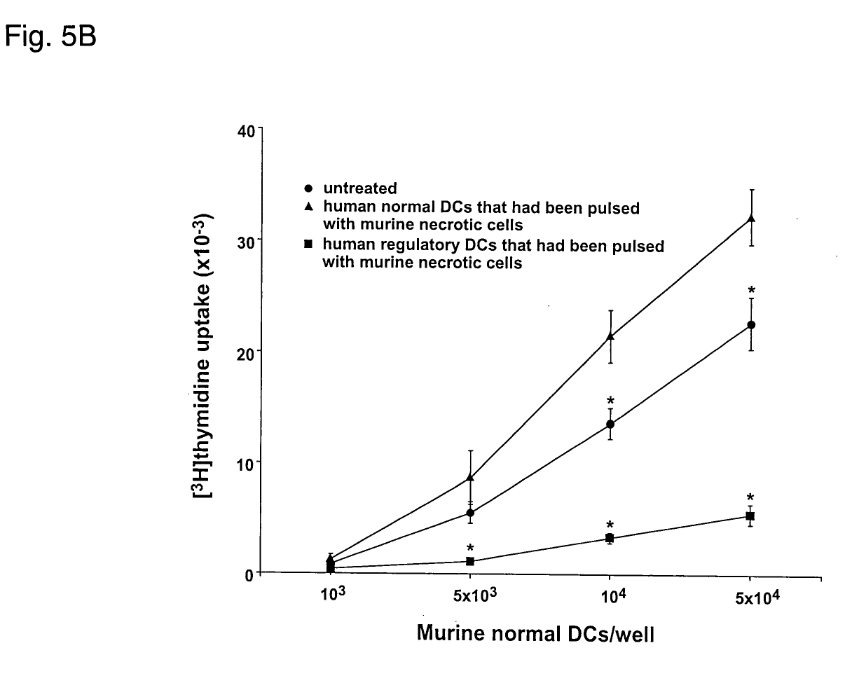

Effects of human immunoregulatory DCs in models of graft-versus-host disease (GvHD) after xenogeneic transplantation (the process is described in Example 7) were examined. Human immunoregulatory DCs were induced in the same manner as in Example 1. Normal immature human DCs or immature human immunoregulatory DCs were pulsed with necrotized spleen cells of BALB/c mice (10⁵ cells) for 24 hours, culture was carried out in the presence of TNF-α (50 ng/ml) for 3 days, and cultured cells were then allowed to mature. Necrotized cells were prepared by subjecting cells to a freeze/thaw cycle four times. Xenogeneic GvHD responses were induced in the same manner as described in Example 7, and the aforementioned cells (4×10⁶ cells) were administered through caudal veins 2 days after the induction. As a result, the mice died due to administration of normal mature human DCs significantly earlier than the control group, and administration of mature human immunoregulatory DCs significantly prolonged their survival (FIG. 5A). In the same manner as in Example 6, human T cells were separated from spleen cells 10 days after administration, and reactivity with murine normal mature DCs was assayed. As a result, human T cells derived from mice to which normal mature human DCs had been administered exhibited significantly higher reactivity compared with that of the control group, and human immunoregulatory T cells derived from mice to which murine normal mature DCs had been administered exhibited significantly lower reactivity compared with that of the control group (FIG. 5B).

### B: EXAMPLES EMPLOYING MURINE IMMUNOREGULATORY DCS

### EXAMPLE 6

### Method of Preparing Murine DCs and Murine T Cells, Method of Stimulating T Cells In Vitro, and Method of Experimentation Utilizing T Cells

Murine normal mature DCs (mDCs) were prepared by culturing bone marrow cells obtained from BALB/c, C57BL/6, DBA/1, or CBA/1 mice in a plastic culture dish in the presence of recombinant murine GM-CSF (20 ng/ml, PeproTech, London, England) for 6 days, and conducting further culture in the presence of LPS (1 μg/ml, Sigma, St. Louis, Mo.) for 2 days. Murine immunoregulatory DCs (rDCs) were prepared by culturing murine bone marrow cells obtained from mice of the same strain as the murine normal mature DCs in a plastic culture dish in the presence of recombinant murine GM-CSF (20 ng/ml, PeproTech, London, England), recombinant murine IL-10 (20 ng/ml, PeproTech, London, England), and recombinant human TGF-β1 (20 ng/ml, PeproTech, London, England) for 6 days, and then conducting further culture in the presence of LPS (1 μg/ml, Sigma, St. Louis, Mo.) for 2 days. Dihydroxyvitamin D₃-stimulated DCs were prepared by culturing murine bone marrow cells obtained from mice of the same strain as the murine normal mature DCs in a culture dish in the presence of recombinant murine GM-CSF (20 ng/ml, PeproTech, London, England) and dihydroxyvitamin D₃ (10 nM, Sigma, St. Louis, Mo.) for 6 days, and then conducting further culture in the presence of LPS (1 μg/ml, Sigma, St. Louis, Mo.) for 2 days.

T cell fractions were prepared in the following manner. Specifically, spleen mononuclear cells fractions of normal mice (H-2^{d}, H-2^{b}, or H-2^{k}) were suspended in PBS, rat antibodies against Ly-76, B220, Ly-6G, and I-A/I-E (BD PharMingen, San Diego, Calif.) were added, incubation was carried out at 4° C. for 30 minutes, cells were washed with PBS, sheep anti-rat IgG mAb-conjugated immunomagnetic beads (Dynal, Oslo, Norway) were added, incubation was carried out again at 4° C. for 30 minutes, cells were washed with PBS, and T cell fractions were obtained by negative selection. A rat anti-CD8 or CD4 antibody (BD PharMingen, San Diego, Calif.) and sheep anti-rat IgG mAb-conjugated immunomagnetic beads were added to aforementioned T cell fractions in the same manner described above, and CD4⁺ T cell fractions or CD8⁺ T cell fractions were obtained by negative selection. As a result of analysis using a flow cytometer FACScan (Becton Dickinson, Mountain View, Calif.), purities of these T cell fractions were all 97% or higher.

Bone marrow cells (1.5×10⁷ cells/mouse) and spleen mononuclear cells (1.5×10⁷ cells/mouse) derived from allogeneic donor mice were administered intravenously to recipient mice to which lethal doses of X-rays had been applied (10 Gy/mouse) and then transplanted. Combinations of a recipient mouse with a donor mouse were: 1) BALB/c(H-2^{d}) with C57BL/6(H-2^{b}); 2) C57BL/6(H-2^{b}) with BALB/c(H-2^{d}); or 3) DBA/1(H-2^{q}) with BALB/c(H-2^{d}). Spleen mononuclear cells were recovered 5 days after transplantation, recipient cells were removed by negative selection using an anti-recipient type I-K rat antibody and anti-rat IgG microbeads to prepare donor-derived spleen mononuclear cells fractions. The yield of this fraction was 2×10⁷ cells/mouse or lower, and the content of donor type I-K⁺ cell was 95% or higher. CD4⁺ T cell fractions and CD8⁺ T cell fractions were prepared in the same manner as in Example 6, and the yield thereof was 4×10⁶ cells/mouse.

Similarly, donor-derived CD4⁺ T cells and CD8⁺ T cells were recovered from the spleen mononuclear cell fraction of recipient mice to which allogeneic transplantation had been applied and murine normal mature DCs or murine immunoregulatory DCs had been then administered. The yield thereof was 1×10⁷ or 3×10⁷ cells/mouse or lower. The recovered cells were cultured in the presence of recombinant murine IL-2 (10 ng/ml) for 3 days and then used in the assay.

### EXAMPLE 7

### Murine Immunoregulatory DCs Suppress Human Xenogeneic T Cell Responses

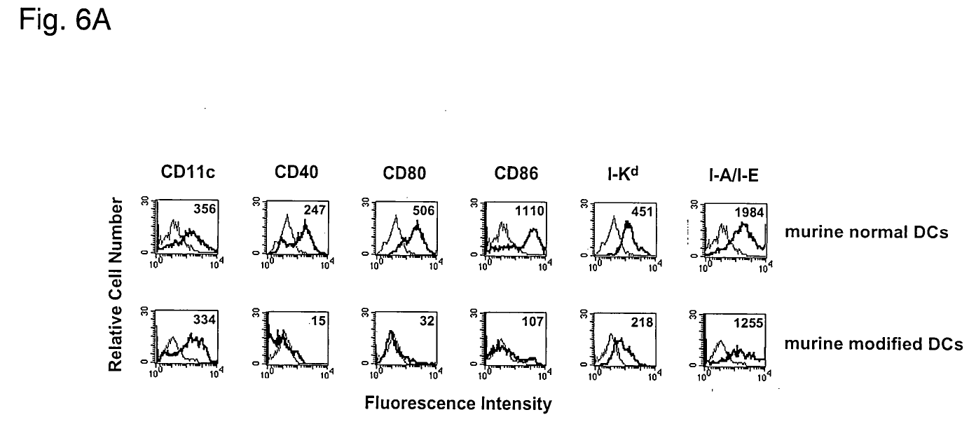

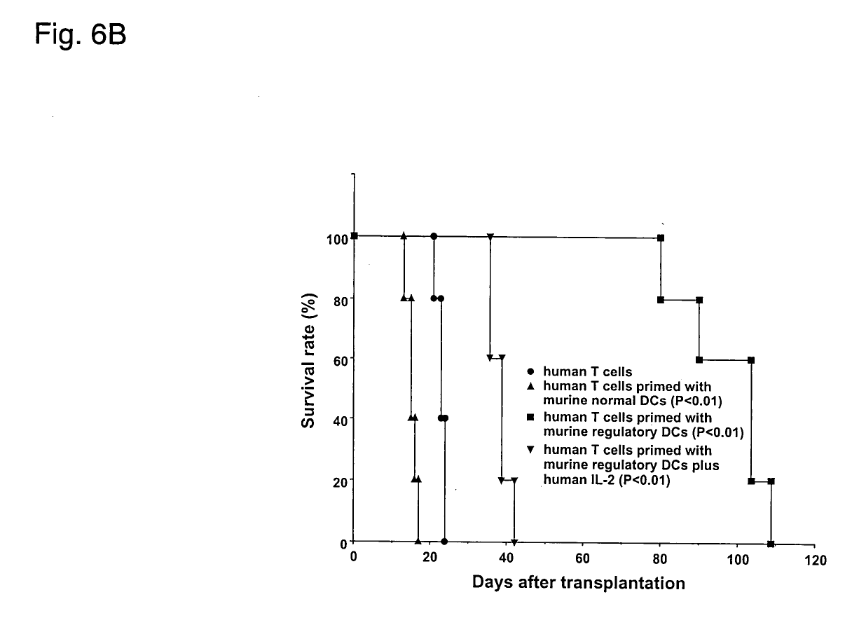

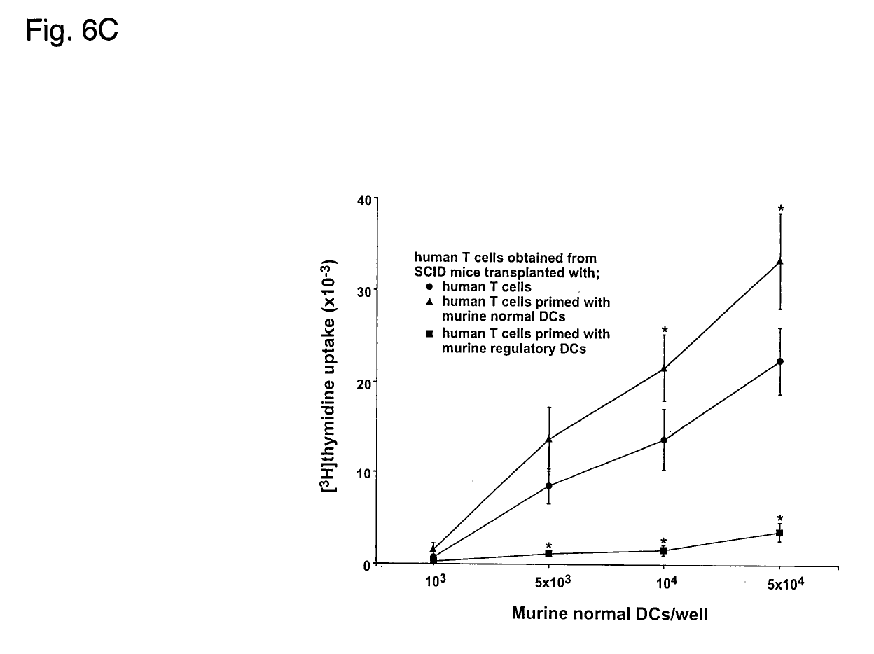

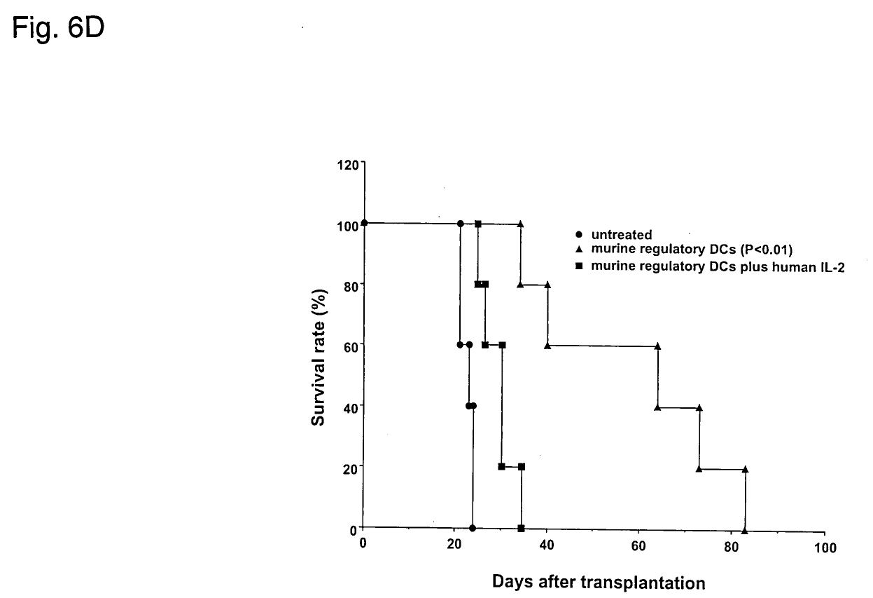

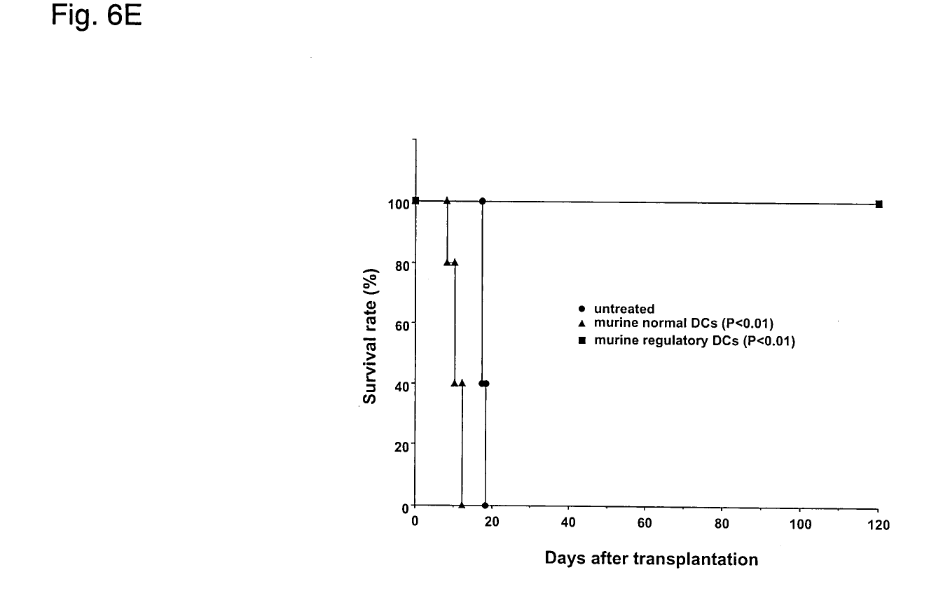

Murine normal mature DCs and murine immunoregulatory DCs were prepared in the same manner as in Example 6. Phenotypes were analyzed using a flow cytometer. An anti-CD11c antibody, an anti-CD40 antibody, an anti-CD80 antibody, an anti-CD86 antibody, an anti-I-K^{d} antibody, an anti-I-A/I-E antibody, and isotype controls thereof (BD PharMingen) were used for staining. Numerical values presented in the upper right of FIG. 6A independently represent mean fluorescence intensity when stained with an antibody. As a result, significant difference was not observed in the expression of I-A/I-K molecules in the case of murine modified DCs compared with the case of murine normal mature DCs, although expression levels of CD40, CD80, and CD86 (costimulating molecules) were lower in murine modified DCs.

The capacity for activating human T cells was examined. This demonstrated that murine immunoregulatory DCs had lower capacity for activating human T cells compared with murine normal mature DCs (data is not shown). While human T cells that had been activated by murine normal mature DCs were reactivated by murine normal mature DCs, human T cells that had been activated by murine immunoregulatory DCs were not reactivated by murine normal mature DCs (data is not shown).

Functions of murine immunoregulatory DCs in xenogeneic GvHD were further examined. Xenogeneic GvHD was induced in the following manner. PBL (5×10⁷ cells) were cultured together with X-ray (15 Gy)-irradiated murine normal mature DCs or murine immunoregulatory DCs (H-2^{d}) (5×10⁶ cells) in the presence or absence of human IL-2 (100 U/ml) for 3 days, negative selection for human T cells was carried out using an anti-I-K^{d} antibody (BD PharMingen) and goat anti-mouse IgG antibody-conjugated immunomagnetic beads, culture was further carried out in the presence of human IL-2 (10 U/ml) for 3 days, and human T cells stimulated with xenoantigens were obtained. Anti-asialo GM1 antiserum (20 μl, 10 mg/ml, Wako Pure Chemical Industries, Ltd.) was administered to C.B.-17-scid recipient mice (H-2^{d}) one day before cell transplantation, and a sublethal dose of X-rays (5 Gy) was applied on the day of cell transplantation. Subsequently, human T cells stimulated with xenoantigens or unstimulated human T cells (4×10⁷ cells) were administered to mice intraveneously. Two days after the administration of human T cells, murine normal mature DCs (4×10⁶ cells) or murine immunoregulatory DCs (4×10⁶ cells) obtained in the same manner as in Example 6 were administered. The group to which murine immunoregulatory DCs and human IL-2 (10⁴ U) were to be simultaneously administered on days 3, 5, and 7 was also provided. As a result, mice (the control group) to which human T cells had been transplanted died within 24 days after the cell transplantation due to xenogeneic GvHD responses. When human T cells stimulated with murine normal mature DCs were transplanted to mice, they died significantly earlier than those in the control group (P<0.01). In contrast, mice survived longer when human T cells stimulated with murine immunoregulatory DCs had been transplanted. When human T cells stimulated with murine immunoregulatory DCs had been transplanted to mice in the presence of IL-2 (100 U/ml), however, the survival of mice was curtailed (FIG. 6B). Ten days after the transplantation, human T cells were recovered from spleen cells of the recipient mice, and their reactivity with murine normal mature DCs was examined in the following manner. Mononuclear cells were recovered from spleen cells using a HISTOPAQUE-1080 (Sigma), negative selection was carried out using an anti-I-K^{b} antibody and goat anti-mouse IgG antibody-conjugated immunomagnetic beads to prepare human T cells, and the resultant was cultured in the presence of human IL-2 (10 U/ml) for 3 days. These human T cells (10⁵ cells) were cultured together with normal mature murine DCs (10³ to 5×10⁴ cells) for 5 days, and cell growth assay was carried out. The assay revealed that human T cells derived from mice to which human T cells stimulated with murine normal mature DCs had been transplanted had higher reactivity with murine normal mature DCs than the control group to which human T cells only had been transplanted (FIG. 6C). In contrast, human T cells derived from mice to which human T cells stimulated with murine immunoregulatory DCs had been transplanted exhibited low reactivity with murine normal mature DCs (FIG. 6C). Survival of the xenogeneic GvHD models was prolonged when murine immunoregulatory DCs had been administered after human T cell transplantation. This effect of prolonged survival was abrogated by IL-2 administration (FIG. 6D).

In order to analyze therapeutic effects of murine immunoregulatory DCs on acute GvHD caused by allogenic bone marrow transplantation utilizing SCID mice, the influence of a single administration of murine immunoregulatory DCs (H-2^{d}) on lethal GvHD, which developed in the recipient mice (H-2^{d}) to which allogeneic bone marrow cells and spleen mononuclear cells (H-2^{b}) had been transplanted, was inspected. The SCID mice (H-2^{d}) were systemically irradiated with a lethal dose of radioactive rays (10 Gy/mouse), and C57BL/6 mice-derived bone marrow cells (H-2^{b}, 2×10⁷ cells/mouse) and G57BL/6 mice-derived spleen mononuclear cells (H-2^{b}, 2×10⁷ cells/mouse) prepared in the manner described above were administered. Two days later, a group to which murine normal mature DCs (4×10⁶ cells/mouse) or murine immunoregulatory DCs (4×10⁶ cells/mouse) prepared in the manner described above were to be administered and a group to which DCs were not to be administered were provided, and the survival (%) of these groups thereafter was observed. All samples in the group to which DCs had not been administered died within 18 days after the transplantation, and all samples in the group to which murine normal mature DCs had been administered died earlier than those in the former group (within 12 days). However, all samples in the group to which murine immunoregulatory DCs had been administered were still alive 120 days after the transplantation. This demonstrates that murine immunoregulatory DCs have therapeutic effects on acute graft-versus-host disease that is developed after allogeneic bone marrow transplantation using SCID mice (FIG. 6E).

### EXAMPLE 8

### Comparison of Cell Surface Molecule Expression of Murine Immunoregulatory DCs With That of Murine Normal Mature DCs and Functions of Murine Immunoregulatory DCs

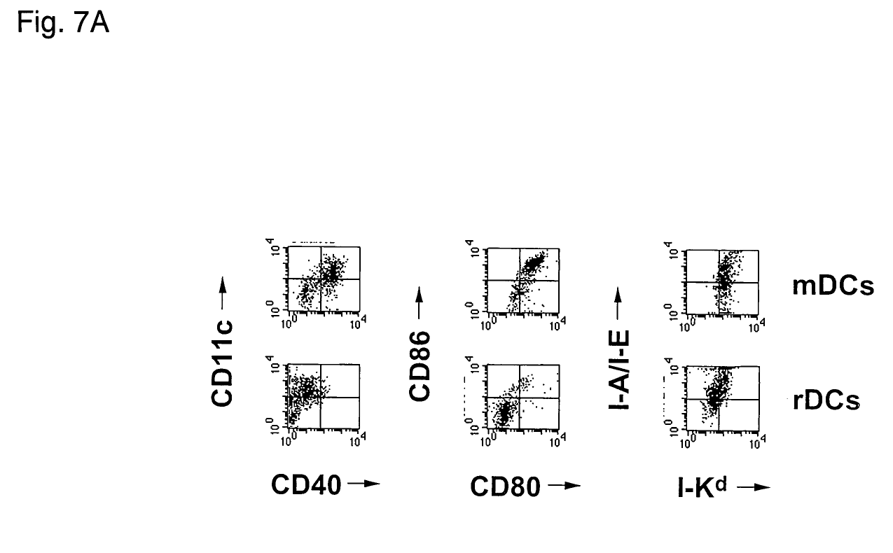

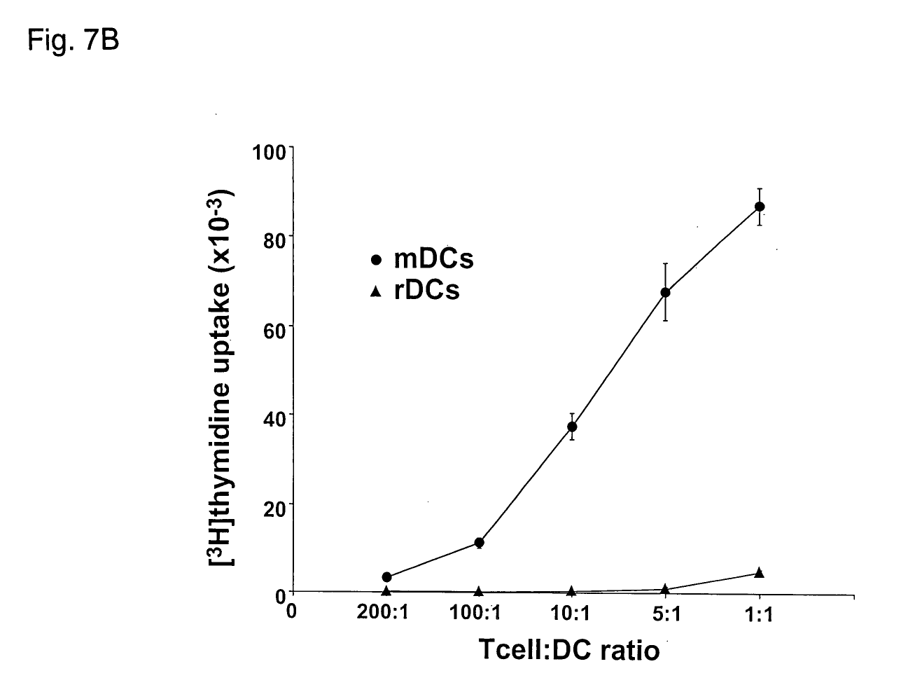

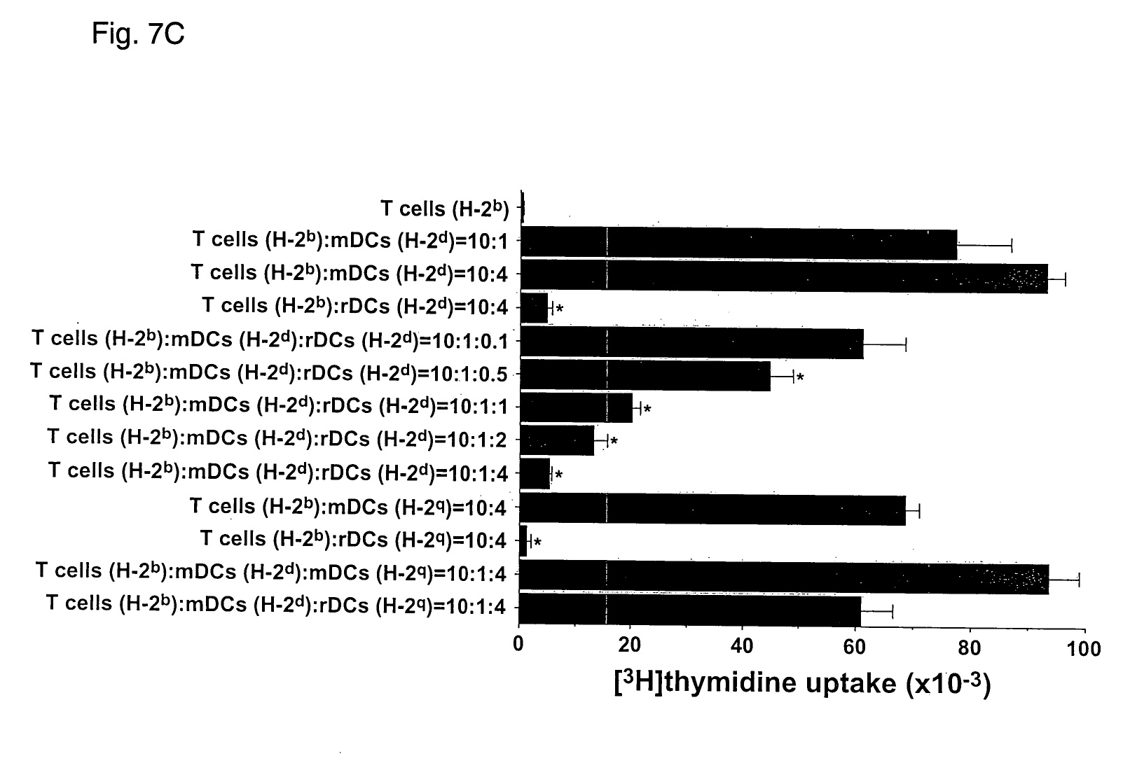

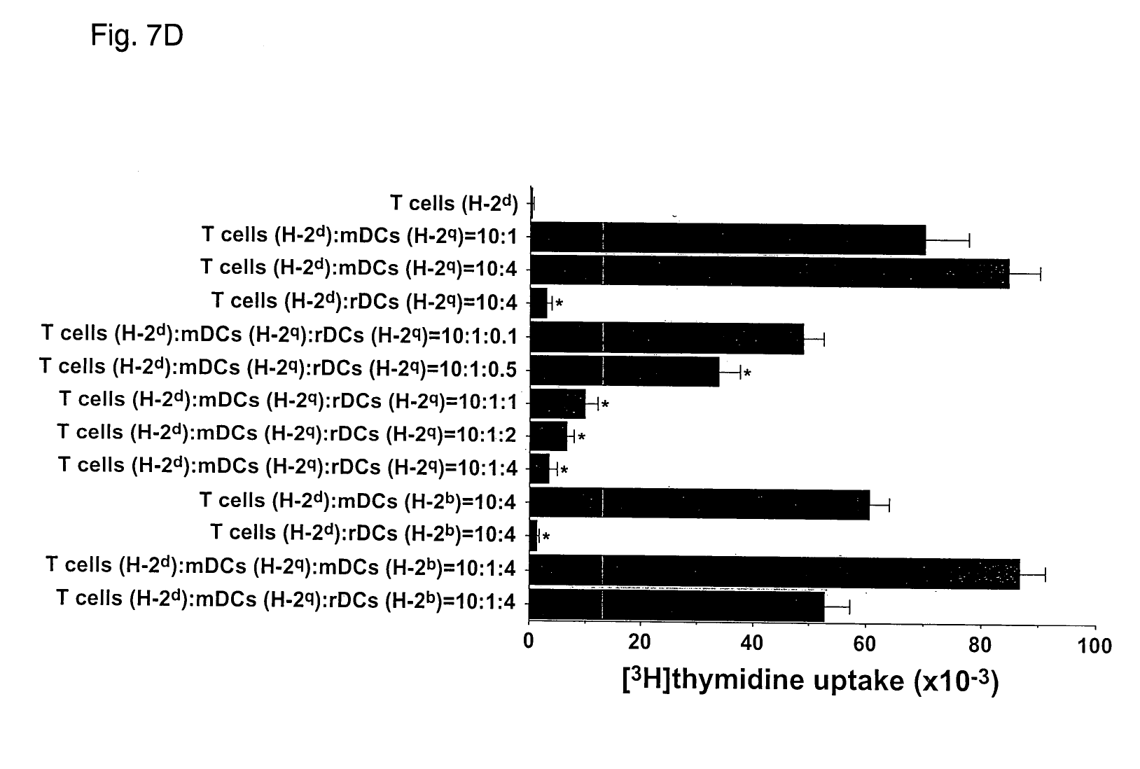

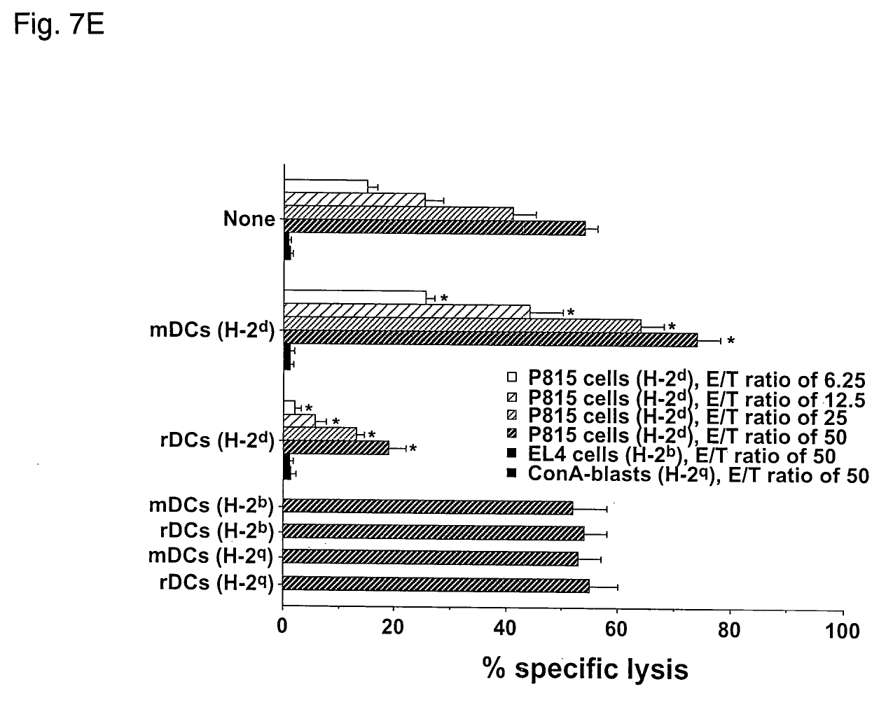

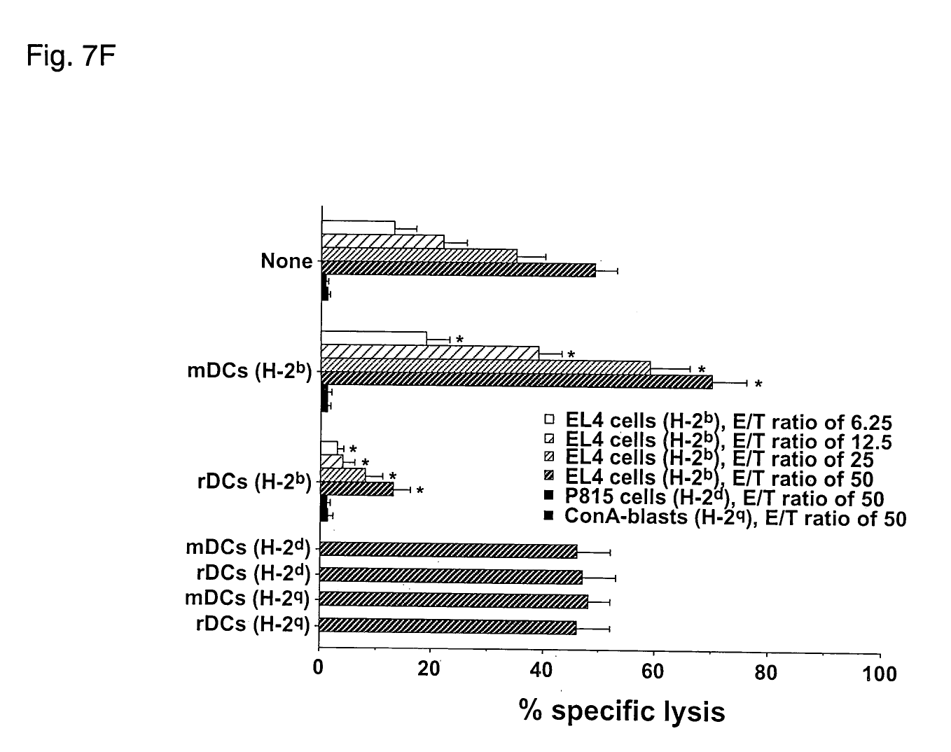

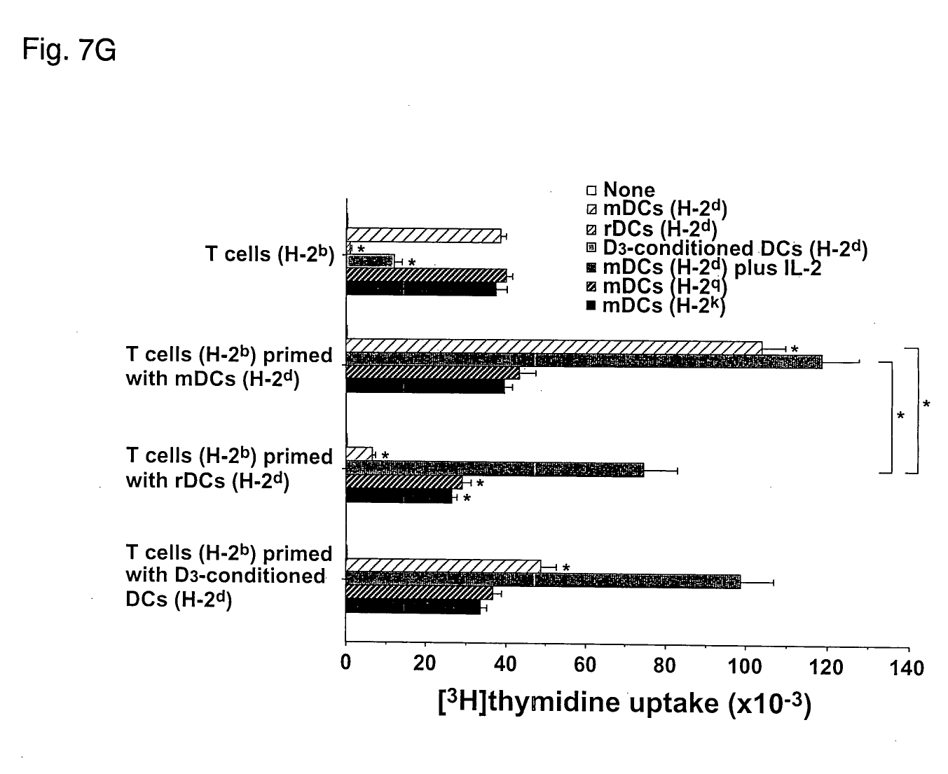

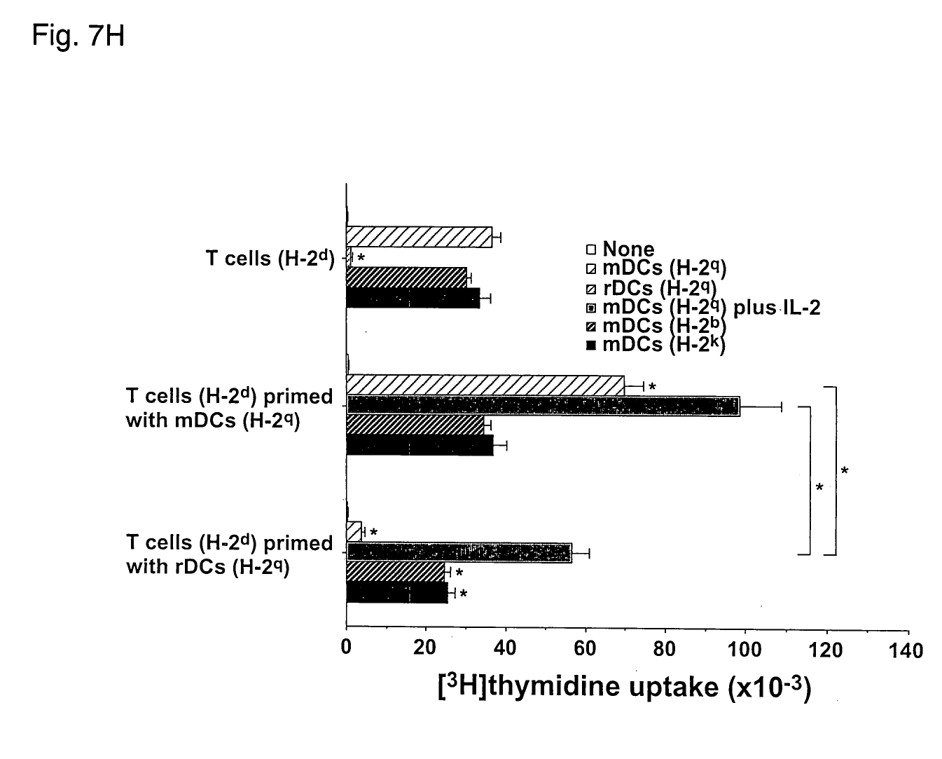

Murine normal mature DCs or murine immunoregulatory DCs prepared in accordance with the method described in Example 6 were washed with PBS, fluorescein-conjugated antibodies specific to DC markers (CD11c), costimulating molecules (CD40, CD80, and CD86), or MHC molecules (I-K^{d} and I-A/I-E) were added, and incubation was then carried out under ice cooling for 30 minutes. After washing with PBS, the expression intensity of each molecule was inspected using a flow cytometer (Becton Dickinson). In murine normal mature DCs (H-2^{d}), CD11c, CD40, CD80, CD86, I-K^{d}, and I-A/I-E were expressed with high intensity and high frequency. In murine immunoregulatory DCs, CD11c and MHC molecules were expressed with high frequency, although expression levels of CD40, CD80, and CD86 were significantly low (Table 1 and FIG. 7A). Expression patterns of cell surface molecules for murine immunoregulatory DCs derived from all the examined mouse strains (H-2^{d}, H-2^{b}, and H-2^{q}) exhibited the same inclination (Table 1).

TABLE 1
Phenotypes and capacity for stimulating allogeneic T cells of immunoregulatory DCs induced from a variety of mouse strains
Phenotypes and allogeneic T-cell capacity of rDcs

| | | | | | | | | Allogeneic T-cell stimulatory capacity (cpm) | | |
|---|---|---|---|---|---|---|---|---|---|---|
| Mouse | | mean % postive cells ± SD/MFI ± SD | | | | | | T cell (H-2^{k})^{a}/DC ratio | | |
| Strain | Type of DCs | CD40 | CD11c | CD80 | CD86 | I-K | I-A and/or I-E | 10:1 | 100:1 | 200:1 |
| BALB/c | mDCs | 68 ± 8/ | 58 ± 7/ | 84 ± 8/ | 77 ± 9/ | 86 ± 10/ | 77 ± 12/ | 39547 ± 2414 | 15474 ± 1252 | 4321 ± 341 |
| mice | | 215 ± 45 | 252 ± 33 | 440 ± 53 | 994 ± 74 | 640 ± 75 | 2405 ± 324 | | | |
| (H-2^{d}) | | | | | | | | | | |
| (n = 10) | rDCs | 4 ± 2/ | 60 ± 5/ | 5 ± 3/ | 10 ± 3/ | 48 ± 10/ | 73 ± 11/ | 541 ± 121 | 194 ± 64 | 154 ± 18 |
| | | 14 ± 3 | 243 ± 41 | 23 ± 11 | 36 ± 12 | 359 ± 45 | 1162 ± 187 | | | |
| | Dⱼ- | 32 ± 5/ | 47 ± 5/ | 29 ± 6/ | 28 ± 7/ | 42 ± 5/ | 48 ± 6/ | 12654 ± 554 | 3354 ± 654 | 1145 ± 221 |
| | conditioned | 130 ± 15 | 178 ± 24 | 111 ± 28 | 257 ± 36 | 284 ± 43 | 687 ± 85 | | | |
| | DCs | | | | | | | | | |
| C57/BL6 | mDCs | 65 ± 7/ | 61 ± 5/ | 80 ± 10/ | 75 ± 8/ | 83 ± 12/ | 75 ± 14/ | 35784 ± 1987 | 13421 ± 1754 | 3982 ± 405 |
| mice | | 204 ± 34 | 294 ± 41 | 387 ± 49 | 979 ± 68 | 628 ± 64 | 2302 ± 287 | | | |
| (H-2^{b}) | | | | | | | | | | |
| (n = 10) | rDCs | 3 ± 2/ | 54 ± 4/ | 4 ± 2/ | 8 ± 3/ | 47 ± 8/ | 74 ± 12/ | 334 ± 84 | 144 ± 34 | 139 ± 64 |
| | | 12 ± 5 | 236 ± 45 | 18 ± 9 | 24 ± 7 | 385 ± 51 | 1089 ± 155 | | | |
| DBA/1 | mDCs | 64 ± 10/ | 63 ± 8/ | 76 ± 12/ | 72 ± 9/ | 81 ± 13/ | 74 ± 11/ | 33541 ± 2154 | 11815 ± 1554 | 3451 ± 364 |
| mice | | 198 ± 33 | 302 ± 48 | 355 ± 42 | 945 ± 54 | 611 ± 58 | 2159 ± 266 | | | |
| (H-2^{q}) | | | | | | | | | | |
| (n = 10) | rDCs | 3 ± 2/ | 51 ± 9/ | 5 ± 3/ | 7 ± 2/ | 46 ± 7/ | 71 ± 14/ | 297 ± 14 | 133 ± 14 | 128 ± 34 |
| | | 10 ± 4 | 244 ± 32 | 19 ± 8 | 21 ± 6 | 344 ± 53 | 1124 ± 265 | | | |
| BALB/c | rDCs^{b} | 5 ± 3/ | 57 ± 7/ | 6 ± 2/ | 8 ± 4/ | 52 ± 12/ | 70 ± 8/ | 425 ± 84 | 189 ± 72 | 121 ± 34 |
| mice | | 18 ± 5 | 231 ± 29 | 30 ± 9 | 42 ± 12 | 378 ± 51 | 1243 ± 194 | | | |
| (H-2^{d}) | | | | | | | | | | |
| (n = 10) | | | | | | | | | | |

^{a}The value of (³H)thymidine incorporation of T cells alone was less than 100 cpm.
^{b}rDCs were obtained from the spleen in rDC (H-2^{d})-treated recipients (H-2^{b}) of allogeneic BMS (H-2^{q}) on 5 days after transplantation as described in Experimental Procedures.

Lymphocytes having different histocompatible antigens (antigen-presenting cells and T cells) were subjected to mixed-culture. Thus, the capacity of T cells to activate and to grow alloantigens can be inspected in vitro. The capacities of murine immunoregulatory DCs and murine normal mature DCs to activate allogeneic T cells were inspected in the following manner. More specifically, unprimed or primed CD4⁺ T cells (2×10⁵ cells) and X-ray (15 Gy)-irradiated T cells were cultured together with allogeneic murine normal mature DCs, murine immunoregulatory DCs, or dihydroxyvitamin D₃-stimulated DCs (10³ to 2×10⁵ cells) in the presence or absence of recombinant murine IL-2 at 37° C. in 5% CO₂ in a 96-well culture plate for 3 days. Thereafter, ³H thymidine (Amersham Life Science, Buchinghamshire, UK) was added in an amount of 1 μCi/well, and culture was further conducted at 37° C. in 5% CO₂ in a 96-well culture plate for 16 hours. ³H thymidine that had been incorporated in cells was recovered onto a glass filter from the culture plate using a cell harvester, dehydrated, thoroughly penetrated with aquasol, and packaged in a dedicated-purpose film. The β-dose was measured using a β counter to inspect the activation of T cell growth caused by DCs. This demonstrates that murine normal mature DCs (H-2^{d}) potently activate allogeneic CD4⁺ T cells (H-2^{b} or H-2^{k}). In contrast, the capacity of murine immunoregulatory DCs to activate allogeneic CD4⁺ T cells was lower than that of murine normal mature DCs dihydroxyvitamin D₃-stimulated DCs known as immunotolerance-inducible DCs (Table 1 and FIG. 7B). Capacities of murine immunoregulatory DCs derived from all the examined mouse strains (H-2^{d}, H-₂^{b}, and H-₂^{q}) to activate allogeneic T cells tended to be similar to one another (Table 1).

Influence of murine immunoregulatory DCs upon CD4⁺ T cells subjected to allogeneic stimulation in vivo was inspected. Specifically, CD4⁺ T cells (I-K^{b+} CD4⁺) obtained from recipient mice subjected to allogeneic bone marrow transplantation were cultured with mature normal DCs derived from allogeneic mice or murine immunoregulatory DCs (H-2^{d}) in a plastic culture plate in accordance with the method described in Example 6, and activation of CD4⁺ T cells was inspected. When murine normal mature DCs (H-2^{d}) were further added to a culture system of I-K^{b+} CD4⁺ T cells in combination with murine normal mature DCs (H-2^{d}), the activation of I-K^{b+} CD4⁺ T cells was slightly enhanced. In contrast, when murine immunoregulatory DCs (H-2^{d}) were added to the same culture system, activation induced by murine normal mature DCs (H-2^{d}) was suppressed in accordance with the number of murine immunoregulatory DCs added. In contrast, when murine normal mature DCs prepared from mice of the same strain as I-K^{b+} CD4⁺ T cells (H-2^{b}) or an unrelated mouse strain (H-2^{q}) were added, I-K^{b+} CD4⁺ T cells were activated in a synergistic and more potent manner. When murine immunoregulatory DCs derived from an unrelated mouse strain (H-2^{q}) were added, no or substantially no influence was imposed upon the activation of I-K^{b}+ CD4⁺ T cells caused by murine normal mature DCs (H-2^{b}) (FIG. 7C). Accordingly, suppression of CD4⁺ T cell activation induced by murine immunoregulatory DCs was suggested to be an antigen-specific response. Similar experimental results were obtained with DC-T cell combinations of a strain different from the aforementioned one (FIG. 7D).

Influence of murine immunoregulatory DCs on the activity of CD8⁺ T cells subjected to allogeneic stimulation in vivo was inspected. Specifically, CD8⁺ T cells (I-K^{b+} CD8⁺) obtained from recipient mice subjected to allogeneic bone marrow transplantation were cultured with mature normal DCs derived from allogeneic mice or murine immunoregulatory DCs (H-2^{d}) in a plastic culture plate in accordance with the method described in Example 6, and the activation of CD8⁺ T cells was inspected. Cytotoxicity of CD8⁺ T cells stimulated in vivo was inspected in the following manner. Specifically, CD8⁺ T cells stimulated in vivo and in vitro were mixed with P815 cells, EL4 cells, and Con A-blast cells that had been radioactively labeled with Na⁵¹CrO₄ (10⁴ cells) at various mixing ratios, the resultants were subjected to culture for 4 hours, the culture supernatants were recovered, and the activity of radioactive substances contained therein was assayed. I-K^{b+} CD8⁺ T cells subjected to allogeneic stimulation in vivo exhibited potent cytotoxicity against the cell strain P815 (H-2^{d}), which was syngeneic to the stimulation. However, they did not exhibit any activity against the cell strain EL4 (H-2^{b}) or Con A-blast (H-2^{q}), which were strains different therefrom (FIG. 7E). This indicates that cytotoxicity induced in CD8⁺ T cells stimulated with H-2^{d} in vivo is specific to H-2^{d}. When I-K^{b+} CD8⁺ T cells subjected to stimulation in vivo were cultured together with murine immunoregulatory DCs (H-2^{d}), however, their cytotoxicity against P815 was significantly suppressed. When they were cultured together with syngeneic murine immunoregulatory DCs (H-2^{b}) or the immunoregulatory DCs of unrelated mice (H-2^{q}), their activity was not substantially affected (FIG. 7E). These results indicate that suppression of CTL activity by murine immunoregulatory DCs was an antigen-specific response. Similar results were attained with combinations of different mouse strains (FIG. 7F).

The capacity of murine immunoregulatory DCs to induce immunotolerance to allogeneic CD4⁺ T cells was inspected.

Allogeneic CD4⁺ T cells (H-2^{b}) that had been stimulated with murine normal mature DCs (H-2^{d}) in the primary culture strongly responded to secondary stimulation with murine normal mature DCs (H-2^{d}). Allogeneic CD4⁺ T cells (H-2^{b}) that had been stimulated with murine immunoregulatory DCs (H-2^{d}) exhibited low reactivity with the secondary stimulation with murine normal mature DCs (H-2^{d}). Growth of CD4⁺ T cells, however, was restored with the addition of IL-2 when intensive stimulation took place. In contrast, CD4⁺ T cells (H-2^{b}) that had been stimulated with murine normal mature DCs (H-2^{d}) exhibited reactivity equivalent to the response of unprimed CD4⁺ T cells (H-2^{b}) against secondary stimulation with mature normal DCs (H-2^{q} or H-2^{k}) derived from the unrelated mice. CD4⁺ T cells that had been stimulated with murine immunoregulatory DCs (H-2^{d}) exhibited slightly weaker reactivity with mature normal DCs (H-2^{q} or H-2^{k}) derived from the unrelated mice (FIG. 7G). Similar results were attained with combinations of different mouse strains (FIG. 7H).

### EXAMPLE 9

### Therapeutic Effects of Murine Immunoregulatory DCs on Acute GvHD After Allogeneic Transplantation

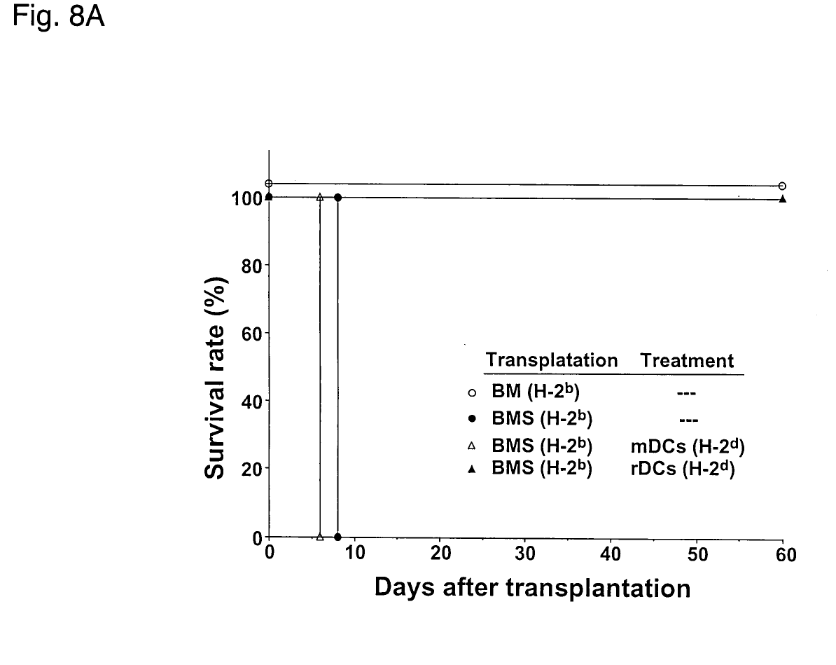

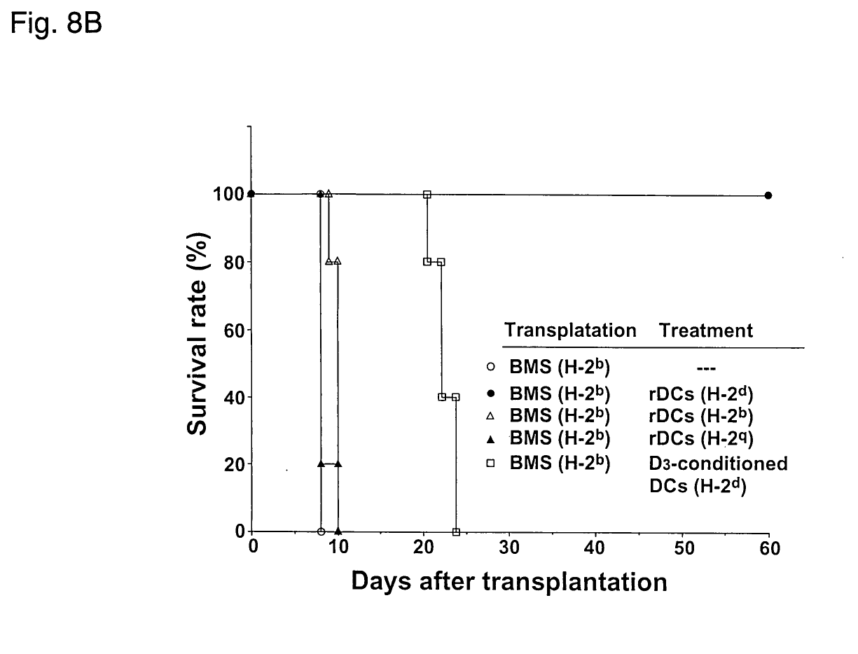

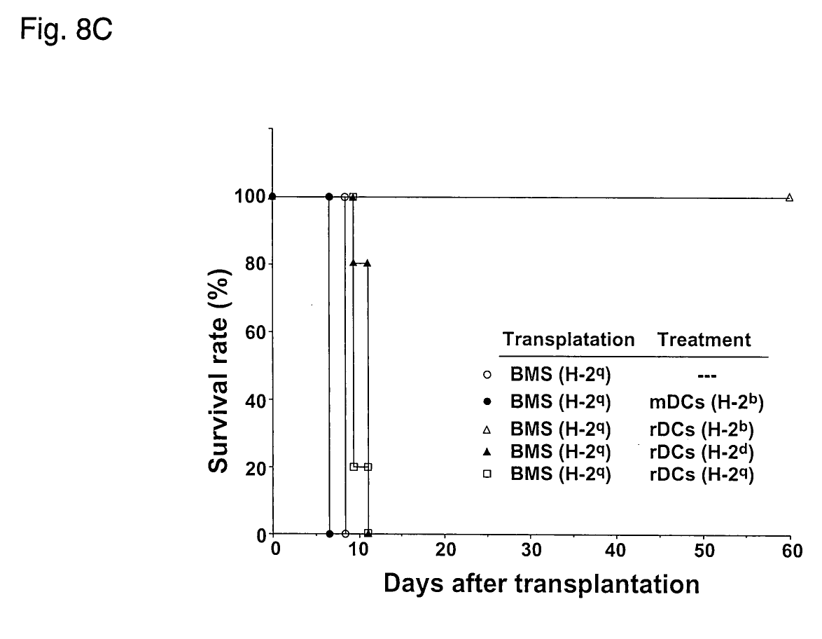

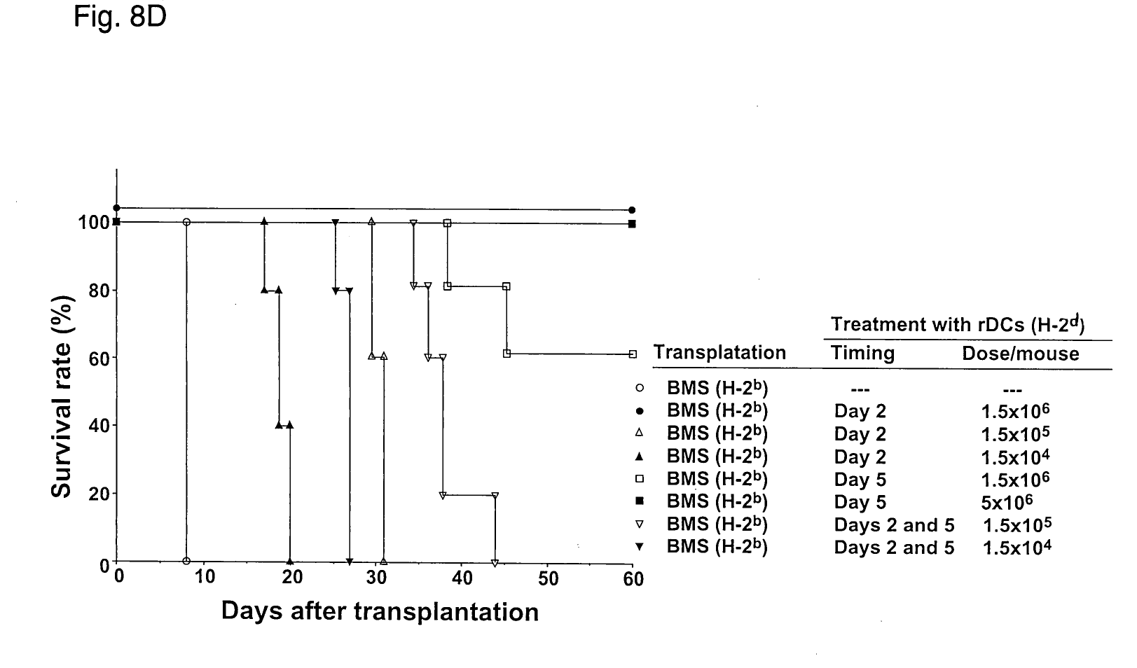

In order to analyze the therapeutic effects of murine immunoregulatory DCs on acute GvHD caused after allogeneic bone marrow transplantation, the influence of murine immunoregulatory DCs on lethal acute GvHD, which was developed in the recipient mice to which allogeneic bone marrow cells and spleen mononuclear cells had been transplanted, was inspected in the following manner. PBS (0.2 ml) consisting of bone marrow cells derived from allogeneic donor mice (1.5×10⁷ cells/mouse) or PBS (0.4 ml) comprising the aforementioned bone marrow cells and spleen mononuclear cells (1.5×10⁷ cells/mouse) were transplanted via injection into caudal veins of recipient mice (H-2^{d} or H-2^{b}, each group consisting of 5 individuals) irradiated with lethal doses of X-rays (10 Gy/mouse). Two or five days after transplantation, murine normal mature DCs derived from the syngeneic or allogeneic strains of the recipient mice, murine immunoregulatory DCs, or dihydroxyvitamin D₃-stimulated DCs were administered to the recipient mice in amounts of 1.5×10⁴ to 5.0×10⁶ cells/0.2 ml/mouse once or twice. The aforementioned recipient mice into which allogeneic bone marrow cells and spleen mononuclear cells had been transplanted were subjected to observation once a day until they died of GvHD or until 60 days had passed after the transplantation, in order to inspect their survival periods and changes in body weights. Allogeneic bone marrow cell- or spleen mononuclear cell (H-2^{b})-transplanted recipient mice (H-2^{d}) developed significant symptoms of acute GvHD such as piloerection, lowered motility, and decreased body weights within 6 days after transplantation, and all individuals died within 8 days after transplantation. Individuals in the group to which murine normal mature DCs (H-2^{d}) had been administered once in amounts of 1.5×10⁶ cells/mouse 2 days after bone marrow transplantation died before acute GvHD was developed. However, recipient mice of the group to which murine immunoregulatory DCs (H-2^{d}) of their syngeneic mice had been administered once 2 days after bone marrow transplantation in amounts of 1.5×10⁶ cells/mouse did not die, and survived until 60 days after transplantation. In this case, no or substantially no symptoms of acute GvHD were observed (FIG. 8A). Murine immunoregulatory DCs exhibited more potent therapeutic effects on acute GvHD than dihydroxyvitamin D₃-stimulated DCs (FIG. 8B). Similar results were attained with combinations of different mouse strains (FIG. 8C).

Single administration of a different number of murine immunoregulatory DCs, i.e., 1.5×10⁴ cells/mouse, 1.5×10⁵ cells/mouse, or 1.5×10⁶ cells/mouse, was carried out 2 days after bone marrow transplantation, and the therapeutic effects on acute GvHD were found to vary in a dose-dependent manner (FIG. 8D). Also, when murine immunoregulatory DCs (1.5×10⁴ cells/mouse or 1.5×10⁵ cells/mouse) were administered 2 days after transplantation or 2 days and 5 days after transplantation and when murine immunoregulatory DCs (1.5×10^{6′} cells/mouse) were administered 5 days after transplantation, the survival of recipient mice was prolonged (FIG. 8D). In contrast, a single administration of murine immunoregulatory DCs (1.5×10⁶ cells/mouse) 5 days after bone marrow transplantation significantly lowered the therapeutic effects. A single administration of murine immunoregulatory DCs (5.0×10⁶ cells/mouse) allowed all recipient mice to survive (FIG. 8D).

### EXAMPLE 10

### Influence of the Administered Murine Immunoregulatory DCs on Immune Responses of Allogeneic Bone-Marrow-Transplanted Recipients and the Half-Lives Thereof

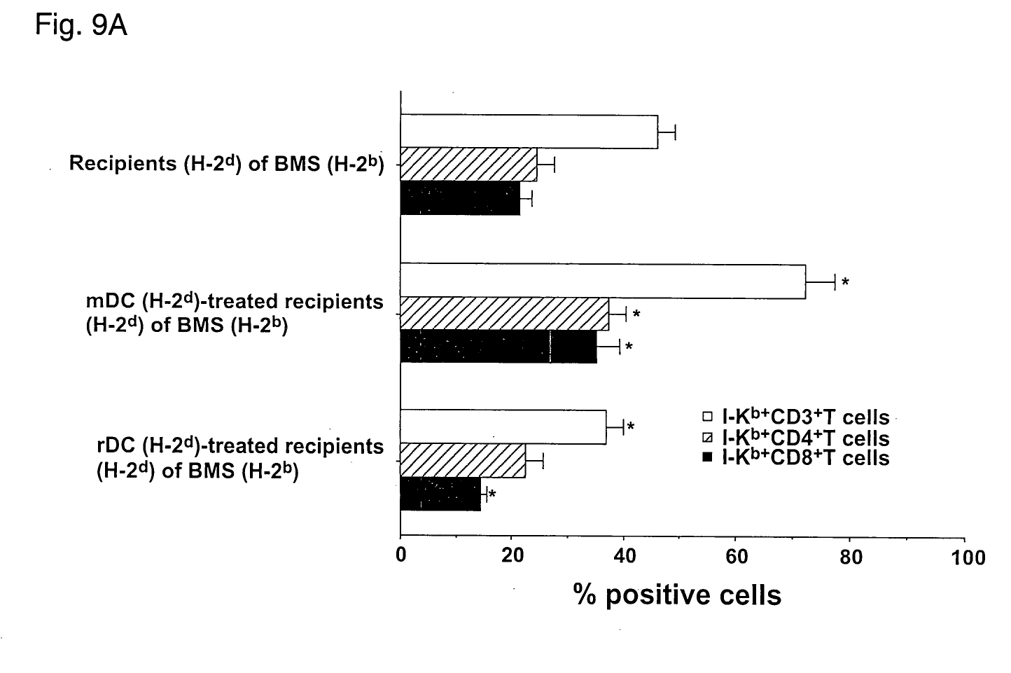

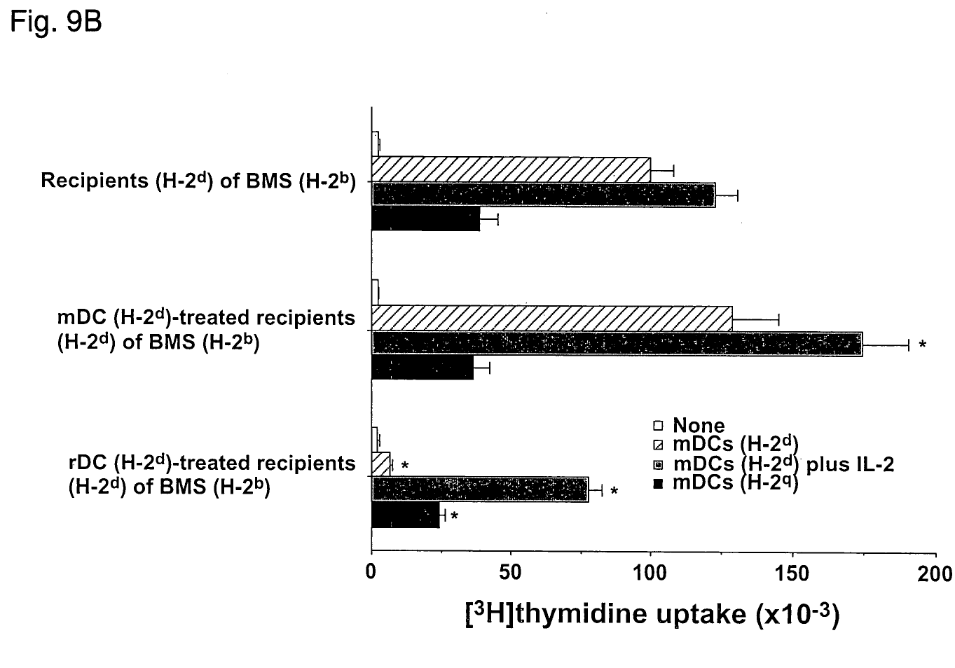

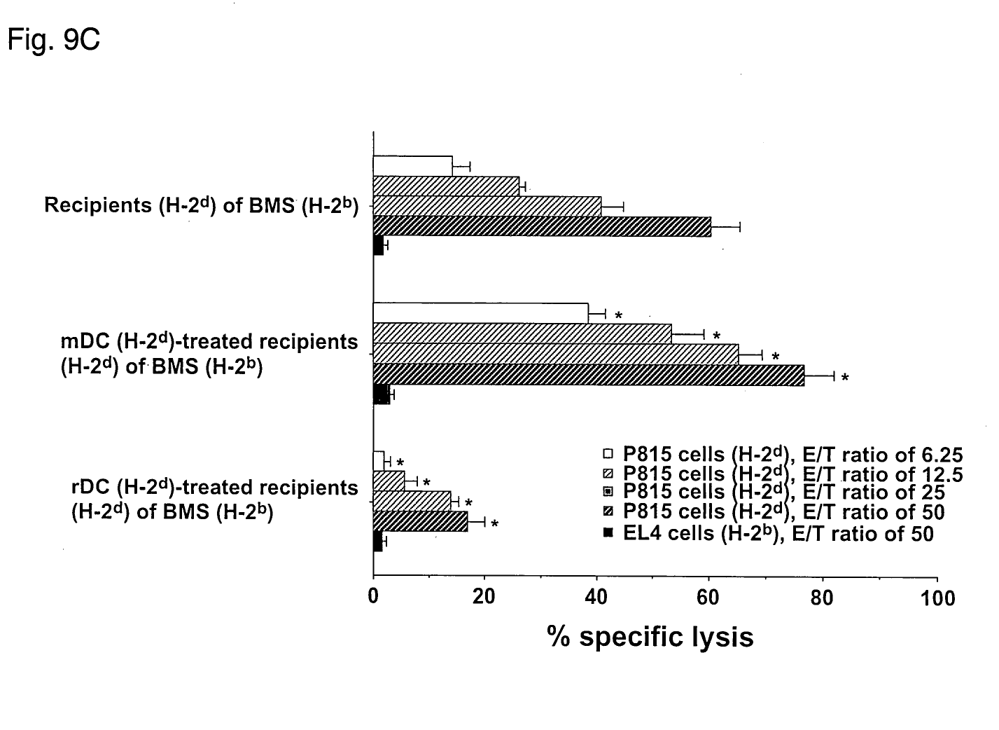

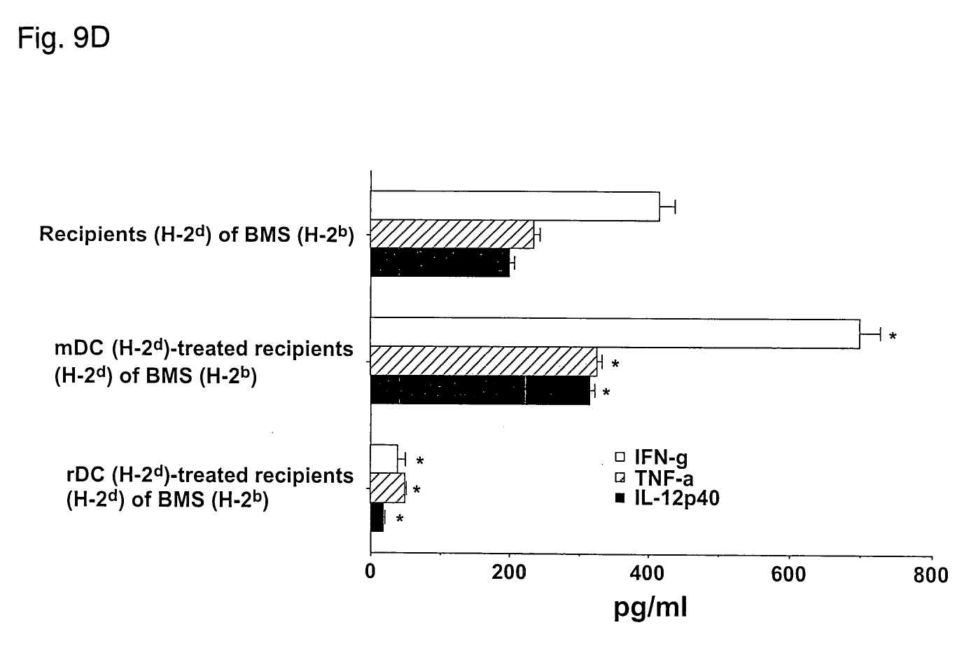

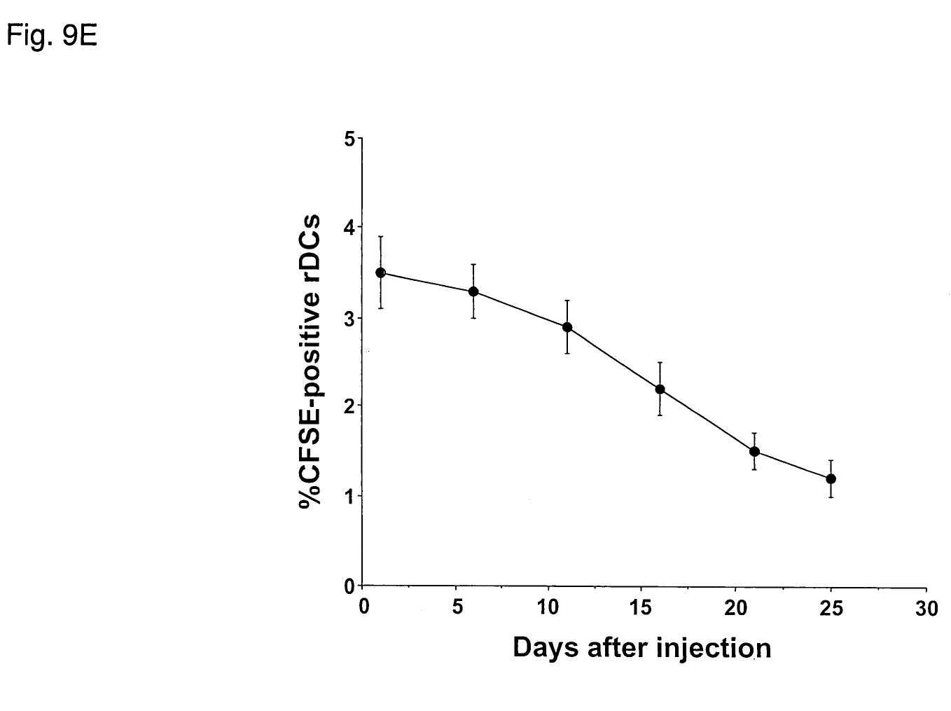

Donor-derived I-K^{b+} T cells in the spleen mononuclear cells of the recipient mice 5 days after bone marrow transplantation were analyzed. The contents of I-K^{b+} CD3⁺ T cells, I-K^{b+} CD4⁺ T cells, and I-K^{b+} CD8⁺ T cells in the spleen mononuclear cells of the recipient mice to which murine normal mature DCs had been administered after bone marrow transplantation were significantly increased compared with those in the recipient mice to which DCs had not been administered. In contrast, I-K^{b +} CD3⁺ T cells and I-K^{b +} CD8⁺ T cells in the recipient mice to which murine immunoregulatory DCs had been administered significantly decreased compared with those in the recipient mice to which DCs had not been administered. Although there was no significant difference with regard to the I-K^{b +} CD4⁺ T cell contents, they were significantly decreased (FIG. 9A).

Allogeneic reactivity of I-K^{b +} CD4⁺ T cells against murine normal mature DCs in the recipient mice to which bone marrow had been transplanted was inspected. I-K^{b +} CD4⁺ T cells prepared from recipient mice to which DC had not been administered or murine normal mature DCs had been administered strongly responded to murine normal mature DCs (H-2^{d}). In contrast, I-K^{b +} CD4⁺ T cells prepared from recipient mice to which murine immunoregulatory DCs had been transplanted exhibited low reactivity with murine normal mature DCs (H-2^{d}), and this reactivity was restored with the addition of recombinant murine IL-2. The reactivity of I-K^{b+} CD4⁺ T cells with the mature normal DCs of unrelated mice (H-2^{q}) was lower than that of (H-2^{d}) in any of the treated recipients (FIG. 9B).

For the purpose of inspecting the cytotoxicity of donor-derived I-K⁺ CD8⁺ T cells against the recipients' tissues (H-2^{d}) in the bone marrow-transplant recipients, the cytotoxicity of I-K^{b +} CD8⁺ T cells prepared from the recipients against P815 and EL4 was inspected. I-K^{b +} CD8⁺ T cells derived from the recipients to which murine normal mature DCs had been administered exhibited higher cytotoxicity against P815 than that derived from the recipients to which DCs had not been administered. In contrast, the cytotoxicity against P815 of I-K^{b +} CD8⁺ T cells derived from the recipients to which murine immunoregulatory DCs had been administered was significantly low. In these I-K^{b +} CD8⁺ T cells, no or substantially no cytotoxicity against EL4 was observed. It was suggested that the cytotoxicity of I-K^{b+} CD8⁺ T cells was specific to H-2^{d} (FIG. 9C).

The inflammatory cytokine content in the serum of the recipients 5 days after bone marrow transplantation was inspected. The content of IFN-γ, TNF-α, and IL-12 p40 in the serum of the recipients to which murine normal mature DCs had been administered was significantly higher than that in the recipients to which DCs had not been administered. In contrast, the content of IFN-γ, TNF-α, and IL-12 p40 in the serum of the recipients to which murine immunoregulatory DCs had been administered was significantly lower than that in the recipients to which DCs had not been administered (FIG. 9D).

For the purpose of inspecting the half-lives of murine immunoregulatory DCs that had been administered to the recipient mice, murine immunoregulatory DCs (H-2^{d}) to which carboxyfluorescein diacetate-succinimidyl estate (CFSE) had been added was administered to the bone marrow-transplanted recipients. Migration thereof to the spleen was inspected using a flow cytometer. In the spleen mononuclear cells 1 day after administration of DCs, about 4% thereof were found to be CFSE⁺ murine immunoregulatory DCs, and the half-life of the murine immunoregulatory DCs administered was approximately 18 days after administration (FIG. 9E). In order to inspect the stability of murine immunoregulatory DCs under inflammation-inducing conditions after transplantation in an organism, the expression of cell surface molecules and capacity for activating allogeneic T cells of murine immunoregulatory DCs prepared from the recipient mice (H-2^{b}) to which bone marrow (H-2^{q}) had been transplanted and murine immunoregulatory DCs (H-2^{d}) had been administered 5 days after transplantation were inspected (Table 1). This indicated that there were no or substantially no changes in properties of murine immunoregulatory DCs due to transplantation in an organism, and properties of murine immunoregulatory DCs were maintained even under inflammation-inducing conditions in an organism.

### EXAMPLE 11

### Examination of the Involvement of Immunoregulatory T Cell Induction in Therapeutic Effects of the Administered Murine Immunoregulatory DCs in Allogeneic GvHD

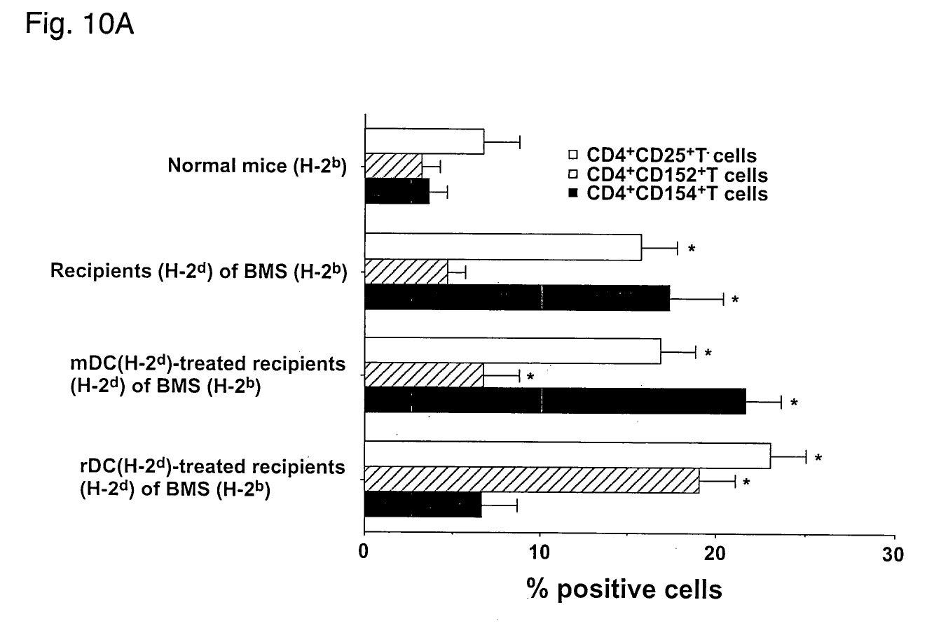

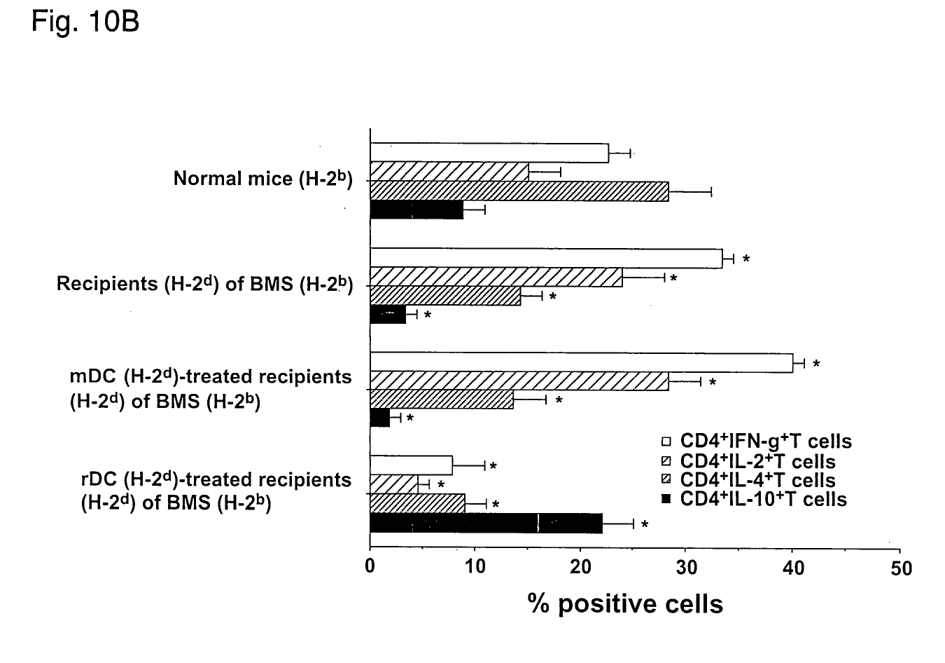

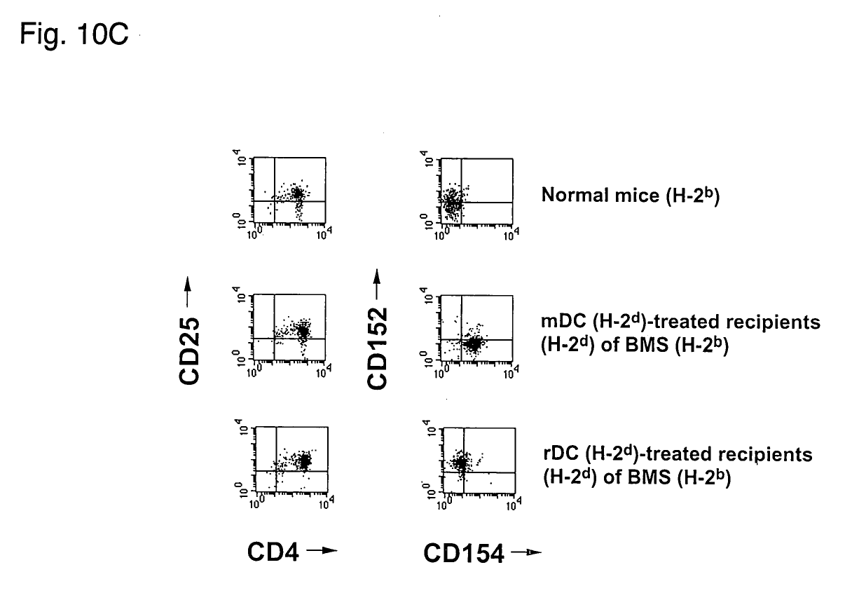

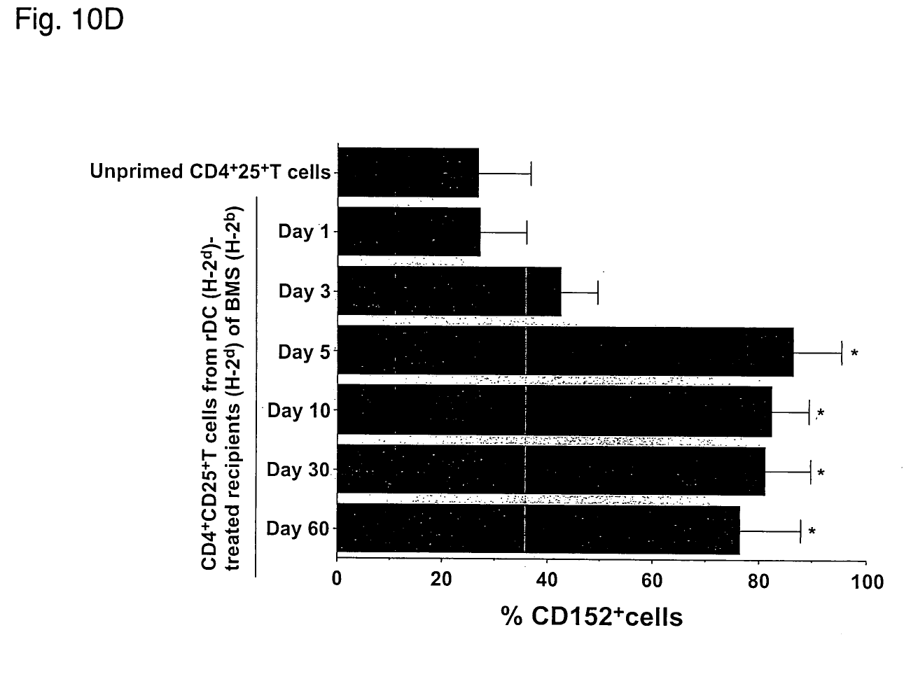

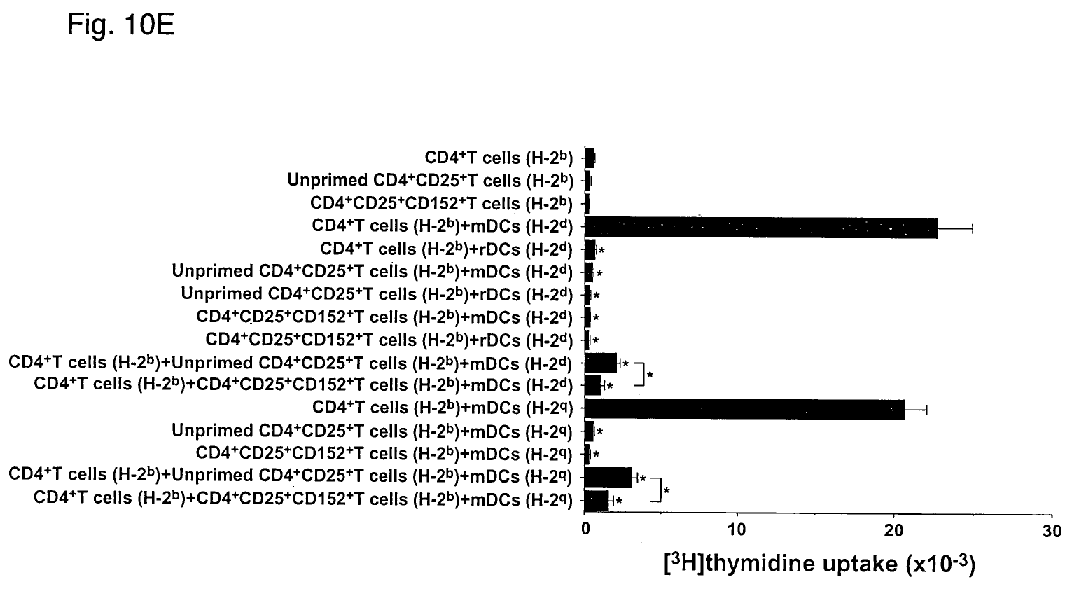

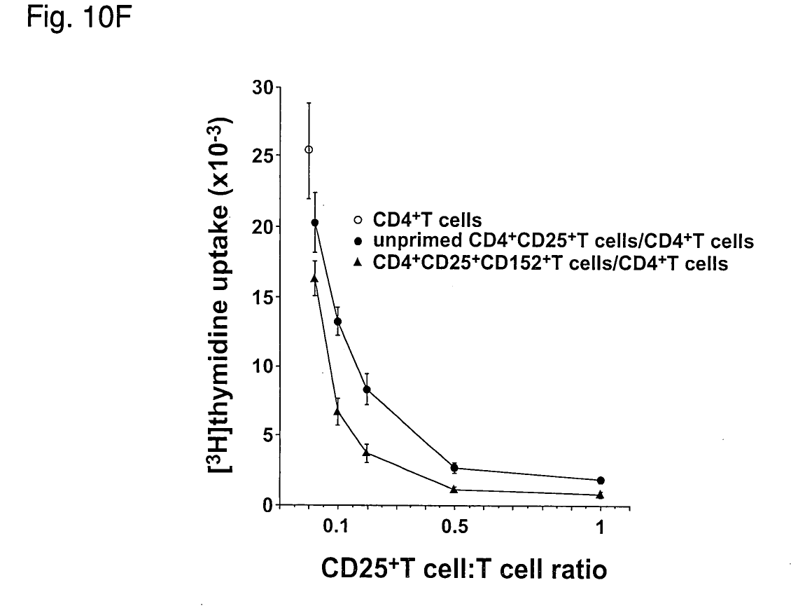

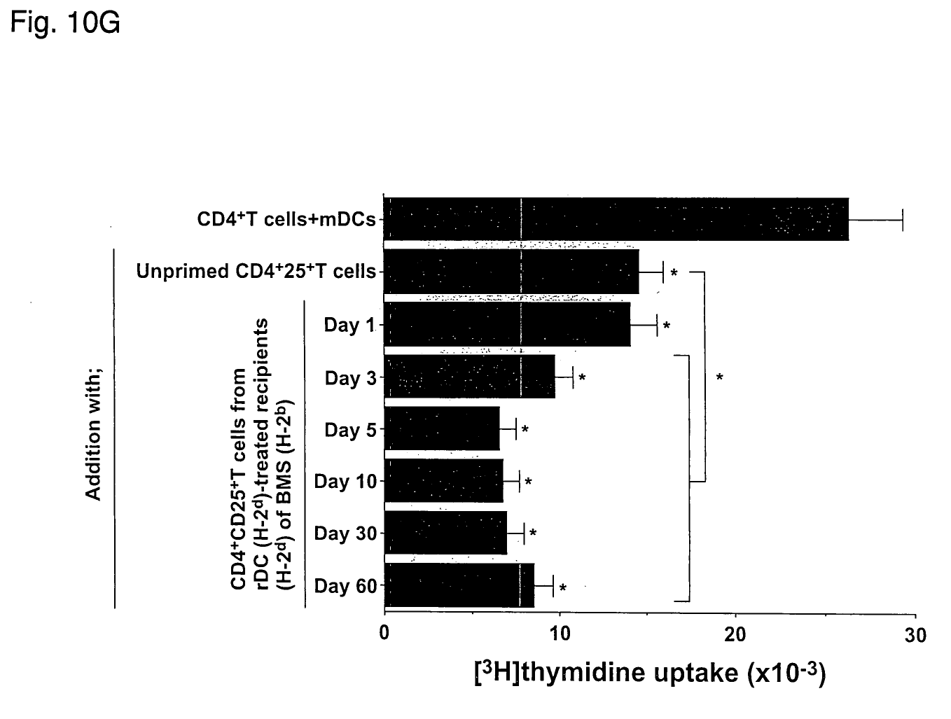

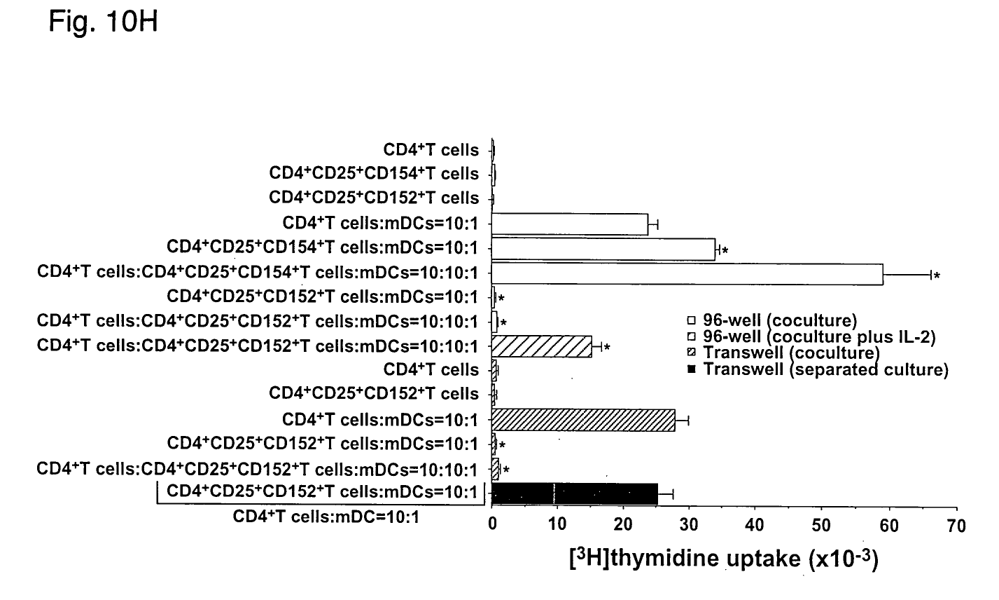

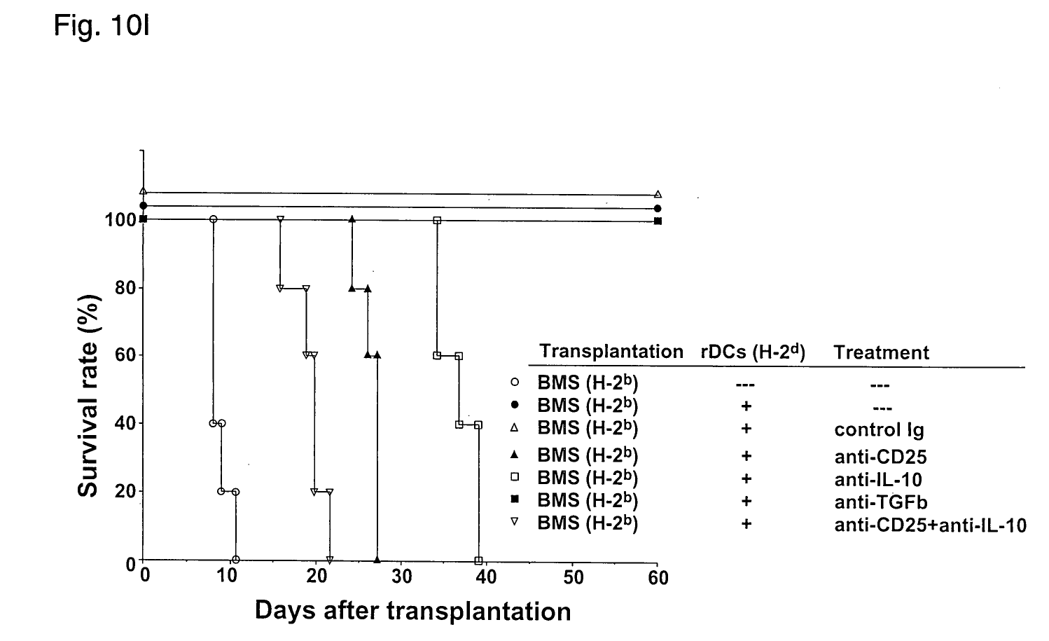

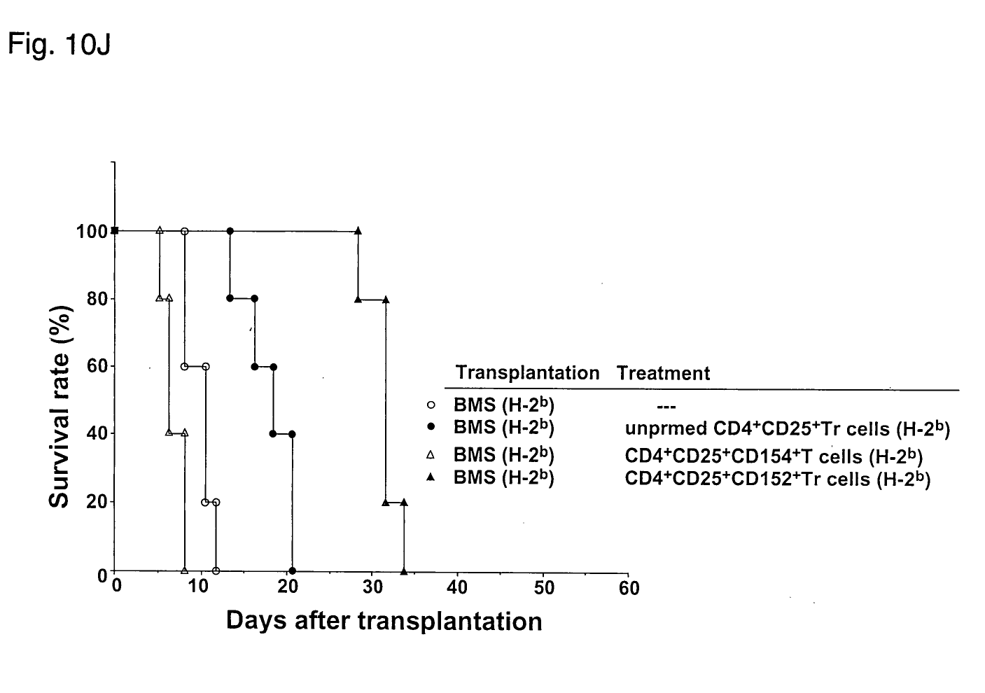

Donor-derived CD4⁺ T cells (H-2^{b}) were prepared from spleens of mice (H-2^{d}) after the transplantation of allogeneic bone marrow cells and spleen mononuclear cells (H-2^{b}) or spleens of mice (H-2^{d}) to which a variety of DCs (H-2^{b}) had been administered after the aforementioned transplantation 5 days after the transplantation. The ratios of CD25, CD152, and CD154 to be expressed were analyzed using FACS, and the results were compared with those of normal mice (H-2^{b}) without transplantation (FIG. 10A). Transplantation, administration of DCs, and preparation of donor-derived CD4⁺ T cells were carried out in accordance with the method described in Example 6. In the group to which only allogeneic bone marrow cells and spleen mononuclear cells had been transplanted (recipients (H-2^{d}) of BMS (H-2^{b})) and in the group to which murine normal mature DCs had been administered subsequent to the transplantation (mDC (H-2^{d})-treated recipients (H-2^{d}) of BMS (H-2^{b})), the ratios of CD25 and CD154 to be expressed were higher than those in the case of normal mice. In contrast, the ratios of CD25 and CD152 to be expressed were higher in the group to which allogeneic bone marrow cells and spleen mononuclear cells had been transplanted and murine immunoregulatory DCs had been then administered (rDC (H-2^{d})-treated recipients (H-2^{d}) of BMS (H-2^{b})).

As described above, donor-derived CD4⁺ T cells (H-2^{b}) were prepared from spleens of mice (H-2^{d}) after the transplantation of allogeneic bone marrow cells and spleen mononuclear cells (H-2^{b}) or spleens of mice (H-2^{d}) to which a variety of DCs (H-2^{b}) had been administered after the aforementioned transplantation 5 days after the transplantation. Intracellular cytokines after the secondary stimulation carried out in the aforementioned manner were analyzed using FACS (FIG. 10B). Transplantation, administration of DCs, preparation of donor-derived CD4⁺ T cells, and analysis of intracellular cytokines were carried out in the manner described above. In the group to which only allogeneic bone marrow cells and spleen mononuclear cells had been transplanted (recipients (H-2^{d}) of BMS (H-2^{b})) and in the group to which murine normal mature DCs had been administered subsequent to the transplantation (mDC (H-2^{d})-treated recipients (H-2^{d}) of BMS (H-2^{b})), the IFN-γ-producing cell content and IL-2-producing cell content increased compared with those in normal mice (H-2^{b}). In contrast, the IL-10-producing cell content increased in the group to which allogeneic bone marrow cells and spleen mononuclear cells had been transplanted and murine immunoregulatory DCs had been then administered (rDC (H-2^{d})-treated recipients (H-2^{d}) of BMS (H-2^{b})).

Donor-derived CD4⁺ CD25⁺ T cells (H-2^{b}) from the spleen mononuclear cells of mice (H-2^{d}) to which a variety of DCs (H-2^{d}) had been administered after transplantation of allogeneic bone marrow cells and spleen mononuclear cells (H-2^{b}) were obtained, and CD152 and CD154 expression thereof was compared with those in CD4⁺ CD25⁺ T cells of normal mice (FIG. 10C). Transplantation and administration of DCs were carried out in the manner described above. CD4⁺ CD25⁺ T cells of normal mice were prepared from CD4⁺ T cells of the spleen cells obtained in the manner described above using an anti-CD25 antibody (Clone PC61, BD PharMingen) and a magnetic-beads-coupled anti-rat IgG sheep antibody (Dynal). Donor-derived CD4⁺ CD25⁺ T cells of mice that had undergone transplantation and administration of a variety of DCs were similarly prepared from the donor-derived CD4⁺ T cells obtained in the manner described above using an anti-CD25 antibody and a magnetic-beads-coupled anti-rat IgG sheep antibody. Purity of the prepared CD4⁺ CD25⁺ cells was found to be 90% or higher as a result of analysis using FACS. CD154 and CD152 expression of the thus obtained cells were analyzed using FACS. In unprimed CD4⁺ CD25⁺ T cells obtained from normal mice (H-2^{b}), CD152 was constitutively expressed in some cells, although expression of CD154 was not observed as reported in the past (Takahashi et al., 2000, J. Exp. Med. 192, 303-309). In contrast, CD154 was expressed in most of donor-derived CD4⁺ CD25⁺ T cells of mice to which allogeneic bone marrow cells and spleen mononuclear cells had been transplanted and murine normal mature DCs had been then administered (mDC (H-2^{d})-treated recipients (H-2^{d}) of BMS (H-2^{b})). In most of the donor-derived CD4⁺ CD25⁺ T cells of mice to which allogeneic bone marrow cells and spleen mononuclear cells had been transplanted and murine immunoregulatory DCs had been then administered (rDC (H-2^{d})-treated recipients (H-2^{d}) of BMS (H-2^{b})), CD152 was expressed.

Donor-derived CD4⁺ CD25⁺ T cells (H-2^{b}) in the spleens were prepared from mice (H-2^{d}) to which allogeneic bone marrow cells and spleen mononuclear cells (H-2^{b}) had been transplanted and murine immunoregulatory DCs (H-2^{d}) had been then administered (2 days after transplantation), and changes in the ratios of CD152 to be expressed with the elapse of time were analyzed using FACS on 1, 3, 5, 10, 30, and 60 days after transplantation (FIG. 10D). Transplantation, administration of DCs, and preparation of donor-derived CD4⁺ CD25⁺ T cells were carried out in the manner described above. Compared with unprimed CD4⁺ CD25⁺ T cells of normal mice (H-2^{b}), the ratio of CD152 to be expressed was elevated after administration of murine immunoregulatory DCs and the high positive ratio was maintained until 60 days after administration in the CD4⁺ CD25⁺ T cells derived from mice to which allogeneic bone marrow cells and spleen mononuclear cells had been transplanted and murine immunoregulatory DCs had been then administered (rDC (H-2^{d})-treated recipients (H-2^{d}) of BMS (H-2^{b})).

Reactivity of CD4⁺ T cells (H-2^{b}) stimulated with mature murine allogeneic DCs (mDCs H-2^{d}), that of unprimed CD4⁺ CD25⁺ T cells (H-2^{b}), and that of donor-derived CD4⁺ CD25⁺ CD152⁺ T cells (H-2^{b}) of mice (H-2^{d}) to which allogeneic bone marrow cells and spleen mononuclear cells (H-2^{b}) had been transplanted and murine immunoregulatory DCs (H-2^{d}) had been transplanted were compared in the same manner as in Example 8 (FIG. 10E). The ratio of the number of T cells to that of mature murine allogeneic DCs was 10:1. Donor-derived CD4⁺ CD25⁺ T cells of mice to which allogeneic bone marrow cells and spleen mononuclear cells had been transplanted and immunoregulatory DCs had been then administered exhibited low response to mature murine allogeneic DC stimulation, as with the case of unprimed CD4⁺ CD25⁺ T cells. None of the cells responded to murine allogeneic immunoregulatory DCs stimulation (rDCs (H-2^{d})). A variety of CD4⁺ CD25⁺ T cells (H-2^{b}) were added to a mixed-culture system of CD4⁺ T cells (H-2^{b}) at the time of the aforementioned mature murine allogeneic DC stimulation (H-2^{d}) in amounts consisting of the same number of cells as CD4⁺ T cells so as to examine suppressing activity of CD4⁺ CD25⁺ T cells, and evaluation was carried out based on ³H thymidine incorporation on the third day of culture. Donor-derived CD4⁺ CD25⁺ T cells of mice to which allogeneic bone marrow cells and spleen mononuclear cells had been transplanted and immunoregulatory DCs had been then administered exhibited activity of suppressing the growth of CD4⁺ T cells, as with the case of unprimed CD4⁺ CD25⁺ T cells, and this suppressing activity was more potent than that of unprimed CD4⁺ CD25⁺ T cells. When the haplotype (mDCs (H-2^{q})) was different from that of murine immunoregulatory DCs (rDCs (H-2^{d})) to which mature murine allogeneic DCs for stimulation had been administered, similar suppressing activity was observed. Thus, suppressing activity of CD4⁺ CD25⁺ T cells derived from mice to which allogeneic bone marrow cells and spleen mononuclear cells had been transplanted and murine immunoregulatory DCs had been then administered was found to be antigen-nonspecific, as with the case of unprimed CD4⁺ CD25⁺ T cells.

In order to more precisely examine the level of suppressing activity of CD4⁺ CD25⁺ T cells shown in FIG. 10E, the number of CD4⁺ CD25⁺ T cells (H-2^{b}) to be added to the mixed-culture system of mature murine allogeneic DCs (H-2^{b}) with CD4⁺ T cells (H-2^{b}) was varied (FIG. 10F). Transplantation and administration of DCs were carried out in the manner described above. Donor-derived CD4⁺ CD25⁺ T cells (H-2^{b}) of mice (H-2^{d}) to which allogeneic bone marrow cells and spleen mononuclear cells (H-2^{b}) had been transplanted and murine immunoregulatory DCs (H-2^{d}) had been then administered were prepared in the manner described above 5 days after the transplantation. A smaller number of donor-derived CD4⁺ CD25⁺ CD152⁺ T cells of mice to which allogeneic bone marrow cells and spleen mononuclear cells had been transplanted and immunoregulatory DCs had been then administered than unprimed CD4⁺ CD25⁺ T cells was sufficient to exhibit potent suppressing activity, and enhanced activity of immunoregulatory T cells was observed with the administration of murine immunoregulatory DCs.

The way that suppressing activity of donor-derived CD4⁺ CD25⁺ T cells was enhanced with the elapse of time, which was caused by administration of immunoregulatory DCs (H-2^{d}) after transplantation of allogeneic bone marrow cells and spleen mononuclear cells (H-2^{b}), was examined (FIG. 10G). Transplantation and administration of DCs were carried out in the manner described above. Donor-derived CD4⁺ CD25⁺ T cells (H-2^{b}) were prepared from mice (H-2^{d}) to which allogeneic bone marrow cells and spleen mononuclear cells (H-2^{b}) had been transplanted and murine immunoregulatory DCs (H-2^{d}) had been administered (2 days after transplantation) on 1, 3, 5, 10, 30, and 60 days after transplantation (rDC (H-2^{d})-treated recipients (H-2^{d}) of BMS (H-2^{b})) in the manner described above, and suppressing activity was examined in the same manner as with the case shown in FIG. 10E. The suppressing activity was equivalent to that of unprimed CD4⁺ CD25⁺ T cells on day 1, although suppressing activity was enhanced after the administration of immunoregulatory DCs, and high suppressing activity was maintained until 60 days after the transplantation.

Reactivity and suppressing activity of donor-derived CD4⁺ CD25⁺ T cells (H-2^{b}) of mice (H-2^{d}) to which allogeneic bone marrow cells and spleen mononuclear cells (H-2^{b}) had been transplanted and murine normal mature DCs (H-2^{d}) had been then administered were examined in the same manner as that shown in FIG. 10E (FIG. 10H). Unlike the donor-derived CD4⁺ CD25⁺ CD152⁺ T cells (H-2^{b}) of mice to which murine immunoregulatory DCs had been administered, the donor-derived CD4⁺ CD25⁺ CD154⁺ T cells (H-2^{b}) of mice to which murine normal mature DCs had been administered exhibited more potent growth responses with mature murine allogeneic DCs stimulation (mDCs (H-2^{d})) than the CD4⁺ T cells (H-2^{b}). No activity of suppressing CD4⁺ T cell growth when added to the mixed-culture system for mature murine allogeneic DCs and CD4⁺ T cells was observed. Subsequently, the following experiment was carried out in order to examine the properties of suppressing activity observed in the donor-derived CD4⁺ CD25⁺ T cells (H-2^{b}) of mice (H-2^{d}) to which allogeneic bone marrow cells and spleen mononuclear cells (H-2^{b}) had been transplanted and immunoregulatory DCs (H-2^{d}) had been then administered. At the outset, suppression assay similar to that shown in FIG. 10E was carried out in the presence of 100 U/ml of IL-2 to examine the influence of IL-2 on suppressing activity. In the presence of IL-2, suppressing activity of donor-derived CD4⁺ CD25⁺ T cells of mice to which allogeneic bone marrow cells and spleen mononuclear cells had been transplanted and murine immunoregulatory DCs had been then administered was partially attenuated. A transwell experiment was further carried out in order to examine the dependence of suppressing activity on cell contact in the following manner. Mature murine allogeneic DCs (H-2^{d}, 10⁵ cells/well), CD4⁺ T cells (H-2^{b}, 10⁶ cells/well), and donor-derived CD4⁺ CD25⁺ T cells (H-2^{b}, 10⁶ cells/well) of mice to which allogeneic bone marrow cells and spleen mononuclear cells had been transplanted and murine immunoregulatory DCs had been then administered were mixed in a 24-well plate (coculture). Alternatively, CD4⁺ T cells and mature murine allogeneic DCs were cultured separately from CD4⁺ CD25⁺ T cells and mature murine allogeneic DCs using a transwell for 4 days (separated culture). Normal mature murine DCs were then removed, T cells remaining thereafter were transferred to a 96-well plate in amounts of 10⁵ cells/well, and ³H thymidine incorporation 5 days after the initiation of culture was evaluated. When cell contact between CD4⁺ T cells and CD4⁺ CD25⁺ T cells was blocked with a transwell, suppressing activity disappeared. This demonstrated the dependence of suppressing activity on cell contact.

Roles of IL-10-producing CD4⁺ T cells and those of CD4⁺ CD25⁺ T cells in therapeutic and ameliorating effects of murine immunoregulatory DCs on acute GvHD developed after allogeneic bone marrow transplantation were inspected in the following manner (FIG. 10I). In accordance with Example 9, allogeneic bone marrow cells and spleen mononuclear cells (H-2^{b}) were transplanted to recipient mice (H-2^{d}), and murine immunoregulatory DCs (1.5×10⁶ cells/mouse) were administered thereto on the second day. Thereafter, an anti-CD25 antibody (Clone PC61, BD PharMingen), an anti-IL-10-neutralizing polyclonal antibody (model AB-417-NA, R&D Systems, Minneapolis, Minn.), an anti-TGF-β-neutralizing antibody (Clone 1D11, R&D Systems, Minneapolis, Minn.), or control rat IgG were administered intravenously to mice in amounts of 500 μg/mouse 3, 5, 7, 9, 10, 13, and 15 days after transplantation. Administration of the aforementioned anti-CD25 antibody resulted in disappearance of 98% or more of CD4⁺ CD25⁺ T cells in the spleens of the recipient mice to which murine immunoregulatory DCs had been administered 16 days after transplantation. As a result, the effects of murine immunoregulatory DCs for ameliorating acute GvHD were significantly deteriorated by the administration of an anti-CD25 antibody or anti-IL-10 antibody, although no influence was imposed upon the aforementioned effects by the administration of an anti-TGF-β antibody or control rat IgG. Simultaneous administration of an anti-CD25 antibody and an IL-10 antibody more potently suppressed the effects of murine immunoregulatory DCs to ameliorate acute GvHD (FIG. 10I). Effects of single administration of CD4⁺ CD25⁺ immunoregulatory T cells, CD4⁺ CD25⁺ CD154⁺ immunoregulatory T cells, or CD4⁺ CD25⁺ CD152⁺ immunoregulatory T cells to recipient mice to which allogeneic bone marrow cells had been transplanted were inspected. Allogeneic bone marrow cells and spleen mononuclear cells (H-2^{b}) were transplanted to recipient mice (H-2^{d}) in accordance with Example 9, and the unprimed CD4⁺ CD25⁺ immunoregulatory T cells, CD4⁺ CD25⁺ CD154⁺ immunoregulatory T cells, and CD4⁺ CD25⁺ CD152⁺ immunoregulatory T cells prepared on the second day (H-2^{b}) were administered intravenously thereto once. As a result, the CD4⁺ CD25⁺ CD152⁺ immunoregulatory T cells exhibited more potent suppressing effects on acute GvHD compared with the unprimed CD4⁺ CD25⁺ immunoregulatory T cells. In contrast, CD4⁺ CD25⁺ CD154⁺ immunoregulatory T cells were apt to significantly exacerbate the suppressing effects (FIG. 10J).

### EXAMPLE 12

### Analysis of Phenotypes of Cells Induced from Allogeneic CD4⁺ CD25⁻ T Cells In Vitro using Murine Immunoregulatory DCs

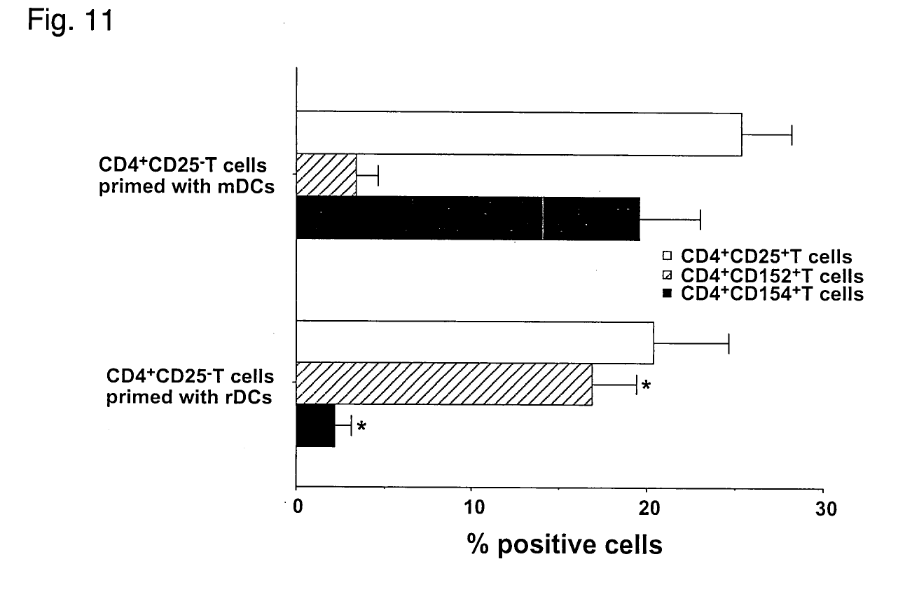

CD4⁺ mononuclear cells derived from spleens of C57BL/6 mice (H-2^{b}) were prepared in the manner described above, and CD25⁺ cells were removed therefrom using a rat anti-CD25 antibody and a magnetic-beads-coupled goat anti-rat IgG antibody. Thus, CD4⁺ CD25⁻ T cells with purity of 95% or higher were prepared. The resulting T cells were stimulated by being subjected to mixed culture with murine normal mature DCs or murine immunoregulatory DCs prepared from BALB/c mice (H-2^{d}) in a manner described above at a mixing ratio of 10:1 (T cells:DCs). Five days after the initiation of mixed culture, DC fractions were removed using a rat anti-I-K^{d} antibody and a magnetic-beads-coupled goat anti-rat IgG antibody to prepare T cell fractions, and the resultants were analyzed by flow cytometry. As shown in FIG. 11, the CD154⁺ cell content was high and the CD152⁺ cell content was low in T cells stimulated with murine normal mature DCs. In contrast, the CD154⁺ cell content was significantly lower and the CD152⁺ cell content was significantly higher in T cells stimulated with murine immunoregulatory DCs than in T cells stimulated with murine normal mature DCs. This indicates that murine immunoregulatory DCs can also induce CD152⁺ cells in vitro.

### EXAMPLE 13

### Influence of Murine Immunoregulatory DCs on Graft-Versus-Leukemia Effects in Tumor-Bearing Mice Transplanted with Bone Marrow Cells and Spleen Mononuclear Cells

P815 mastocytomas (2×10⁵ cells/0.2 ml, H-2^{d}, RIKEN Cell Bank, Tsukuba, Japan) were administered intravenously to BALB/c mice (H-2^{d}, each group consisting of 5 individuals). Two days thereafter, mice were systemically irradiated with lethal doses of radiation (10 Gy/mouse, source: ⁶⁰Co, MBR-1505R2, Hitachi Medical, Tokyo, Japan), and a group to which host-incompatible bone marrow nucleated cells (BM, 1.5×10⁷ cells suspended in 0.2 ml of phosphate buffer) prepared in a manner described above or host-incompatible bone marrow nucleated cells and spleen mononuclear cells (BMS, a mixture of 1.5×10⁷ cells each suspended in 0.4 ml of phosphate buffer) were administered via tail veins were prepared. The influence of murine immunoregulatory DCs (rDCs) on graft-versus-leukemia effects was observed by administering murine immunoregulatory DCs to the group to which host-incompatible bone marrow nucleated cells (BM) and spleen mononuclear cells (BMS) (a mixture of 1.5×10⁷ cells each suspended in 0.4 ml of phosphate buffer) had been administered on the second day.

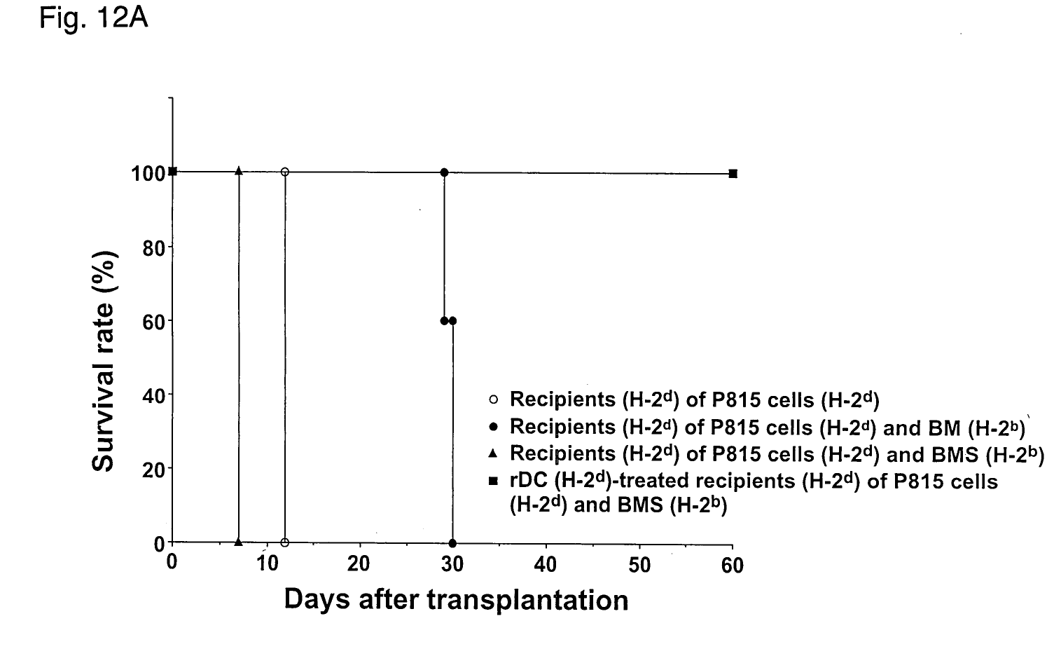

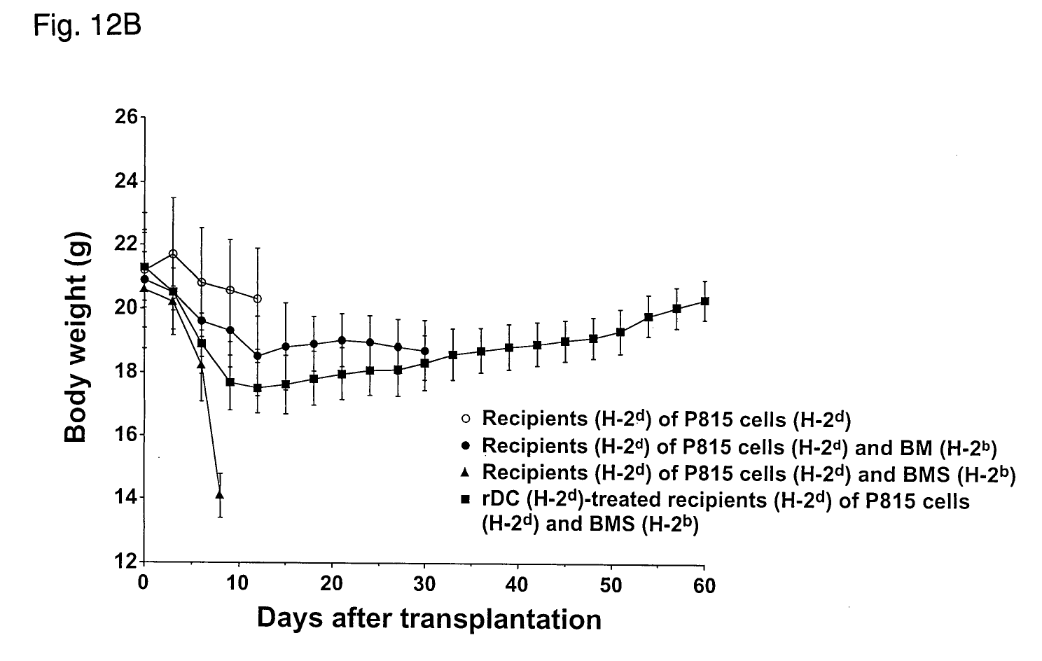

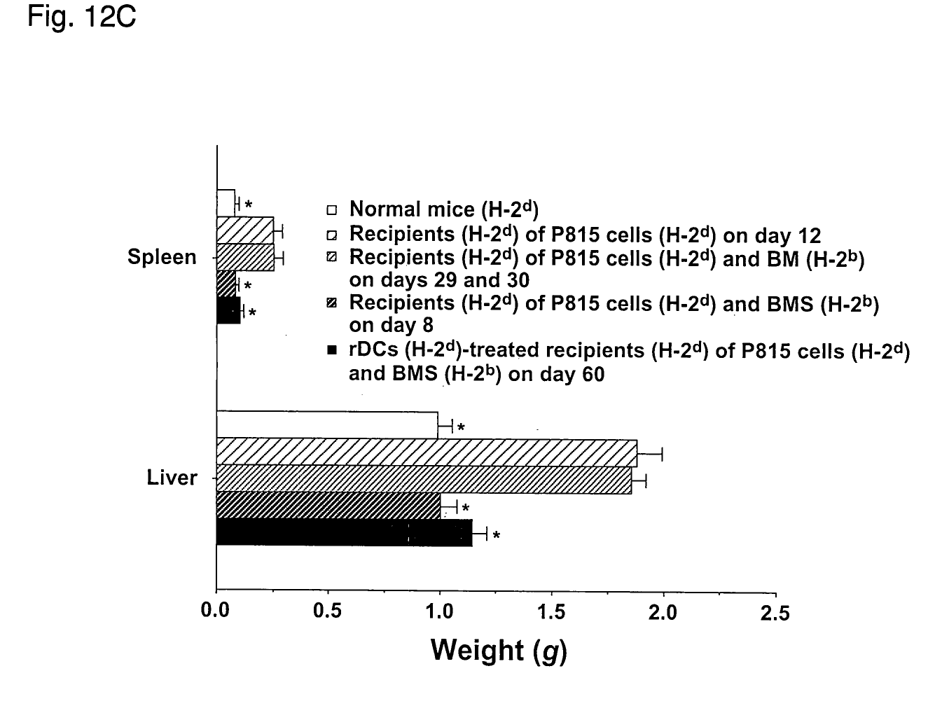

FIG. 12 shows the results of observation concerning the survival of mice (FIG. 12A), changes in body weights (FIG. 12B), and weights of livers or spleens (FIG. 12C). All mice that had been systemically irradiated with radiations died, their body weights decreased, and splenohepatomegaly was observed until the 12th day. In contrast, life prolongation was observed until the 30th day in the group to which only BMC had been administered, although death involving splenohepatomegaly, which was presumably caused by leukemia, was observed. While all mice died in the period up until the 8th day in the group to which BM and BMS had been administered, life prolongation of 60 days or longer and liver and spleen weight increases were observed in the group to which murine immunoregulatory DCs had been further administered. This indicates that anti-graft-versus-host disease effects were observed and graft-versus-leukemia effects could be maintained in the group to which murine immunoregulatory DCs had been administered.

### EXAMPLE 14

### Murine Immunoregulatory DCs Suppress Developed Type II Collagen-Induced Arthritis

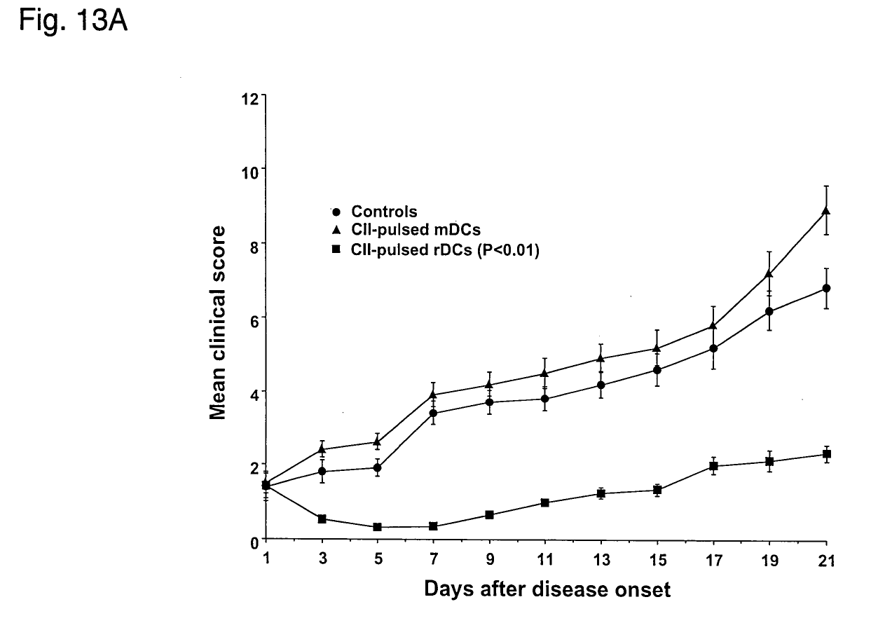

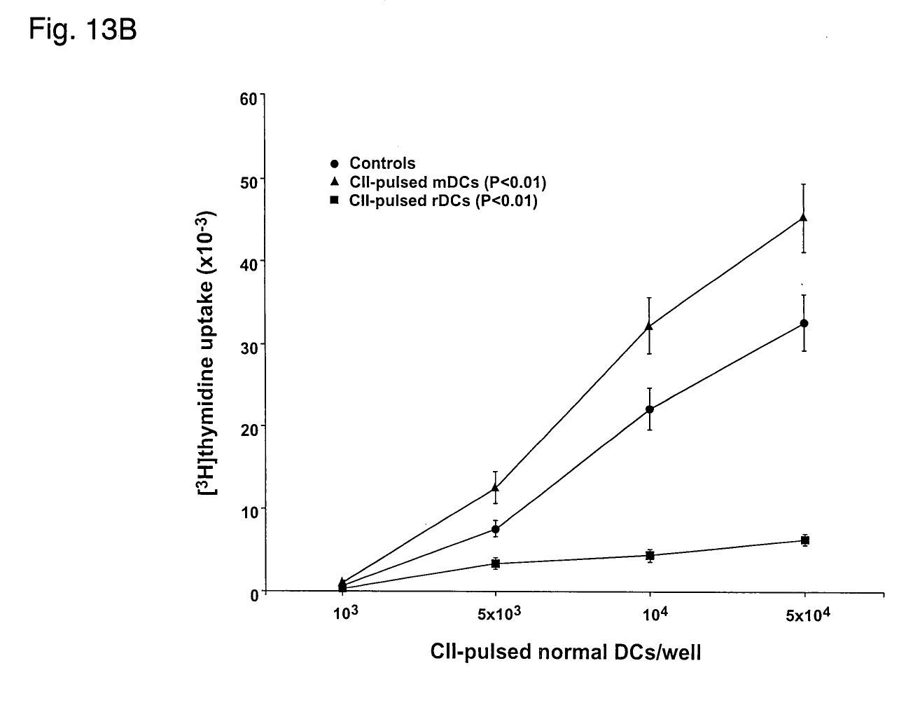

Activity of murine immunoregulatory DCs upon type II collagen-induced arthritis was examined. Arthritis was induced by subcutaneously administering 100 μg of bovine type II collagen (CII) to DBA/1 mice. CII, which had been used for sensitization, was administered as an emulsion together with Freund's complete adjuvant (Difco, Detroit, Mich.). Arthritis was observed every other day, and the results of observation were scored. Criteria were as follows: 0=no change; 1=slight erythema and edema; 2=advanced erythema and edema; and 3=deformity involving joint flexion. The maximal score would be 12 for total of 4 criteria. The day when arthritis had been developed was determined to be day 1, and murine normal mature DCs or murine immunoregulatory DCs were administered through caudal veins on that day. A group to which DCs had not been administered was provided as a control. Murine normal mature DCs were prepared in a manner as described in Example 6, these cells were cultured in the presence of CII (1 μg/ml) for 24 hours, and the culture products were then administered. As a result, murine immunoregulatory DCs more significantly suppressed the development of arthritis compared with the control group and the group to which murine normal mature DCs had been administered (FIG. 13A). Ten days after the development of arthritis, T cells derived from murine inguinal lymph nodes and subgenual lymph nodes were isolated, and their reactivity with murine normal mature DCs cocultured with CII was examined in the following manner. Lymphocytes were isolated from murine inguinal lymph nodes and subgenual lymph nodes using a Lympholyte-M (Cedarlane), and the isolated lymphocytes were subjected to negative selection using anti-Ly76, B220, Ly-6G, I-A/I-E, and a magnetic-beads-coupled anti-rat IgG antibody to prepare T cells. CII-pulsed DCs were prepared in the following manner. Murine iDCs were cultured in the presence of CII (1 μg/ml) for 24 hours, and the obtained DCs were further cultured in the presence of LPS (1 μg/ml) for 3 days. The obtained T cells (10⁵ cells) and X-ray (15 Gy)-irradiated murine normal mature DCs (10³ to 5×10⁴ cells) were cultured on a 96-well plate for 5 days, and cell growth assay was carried out. As a result, T cells derived from mice to which murine normal mature DCs had been administered exhibited significantly elevated reactivity compared with the control group, and the reactivity of T cells derived from mice to which murine immunoregulatory DCs had been administered was lowered (FIG. 13B).

### Industrial Applicability

As described in the Examples, immunoregulatory DCs stimulated with IL-10 and TGF-β induce antigen-specific anergy to T cells and suppress reactivation of activated T cells (Example 2). Also, the aforementioned immunoregulatory DCs suppress graft-versus-host disease after xenogeneic transplantation caused by T cells (e.g., Example 5). Further, immunoregulatory DCs suppress immune-related diseases (Example 14). As described in these Examples, the immunoregulatory DCs of the present invention suppress rejection caused along with cell, organ, or tissue transplantation, have therapeutic effects on graft-versus-host disease while maintaining graft-versus-leukemia effects, and also have therapeutic effects on autoimmune and allergic diseases.

All publications cited herein are incorporated herein in their entirety. A person skilled in the art would easily understand that various modifications and changes of the present invention are feasible within the technical idea and the scope of the invention as disclosed in the attached claims. The present invention is intended to include such modifications and changes.

1. A method of preparing human immunoregulatory dendritic cells by culturing human dendritic cells or their precursor cells in vitro with IL-10 and TGF-β.

2. The method of preparing human immunoregulatory dendritic cells according to claim 1, wherein the human dendritic cells are derived from human monocytes.

3. A method of preparing human immunoregulatory dendritic cells comprising culturing human monocytes in the presence of GM-CSF, IL-4, IL-10, and TGF-β.

4. The method of preparing human immunoregulatory dendritic cells according to claim 3, wherein culture is further conducted in the presence of at least one of TNF-α and LPS.

5. The method of preparing human immunoregulatory dendritic cells according to claim 1, wherein said human immunoregulatory dendritic cells are further cultured in the presence of an antigen existing in a tissue or organ associated with a disease to be treated.

6. The method of preparing human immunoregulatory dendritic cells according to claim 5, wherein the disease is an autoimmune or allergic disease.

7. Human immunoregulatory dendritic cells prepared by the method according to claim 1.

8. The human immunoregulatory dendritic cells according to claim 7, wherein expression levels of CD83, CD40, CD80, and CD86 are significantly lower than those in mature human dendritic cells that were not cultured in the presence of both IL-10 and TGF-β.

9. The human immunoregulatory dendritic cells of claim 7, which are capable of inducing antigen-specific anergy to allogeneic CD4⁺ T cells in vitro, suppressing reactivation of activated allogeneic CD4⁺ T cells and CD8⁺ T cells, inducing allogeneic and naive CD4⁺ T cells and CD8⁺ T cells to become CD4⁺ CD25⁺ immunoregulatory T cells and CD8⁺ CD28⁻ immunoregulatory T cells, respectively, and inducing immune-suppressing responses such as the suppression of graft-versus-host disease after xenogeneic transplantation in a human T cell-transplanted immunodeficient mouse through administration of the aforementioned cells to which xenoantigens have been imparted.

10. A pharmaceutical composition comprising the human immunoregulatory dendritic cells according to claim 7.

11. The pharmaceutical composition according to claim 10, which suppresses graft rejection caused along with cell, organ, or tissue transplantation.

12. The pharmaceutical composition according to claim 10, which can be used for treating graft-versus-host disease.

13. The pharmaceutical composition according to claim 10, which can be used for treating an autoimmune or allergic disease.
